(19) United States
(12) Patent Application Publication
(10) Pub. No.: US 2021/0116443 A1
(43) Pub. Date: Apr. 22, 2021

(54) Allosteric Split Trehalase Biosensor

(71) Applicant: Creative Protein Solutions Inc., Calgary (CA)

(72) Inventor: Jeroen De Buck, Calgary (CA)

(21) Appl. No.: 17/137,002

(22) Filed: Dec. 29, 2020

Related U.S. Application Data
(63) Continuation-in-part of application No. 16/289,357, filed on Feb. 28, 2019, now Pat. No. 10,900,960.
Application No. 16/289,357 is a continuation of application No. PCT/CA2017/051033, filed on Sep. 1, 2017.
(60) Provisional application No. 62/383,107, filed on Sep. 2, 2016.

(51) Int. Cl. G01N 33/535; C12Q 1/34; G01N 33/542; C12Q 1/00; C12Q 1/54

(52) U.S. Cl. CPC G01N 33/535; C12Q 1/34; G01N 33/542; G01N 2333/924; C12Q 1/54; C12Y 302/01028; C12Q 1/006

(57) ABSTRACT

The present disclosure relates to a method referred to herein as the “split trehalase assay biosensor” (also referred to herein as “STIGA”) is based on the use of engineered E. coli trehalase to detect analytes such as antibodies in a sample. The trehalase is engineered in a way such that the enzyme is split into two inactive fragments (N-terminal fragment H and C-terminal fragment A) with antigens fused to both fragments. When bivalent antibodies react specifically with the fused antigens, two inactive trehalase fragments are brought together in close proximity to restore the activity of trehalase. The restored trehalase will hydrolyze trehalose into two glucose molecules that can be measured using existing glucose detection methods such as glucometer, Benedict's reagent, or ACCU-CHEK AVIVA® glucose test strips.

### CROSS-REFERENCE TO RELATED APPLICATIONS

This application is a continuation-in-part application of U.S. application Ser. No. 16/289,357 entitled “ALLOSTERIC SPLIT TREHALASE BIOSENSOR” filed on Feb. 28, 2019, which is a continuation of International Patent Application No. PCT/CA2017/051033, entitled “ALLOSTERIC SPLIT TREHALASE BIOSENSOR”, filed on Sep. 1, 2017, which claims priority to and the benefit of the filing of U.S. Provisional Patent Application No. 62/383,107, entitled “ALLOSTERIC SPLIT TREHALASE BIOSENSOR”, filed on Sep. 2, 2016, and the specification and claims thereof are incorporated herein by reference.

### SEQUENCE LISTING

The instant application contains a Sequence Listing which has been submitted electronically in ASCII format and is hereby incorporated by reference in its entirety. Said ASCII copy, created on Dec. 28, 2020, is named CPCSplittrehalaseassay28Dec2020_ST25.txt and is 253,413 bytes in size.

### TECHNICAL FIELD

The present disclosure generally relates to biosensors. More particularly, the present disclosure pertains to biosensors for detection of antibodies that are reactive with bacteria, viruses, fungi, parasites, irritants, and polypeptides, and to biosensors for detection of other biological analytes.

### BACKGROUND

Although mobile and wearable technology are omnipresent, the promise to sense and report a variety of meaningful health related signals to the user and play an integral role in the health care system has not been achieved. Many biosensor devices have been developed (optical, electrical, electrochemical, and mass-based). However, only a few have made it into clinical practice or into daily use. The main reasons for this are their poor performance in clinical samples (blood, urine, saliva, sputum), insufficient sensitivity or specificity, expensive supporting equipment, or high cost of production.

### BRIEF SUMMARY

The present disclosure generally relates to a split enzyme assay for detection of selected analytes in a sample. Disclosed herein are compositions, biosensors, kits and methods relating to splitting a selected enzyme into two fragments, separately binding the fragments to one or more complexing domain(s), and then contacting the complexed fragments with a biological sample that potentially contains a target analyte. If the target analyte is present in the biological sample, the analyte will bind to the two enzyme fragments thereby restoring the functionality of the split enzyme.

According to one aspect, the restored enzyme functionality can be detected with an electrode measuring electrostatic charges produced as a result of enzyme activity.

According to another aspect, the restored enzyme functionality can be detected by adding a suitable substrate, and then colorimetrically measuring substrate catabolism.

According to one embodiment, the present disclosure relates to compositions, biosensors, kits and methods relating to splitting of a trehalase enzyme for use in the present split enzyme assays, referred to herein as a “split trehalase assay biosensor” and alternatively as a “split TreA assay”, and based on the use of engineered Escherichia coli trehalase to detect analytes in a biological sample. The trehalase is engineered in a way such that the enzyme is split into two inactive fragments (N-terminal fragment H and C-terminal fragment A) with antigens fused to both fragments. When bivalent antibodies react specifically with the fused antigens, two inactive trehalase fragments are brought in close proximity to restore the activity of trehalase. Alternatively, any other mechanism that brings the trehalase fragments in close proximity under specific conditions will restore the activity of trehalase. The restored trehalase will hydrolyze trehalose into two glucose molecules which can be measured using existing glucose detection methods such as glucometers, test strips, enzyme assays, Benedict's reagent, and the like.

### BRIEF DESCRIPTION OF THE DRAWINGS

The present disclosure will be described in conjunction with reference to the following drawings.

FIG. 1 shows a schematic illustration of the split TreA assay disclosed herein applied to the detection of antibodies.

FIG. 2 is a schematic representation of the different embodiments of split TreA assays disclosed herein.

FIG. 3 is a schematic representation of the alternative frame folding of TreA domains (SEQ ID NOs: 76, 77, 78) applied to detect antibodies and bacterial cells.

FIG. 4 is a schematic representation of a split TreA monovalent antibody sensor (SEQ ID NOs: 25, 26, 28, 29) according to one embodiment of the present disclosure.

FIG. 5 is a schematic representation of an exemplary fusion of a glucose oxidase molecule with TreA^{N} (SEQ ID NOs: 28, 79).

FIG. 6 is a schematic representation of two approaches for conversion of antibodies into signals detectable with an exemplary trehalase biosensor according to an embodiment of the present disclosure.

FIG. 7 is a micrograph showing glucose production, using Benedict's reagent, by TreA^{C} and TreA^{N} fragments that were purified separately and incubated with the substrate/trehalose separately and together (blue: no detectable glucose levels; orange: detectable glucose levels) (SEQ ID NOs: 20, 21, 22, 23).

FIG. 8 is a schematic representation of an example of a complementation of split TreA on Ni-NTA resin with the N-terminal fragment TreA^{N} (red), C-terminal TreA^{C} (blue), HIS tag (green) and the Ni-NTA resin (violet). Recombinant TreA^{C} and TreA^{N} fragments, HIS-tagged at N or C terminus, were immobilised on Ni-NTA resin, either separately or together (n=3) (SEQ ID NOs: 20, 21, 22, 23).

FIG. 9A is a chart showing trehalase activity and the resulting glucose concentration in complementations prepared as shown in FIG. 8 measured by GOx-HRP assay after 30 min, FIG. 9B is a chart showing trehalase activity and the resulting glucose concentration measured by ACCU-CHEK AVIVA® glucose strips after 30 min, and FIG. 9C is a chart showing trehalase activity of HIS-tagged TreA and TreA^{N} fragments were immobilised on Ni-NTA resin, either separately or together at pH 6 or at pH 7 after 5 min, using the GOx-HRP assay (SEQ ID NOs: 20, 21, 22, 23).

FIG. 10 is a schematic representation of an example of a complementation of split TreA induced by antibodies with the N-terminal fragment TreA^{N} (red), C-terminal TreA^{C} (blue), peptide antigens (green), and cognate antibodies (violet) (SEQ ID NOs: 20, 21, 22, 23, 24, 25, 26, 27).

FIG. 11A is a chart showing trehalase activity after incubation of HA-tagged TreA and TreA^{N} fragments (SEQ ID NOs: 24, 25, 26, 27) with or without anti-HA monoclonal antibodies using the GOx-HRP assay in complementations prepared as shown in FIG. 10, and FIG. 11B is a chart showing Trehalase activity after incubation of HIS-tagged TreA^{C} and TreA^{N} fragments (SEQ ID NOs: 20, 21, 22, 23) with or without anti-HIS monoclonal antibodies using the Anti-HIS assay.

FIG. 12A is a chart showing trehalase activity after incubation of TreA and TreA^{N} fragments fused to HIV capsid protein p24 (HIV) (SEQ ID NOs: 30, 31) with or without anti-HIV (p24) rabbit antiserum using the Anti-HIV assay in complementations prepared as shown in FIG. 10, and FIG. 12B is a chart comparing the measurement of glucose detection using the GOx-HRP assay, a glucometer and Benedict's reagent (SEQ ID NO 24, 25, 26, 27).

FIG. 13A is a chart showing the sensitivity of the TreA split Ab detection assay demonstrated by incubating TreA^{C} fragment and TreA^{N} fragment carrying HA tags (SEQ ID NOs: 24, 26) with decreasing amounts of Anti-HA tag mAb or without. (n=3)(glucose levels were measured by the GOx-HRP assay every 5 minutes for 1 h), FIG. 13B is a chart showing the time response of TreA spilt detection assay with and without anti-HA (n=3), and FIG. 13C is a chart showing the specificity of the TreA split Ab detection assay demonstrated by incubating HIS-tagged TreA^{N} and TreA^{C} fragments (SEQ ID NOs: 21, 23) with anti-HIS tag mAb, anti-HA mAb or without antibodies (n=3)(glucose concentration was measured by GOx-HRP assay performed for 30 min).

FIG. 14 is a chart showing the sensitivity of a 3-enzyme assay (3EA) for detection of a series of concentrations of human IgGs quantified by complementation of split TreA fragments fused to protein G (SEQ ID NOs: 28, 29) wherein glucose oxidase, horse radish peroxidase, and O-dianisidine convert the de novo produced glucose by the complemented TreA into a colorimetric signal detectable at 450 nm OD.

FIG. 15 is a schematic representation of an example of complementations of split TreA induced throughout interaction with different antibody regions (variable (antigen binding) and constant region) with the N-terminal fragment TreA^{N} (red), C-terminal TreA^{C} (blue), antigen (green), protein G (pG) (green) and analyte (violet) (SEQ ID NOs: 25, 27, 28, 29).

FIG. 16A is a chart showing trehalase activity after incubation of TreA^{N} and TreA^{C} fragments either both fused to HA-tag or one fused to Protein G and the other to HA-tag in the presence of cognate monoclonal antibodies (n=3) in complementations prepared as shown in FIG. 15 (SEQ ID NOs: 25, 27, 28, 29), and FIG. 16B is a chart showing trehalase activity after incubation of TreA^{N} and TreA fragments both fused to Protein G in the presence bovine IgGs (SEQ ID NOs: 28, 29).

FIG. 17A is a schematic illustration showing the affinity of protein G (pG), protein A (pA) and protein L (pL) for regions within immunoglobulins, and FIG. 17B is a schematic illustration of TreA fragment fusions with pG, pA, and pL (SEQ ID NOs: 28, 29, 32, 33, 34, 35).

FIG. 18A is a chart showing complementation of split TreA fusions by IgG over time, and FIG. 18B is a chart showing detection of human IgG, IgM or IgA (40 nM) with TreA fragments (TreAN and TreAC) fused to protein G (pG), protein A (pA), or protein L (pL). The 3EA assay was used to detect de novo glucose production by the complemented TreA (SEQ ID NOs: 28, 29, 32, 33, 34, 35).

FIG. 19A is a schematic representation of an example of a split TreA complementation occurring on a pathogen's surface with the N-terminal fragment TreA^{N} (red), C-terminal TreA^{C} (blue), sensors (green), analyte (violet), and FIG. 19B is a chart showing trehalase activity after incubation of TreA^{C} and TreA^{N} fragments fused with S. aureus specific binding peptides (SAbp) in the presence of S. aureus, S. epidermidis or in the absence of bacteria (n=3) is a schematic representation of the STIGA detection mechanism used to estimate/determine the quantity of IgGs in samples (SEQ ID NOs: 40, 41, 42, 43).

FIG. 20 is a chart showing detection of Mycobacterium avium subsp. paratuberculosis (MAP) whole bacterial cells with an exemplary split TreA detection assay with incorporated MAP specific binding peptide (Mp3) (SEQ ID NOs: 44, 45, 46, 47).

FIG. 21 is a chart showing detection of Listeria monocytogenes whole bacterial cells with an exemplary split TreA detection assay with cognate incorporated variable fragment (scFv) specific for L. monocytogenes ActA (SEQ ID NOs: 49, 50.

FIG. 22 is a chart showing detection of small analytes ATP by monitoring trehalase activity when TreA C fragment and TreA N fragment with C terminal fusions with ATPase subunit F0F1 ε are incubated with 10 mM of ATP or GTP in the presence of 0.25 M trehalose (SEQ ID NOs: 51, 52).

FIG. 23 is a schematic representation of an example of a split TreA complementation split TreA complementation occurring upon protein-protein interaction and protein aggregation with the N-terminal fragment TreA^{N} (red), C-terminal TreA^{C} (blue), leucine zippers or recombinant PrP (green) (SEQ ID NOs: 53, 54, 55, 56, 58, 59).

FIG. 24A is a chart showing trehalase activity after co-incubation of TreA and TreA^{N} fragments fused to complementary leucine zippers (Ei; Ki) (n=3) (SEQ ID NOs: 53, 54, 55, 56), and FIG. 24B is a chart showing trehalase activity after aggregation of TreA and TreA^{N} fragments fused to PrP induced by co-dialysis (n=3) (SEQ ID NOs: 58, 59, 20, 22).

FIG. 25A is a chart showing trehalase activity after incubation of lyophilized TreA^{N} and TreA^{C} fragments fused to complementary leucine zippers (Ki+Ei), resuspended in acidified bovine blood or milk (pH 6) (n=3) (glucose concentration was measured with ACCU-AVIVA® glucose strips after 3 h of incubation, residual glucose detected in both blood and milk was subtracted from measured signals), and FIG. 25B is a chart showing complementation of lyophilized TreA^{N} and TreA^{C} carrying complementary leucine zippers (Ki+Ei) fragments compared to fresh preparation (n=3). Glucose concentration was measured with ACCU-AVIVA® glucose strips after 1 h of incubation) (SEQ ID NOs: 55, 56).

FIG. 26 is a chart showing trehalase activity after incubation of lyophilized TreA^{N} and TreA^{C} fragments fused to either protein G or complementary leucine zippers (Ki+Ei), resuspended in pure saliva (glucose concentration was measured with ACCU-AVIVA® glucose strips after 2 h) (SEQ ID NOs: 55, 56).

FIG. 27A is a chart showing trehalase activity after complementation of fusions of split trehalase and split inteins fragments and excision of the intein and ligation of the trehalase fragments, and FIG. 27B is a SDS-PAGE gel showing intein and ligation of the trehalase fragments of a biodetector according to an embodiment of the present disclosure (SEQ ID NOs: 17, 61, 62).

FIG. 28 is a schematic representation of the STIGA detection mechanism used to estimate/determine the quantity of IgGs in samples (SEQ ID NOs: 28, 29).

FIG. 29 is a schematic representation of the methods used for the STIGA and the STIGA^{GLU} assays (SEQ ID NOs: 28, 29).

FIG. 30A is a scatter plot comparing IgG concentration measured by STIGA (OD 450) and by RID (ml/mg) in dairy colostrum (n=60) and FIG. 30B is a scatter plot comparing IgG concentration measured by STIGA (OD 450) and by RID (ml/mg) in beef colostrum (n=64) (SEQ ID NOs: 28, 29).

FIG. 31A is a scatter plot comparing IgG concentration determined by RID (mg/ml) and by STIGA (OD 450) in dairy calf sera (n=83), and FIG. 31B a scatter plot comparing IgG concentration determined by RID (mg/ml) and by STIGA (OD 450) in beef calf sera (n=84) (SEQ ID NOs: 28, 29).

FIG. 32A is a scatter plot of IgG concentration measured by TreA IgG assay (mM of glucose measured with a glucometer) and concentration determined by RID (mg/ml) for dairy colostrum samples (n=14), and FIG. 32B is a scatter plot of IgG concentration measured by TreA IgG assay and by RID for beef colostrum samples (n=14) (SEQ ID NOs: 28, 29).

FIG. 33A is a scatter plot of IgG concentration measured by TreA IgG assay and by RID for dairy calf serum samples (n=22), and FIG. 33B is a scatter plot of IgG concentration measured by TreA IgG assay and by RID for beef calf serum samples (n=22) (SEQ ID NOs: 28, 29).

FIG. 34A is a schematic representation of complementation of split TreA fragments fused to heterodimerizing peptides (EI and KI) whereby one fragment is also fused to glucose dehydrogenase (GDH) and generation of a colorimetric signal by GDH wherein different combinations of TreA fragments and peptides are combined to demonstrate that the complementation occurs only when complementary heterodimerizing peptides are fused to the correct fragments, and FIG. 34B shows a corresponding colorimetric assay (570 nm OD) using 1-methoxy-5-methylphenazinium methyl sulfate (mPMS) as the mediator, thiazolyl blue tetrazolium bromide (MTT) as the substrate, and pyrroloquinoline quinone (PQQ) as co-factor, to convert the de novo glucose produced by the complemented TreA into the colorimetric signals (SEQ ID NOs: 53, 54, 55, 71, 72).

FIG. 35 is a schematic representation of a split TreA calcium sensor according to another embodiment of the present disclosure in Example 5.

FIG. 36 is a chart illustrating the detection of calcium ions (Ca²⁺) by the split TreA calcium sensor shown in FIG. 35.

FIG. 37A-E are charts showing glucose production by the split TreA calcium sensor shown in FIG. 35, in solutions having (i) 25 mM of EDTA at pH 6 with 25 (▪), 10 (♦), 2.5 (▴), 1 (●) mM of Ca²⁺ ions (FIG. 37A), (ii) 10 mM of EDTA at pH 6 with 25 (▪), 10 (♦), 2.5 (▴), 1 (●) mM of Ca²⁺ ions (FIG. 37B), (iii) 2.5 mM of EDTA at pH 6 with 25 (▪), 10 (♦), 2.5 (▴), 1 (●) mM of Ca²⁺ ions (FIG. 37C), and (iv) 1 mM of EDTA at pH 6 with 25 (▪), 10 (♦), 2.5 (▴), 1 (●) mM of Ca²⁺ ions (FIG. 37D), while FIG. 37E is a chart showing Pearson correlation plots of glucose production by the split TreA calcium sensor with Ca²⁺ ions 25 (▪), 10 (♦), 2.5 (▴), 1 (●) mM of EDTA.

FIG. 38 is a chart illustrating the detection of bovine lactoferrin by a split TreA calmodulin sensor according to another embodiment of the present disclosure in Example 6.

FIG. 39A-B are schematic representations of the use of lactoferrin-sensitive reagents (FIG. 39A) and lactoferrin-insensitive reagents (FIG. 39B) with the split TreA calmodulin sensor disclosed in Example 6.

FIG. 40 is a chart illustrating glucose production by the split TreA calmodulin sensor disclosed in Example 6, with lactoferrin-sensitive reagents and lactoferrin-insensitive reagents in the presence of 4 mM CaCL₂ and 10 mM EDTA with increasing concentrations of bovine lactoferrin (“0” lactoferrin was set as 100% activity).

### DETAILED DESCRIPTION

The embodiments of the present disclosure generally relate to protein/enzyme fragment complementation assays that are routinely used to identify protein-protein interactions. In complementation assay, protein/enzyme is split into two fragments and reconstituted non-covalently to restore activity by other interacting proteins that are fused to fragments. Some examples of split proteins are luciferase, beta-lactamase, Gal4, beta-galactosidase, GFP and ubiquitin.

The efficacy of split trehalase assay biosensor disclosed herein is based on the following parameters: 1) split trehalase can restore its activity when its two fragments are brought together in close proximity; 2) either fragment alone does not possess enzymatic activity; 3) two fragments co-incubated together do not possess enzymatic activity without the specific complexing analyte; 4) only antibodies specific to fused antigens or other specific analytes interacting with the cognate fused (poly)peptides are capable of restoring enzymatic activity of trehalase; 5) there is no or very low level of endogenous trehalase activity in the biological sample to be tested.

Some aspects of the present disclosure pertain to splitting trehalase into N-terminal and C-terminal fragments and to fuse antigens, e.g. His-tag, HA-tag, or antigenic protein P24 to the end(s) of each fragment. The aspects of the present disclosure include the scheme of incorporating the antigens into the trehalase fragments. It investigates the position effects of the antigens with regard to the N-terminal or C-terminal of the fragments on the restored enzymatic activity.

Some aspects of the present disclosure pertain to expressing recombinant trehalase fragments in the E. coli strain BL-21 (DE3) knock-out for endogenous TreA gene (BI-21 ΔTreA) and purifying recombinant fragments using Ni-NTA columns. The assay is performed in a test tube by combining N-terminal and C-terminal fragments in a solution containing trehalose. A method to eliminate glucose present in the biological sample before the enzyme is activated is also within the scope of the present disclosure.

Another aspect of the present disclosure pertains to fusing one of the antigen-trehalase fragments (that is, N-terminal) to glucose oxidase or glucose dehydrogenase. This design allows the glucose generated by restored trehalase to be quickly converted to a signal that can be detected photospectrometrically or electrochemically.

For the successful creation of a split-reporter protein, several criteria must be met. Each protein fragment by itself should not exhibit any activity, the affinity of the fragments in the absence of attached interacting proteins should be negligible, and ideally the reassembled split-protein must provide an easily measurable read out.

The present disclosure pertains to split biosensor-linked immunodetector biosensors that are solution-based, homogenous, “mix and read” that rely on recognition of antibodies and other analytes coupled with enzymatic gain.

The biosensors disclosed herein include, but are not limited to, enzymes that can be split into two independent folding domains, which by themselves have little or no detectable activity, nor sufficient affinity for their complementary partner to lead to automatic complementation. If the latter is not naturally the case, it could still be accomplished by re-engineering residues at the interacting surfaces of the split fragments. This ensures a good signal-to-noise ratio. However, once brought together by the affinity of the detector for its analyte, activity is restored. Because of the enzymatic turn-over of a measurable product, this assay provides gain (amplification) to the signal associated with the presence of the targeted analyte.

This disclosure outlines many advantages to bio-detection: sensitivity, specificity, simplicity, use of simple (existing) readers, homogenous, compatibility with biological samples types and matrices, “mix and read” format. The solution-based, dual-recognition, split-enzyme linked detector systems will have broad applications where highly specific, sensitive, inexpensive, and portable detection of specific biological agents or substances are required. For example, for detecting analytes in a ‘field’ setting.

The biosensors disclosed herein may detect the presence of antibodies that are reactive with bacteria, viruses, fungi, irritants, and proteins.

The biosensors disclosed herein may also detect other target entities found in biological samples, for example, a cell, protein, peptide, hormone, cytokine, chemokine, nucleic acid, a virus, a bacterium, an organic molecule, a lipid, a fatty acid, a carbohydrate, a drug, an element, a toxin, a chemical, a metabolite, or a complex comprising two or more of any of the aforementioned items.

The target entities may be present in biological samples collected from animals or human individuals if the individuals are positive for the conditions being tested. Such target entities may be, for example markers of the condition, or they may be the actual toxin, drug, or pathogen being sought. Accordingly, the target entities (or components of the target entity) may be, for example, a nucleic acid, a ribonucleic acid, a polypeptide, a carbohydrate, a protein, a peptide, an amino acid, a hormone, a steroid, a vitamin, an ion, a metabolite, a chemical, an element, a derivative, an analogue, a polysaccharide, a lipid, a fatty acid, a lipopolysaccharide, a glycoprotein, a lipoprotein, a nucleoprotein, an oligonucleotide, an antibody, an immunoglobulin, a coagulation factor, a peptide hormone, a protein hormone, a non-peptide hormone, an interleukin, an interferon, a cytokine, a chemokine, a cell, a cell-surface molecule, a microorganism, a small organic molecule, a virion, a bacterium, a toxin, a drug, a cell membrane, a membrane fraction, a protein complex, an antigen, a hapten, a receptor, a macromolecule, or a molecular complex comprising two or more of any of the aforementioned items.

Many biosensor devices have been developed, for example, optical, electrical, electrochemical, and mass-based. However, only a few have made it into clinical practice or into home use. The main reasons for this are their poor performance in clinical samples (blood, urine, saliva, sputum), insufficient sensitivity or specificity, expensive supporting equipment, or high cost of production. Regardless, the markets are ready to adopt “Anything-Anywhere-Anytime-Anyone” biosensors if such biosensors meet certain expectations.

In order to fulfill the ‘Anyone’ requirement, a candidate biosensor should be extremely user-friendly (even for unskilled users), require non-invasive samples (for example, a pin-prick of blood, tears, or saliva) and minimal sample handling. Moreover, the biosensor should be quantitative and easily interface with mobile technology and thereby be compatible with emerging telehealth-based health care. To satisfy the ‘Anywhere’ requirement, a candidate biosensor must not require expensive equipment, must require no or minimal sample processing, and must be robust and portable. To satisfy the ‘Anytime’ requirement, a candidate biosensor should be accessible, cheap, and fast, and devoid of a need for a transport chain. To satisfy the ‘Anything’ requirement, a candidate biosensor should be sensitive, specific, and versatile. However, most biosensors currently available are optimized for a specific analyte or group of analytes, which restricts their applicability and broad adoption.

The most successful class of biosensor currently available on the market that meets the first three ‘A’ requirements, is glucometers used to monitor blood glucose concentrations. Currently available glucometers comprise a glucose-specific oxido-reductase (glucose oxidase or dehydrogenase) and an electrochemical transducer that converts enzyme activity into an electrochemical signal. A technology that makes use of this existing biosensor but adds versatility (i.e., ‘Anything’) by increasing the type of analytes that can be analysed, would have important advantages. The split Trehalase biosensors disclosed herein satisfy the ‘A’ requirements.

For successful creation of a split-reporter protein, several criteria must be met. Each fragment by itself should not exhibit activity, the affinity of the fragments in the absence of fused interacting proteins should be negligible, and the re-assembled split-protein must provide an easily measurable output. Despite its deceptive simplicity, identification of potential split proteins and their appropriate dissection sites is limited. Currently, there are a finite number of reported split proteins available for consideration, i.e., luciferase, fluorescent proteins, beta-lactamase, proteases, and the like.

Herein is disclosed a versatile detection platform based on the protein complementation principle that is able to detect a wide range of types of analytes. At the core of this platform is a glycolytic enzyme trehalase (TreA) localized in the periplasmic space of E. coli that catalyzes hydrolysis of trehalose into two glucose molecules. Production of glucose can be easily detected by a glucometer whereas the output signals from other split enzymes are less compatible with existing detectors and with detection in biofluids of humans and animals, e.g. fluorescence or luminescence from GFP or luciferase. Glucose detection has a quick and easy read-out with proven compatibility with complex samples without additional handling or processing.

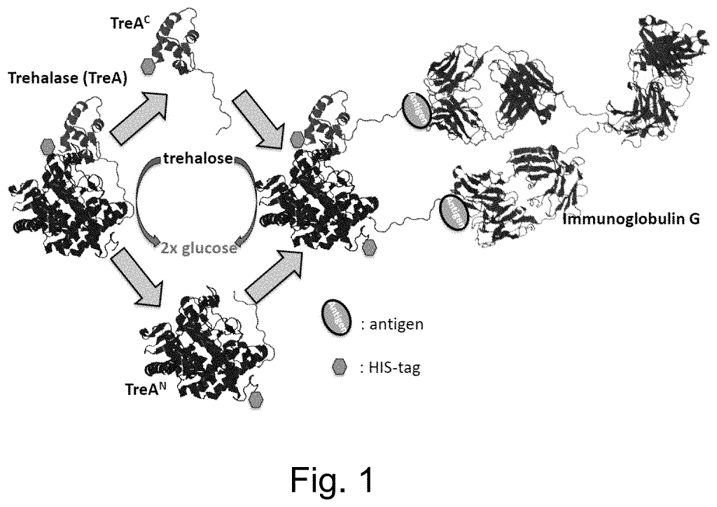

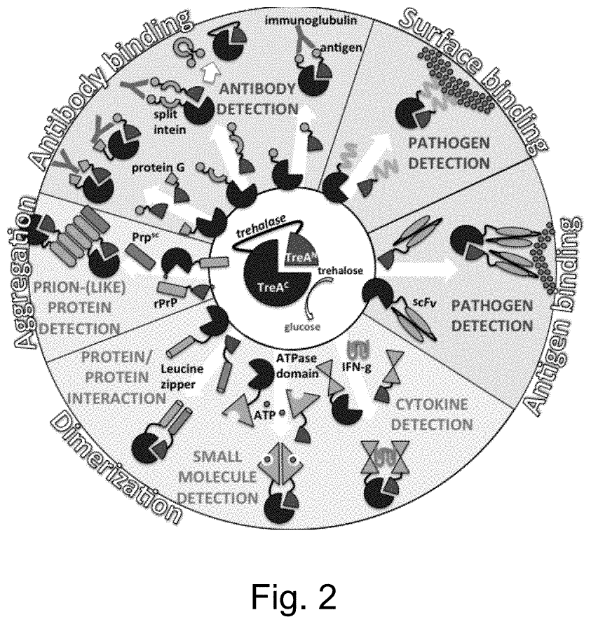

TreA has a bimodular structure with a connecting flexible linker, not unlike luciferase. Consequently, TreA, split at the site of this linker into two non-functional fragments, can be fused to sensor domains specific for an analyte of interest. The interaction/bond between analyte and sensor triggers complementation of two TreA fragments, which leads to activation of the enzyme. Suitable biosensors for detection of a wide variety of analytes, including antibodies, bacteria, viruses, small molecules, hormones, cytokines and prion-(like) protein based on various mechanisms to mediate complementation of reporter fragments, such as bivalent binding of antibodies, protein dimerization, protein aggregation, surface binding by using peptide aptamers, single chain fragment variables (scFv), antigens, receptor proteins and recombinant prion-like proteins as fusion partners to the reporter enzyme, are shown in FIGS. 1 and 2. Also, shown are complementation of TreA fragments by heterodimerizing coiled-coil peptides (˜leucine zipper) and the reconstitution of TreA by split intein fragments fused to split TreA fragments.

The present disclosure relates to a method for detection of analytes in biological samples with an engineered enzyme, for example the periplasmic TreA of E. coli that is split into two domains and thereby rendered inactive, and is provided with sensor polypeptides fused to both fragments. These polypeptides interact in the presence of the analyte and thereby bring the TreA fragments into close enough proximity such that protein complementation is induced. Accordingly, the present disclosure pertains to a replacement of a naturally occurring linker connecting the two domains with a conditional linker, whereby the conditional linker only materializes through when an analyte is present. The conditional linker comprises non-covalent or covalent bonds. In the case that the conditional linker comprises covalent bonds, split inteins may be used as the conditional linkers.

According to a first embodiment of the present disclosure, different interaction mechanisms enable this ‘complementation by a proximity’ mechanism, thereby enabling detection of a variety of analytes using different fusion partners (i.e., sensor (poly)peptides). Firstly, antibodies are bivalent and when each of both binding sites binds with a corresponding antigen fused to the TreA fragments, the fragments are kept in close enough proximity to restore the activity of the TreA. The restoration of TreA activity leads to hydrolysis of trehalose into two glucose molecules, an activity that is not present in either TreA fragment alone or in combined TreA fragments without the presence of a complexing antibody (FIG. 1).

According to a second embodiment of the present disclosure, peptide aptamers enable binding of TreA fragments onto a surface, for example a bacterial surface or the surface of a Ni-NTA resin bead, in close enough proximity for protein complementation to occur. Other examples of this mechanism include for example, peptides that specifically bind to bacterial cell envelopes, cell membranes, cell walls, viral particles, parasite surfaces, parasite cuticles, and the like.

According to a third embodiment of the present disclosure, single-chain fragment variables (i.e., antibody derivatives) with specificity for a bacterial surface protein, may be fused to bind TreA fragments close enough together to initiate protein complementation.

According to a fourth embodiment of the present disclosure, proteins that dimerize in the presence of specific small molecules such as ATP and estradiol, may be used to detect the presence. Similarly, protein G or protein A may be fused to the TreA fragments to thereby bind both heavy chains of IgG molecules, and can thereby complement the TreA activity. Additionally, heterodimerizing peptides fused to TreA fragments may complement enzymatic activity as would other pairs of (poly)peptides that have affinities for each other.

According to a fifth embodiment of the present disclosure, TreA fragments may be fused to recombinant proteins that have tendencies to aggregate. Co-aggregation of such fusion proteins may facilitate bringing the TreA fragments lose enough together to restore the TreA enzyme activity. Recombinant prion proteins may be incorporated to detect infectious prion proteins by the induction of misfolding and fibril formation in the recombinant prion proteins.

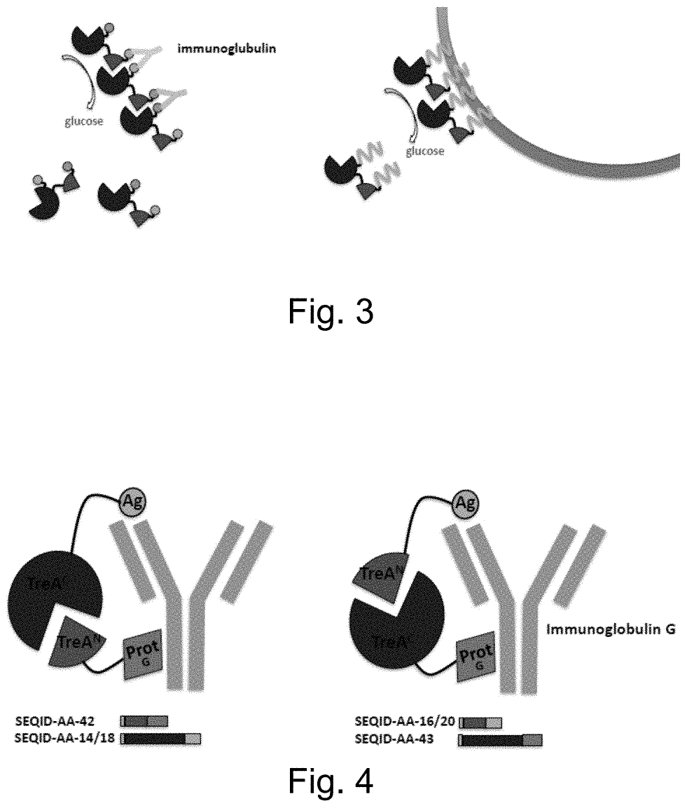

It is known that TreA may be engineered so that the N-terminal fragment is relocated and fused to the end of the protein (FIG. 3; also known as alternative frame folding (AFF)) with a linker that does not allow the correct bimodular conformation of the enzyme to occur thereby ensuring that this protein is not functional on its own (e.g., SEQ ID NOs: 76, 77). Two different antigens are fused to the N-terminus and C-terminus of this protein. These fusion proteins are produced in pairs whereby the same antigen is fused to the opposite terminus of the protein resulting in the ability of antibodies for either antigen to link the proteins together thereby complementing a N-fragment and a C-fragment of TreA, and thereby activating glycoside hydrolase activity. If both antigen-specific antibodies are present, the formation of fibrils are possible with activation of TreA activity. Fusing the same antigen to both ends of one protein is avoided because otherwise, a portion of binding antibodies will bind with both binding sites on a single fusion protein without restoring protein function. This AFF strategy can also be followed to construct a single bacterial surface-recognizing protein (SEQ ID NO: 78), for example, for Staphylococcus aureus by including peptide SA5-1 (SEQ ID NO: 38; FIG. 3).

TreA may be modified and engineered as outlined in some of the embodiments disclosed herein. The fragments of TreA may be produced recombinantly and purified. The signal peptide may be eliminated from the N-terminal fragment, and the naturally existing linker between both domains may be duplicated and kept in both fragments at their original positions. His-tags may be fused to all termini, sometimes with the inclusion of a small number of amino acids. Restriction enzymes sites may be added to the termini to allow cloning and subsequent insertion or deletion of peptides.

According to one aspect of the present disclosure, the compositions, biosensors, kits, and methods disclosed herein may be modified for use in the detection the presence of antibodies and other analytes in a solution or in a biological sample for example such as a fluid from animal or human exemplified by blood, serum, milk, sweat, semen, ejaculate, mucus, tears, saliva, plasma, secretions of the genitourinary tract, lymph fluid, urine, white blood cells, sputum, pleural fluid, ascites, sputum, pus, excrement, peritoneal fluid, cerebrospinal fluid, pleural fluid, pericardial fluid, amniotic fluid, synovial fluid, interstitial fluid, or any combination of such fluids, or any derivations of such fluids. Such biological samples are also referred to herein as “biofluids”.

The compositions, biosensors, kits, and methods disclosed herein may utilize a split-enzyme biosensor in which same (poly)peptide is fused to both fragments of the split enzyme. This antigen may a peptide or a polypeptide or a protein fragment or a protein domain or an entire protein. The antigen may be fused by incorporating the coding DNA sequence in the gene for the split enzyme fragment that is in turn, produced recombinantly in a cell expression system, for example a yeast cell expression system, a plant cell expression system, a fungal cell expression system, a mammalian cell expression system, or a bacterial cell expression system.

According to one embodiment of the present disclosure, both fragments of the enzyme portion of the sensors may be combined to form a biologically active enzyme when the fused antigens bind to a bivalent-complementing immunoglobulin. Such bivalent-complementing immunoglobulin may be of any isotype for example IgA, IgG, IgM, and the like. According to some aspects, the interaction of fusion partners with a selected analyte may also result in complementation of the fragments.

When TreA enzymes are used, the production of a reducing sugar (glucose) from a non-reducing sugar (trehalose) may be detected. Also, the resulting product, glucose, may be detected by known methods that use glucose-oxidase functionalized glucometers or alternatively, with glucose-dehydrogenase functionalized glucometers.

The embodiments of the present disclosure are based on the selection of an enzyme that can create a signal that is measurable in biological samples using known measuring methods and devices for example, glucometers. In essence, the methods disclosed herein convert the presence of the analyte into a signal, for example glucose, for which quick, practical, mobile, and individual user methods and devices have been developed. According to one aspect, a 2-step method according to the present disclosure may be used with conventional glucometer strips to measure glucose produced by the split TreA (FIG. 6). According to another aspect, a 1-step method according to the present disclosure condenses the two enzymatic functions by co-immobilization of split TreA and glucose oxidase onto electrode-containing test strips (FIG. 6). According to another aspect, a 1-step method according to the present disclosure condenses the two enzymatic functions by fusion of split TreA and glucose oxidase onto electrode-containing test strips (FIG. 6).

This is in contrast with known methods whereby the output, that is a measurable signal, is less compatible with existing detectors and with its detection in fluid samples from humans and animals, for example fluorescence or luminescence from Green fluorescent Protein (GFP) or luciferase, because these signals are quenched by components naturally present in these biological samples.

Colorimetric substrates of some split reporter proteins (beta-lactamase/nitrocefin or Centa) and beta-galactosidase (x-gal) are also difficult to quantify in a POC device (i.e., a point-of-care device) and do not provide good sensitivity or dynamic range. Some split enzymes only function as a selection marker and therefore require a cellular application, such as Trp1 wherein cells grow on a medium lacking tryptophan (i.e., a positive selection), or grow in the presence of antibiotics (beta-lactamase) lactamase (i.e., a negative selection).

Glucose detection is a quick and easy to read-out in POC devices, and has proven compatibility with live complex samples such as blood, serum, milk, and the like, without the requirements for additional handling and processing.

The compositions, biosensors, kits, and methods disclosed herein designed to provide an individual with an opportunity to perform all the steps of the method without the assistance of a health care professional. Accordingly, the individual can collect the sample themselves, manipulate components to allow contact of the sample with a solution comprising the reagents for a detection assay and then use a POC device such as a glucometer to show the results in as a digitized value or a quantitation value. Alternatively, other methods or reagents such as Benedict's reagent, may be used to detect glucose. The results generated with the assays disclosed herein, may be digitized for wireless transmission to smart phones or other such smart devices, and additionally, could be transmitted to a subject's health care professional.

This disclosure pertains to an antibody detection system that does not require the use of secondary antibodies or antibody binding proteins such as Protein A, Protein G, Protein L, or any hybrid or fusion of these proteins, except for production of the monovalent variant disclosed herein which relies on the incorporation of antibody-binding proteins. Neither does the present antibody detection system require additional conjugated enzymes. This disclosure also pertains to antibody detection systems that do not require any rinse steps, thereby providing a significant advantage in Point-of-Care methods.

Additionally, the production of glucose can be detected by the growth of microorganisms on a selected carbon source. This may be done in minimal media (MM) with a selected defined carbon source. In the case of trehalose as the substrate, this would require the use of trehalase-negative cells. This may be achieved by naturally trehalase-deficient organisms or by use of organisms in which one of the one or more the multiple genes coding for trehalases have been knocked-out (KO).

Disclosed herein is a TreA KO strain created and used to express all of the TreA constructs disclosed herein, in order to minimize or eliminate any background activity by native TreA that may be present in E. coli.

As an additional feature of the present disclosure, the gain of the method may be enhanced by combination with an additional enzyme such as glucose oxidase or glucose dehydrogenase, to convert the initial enzyme into another analyte that can be detected in a variety of ways.

This disclosure also relates to the fusion of one of the TreA fragments functionalized with an antigen or a peptide or another molecule to glucose oxidase or glucose dehydrogenase. When this glucose oxidase fused to a TreA fragment is complemented with the other TreA fragments, antibodies, or any other complexing or immobilizing analytes, the hydrolysis activity of TreA will be restored whereby the resulting glucose molecule may be taken up by the glucose oxidase domain. This may result in a faster assay because only a very localized concentration of glucose needs to be formed for the glucose oxidase to generate a signal that may be captured photospectrometrically or electrochemically with a glucometer, or alternatively, by capturing the electrons. Immobilization of the glucose oxidase/trehalase complex onto an electrode may provide a composition for sensitive and quick detection of the analyte. Alternatively, the fusion between GOX or GDH may established by intein protein ligation technology or other suitable biochemical ways.

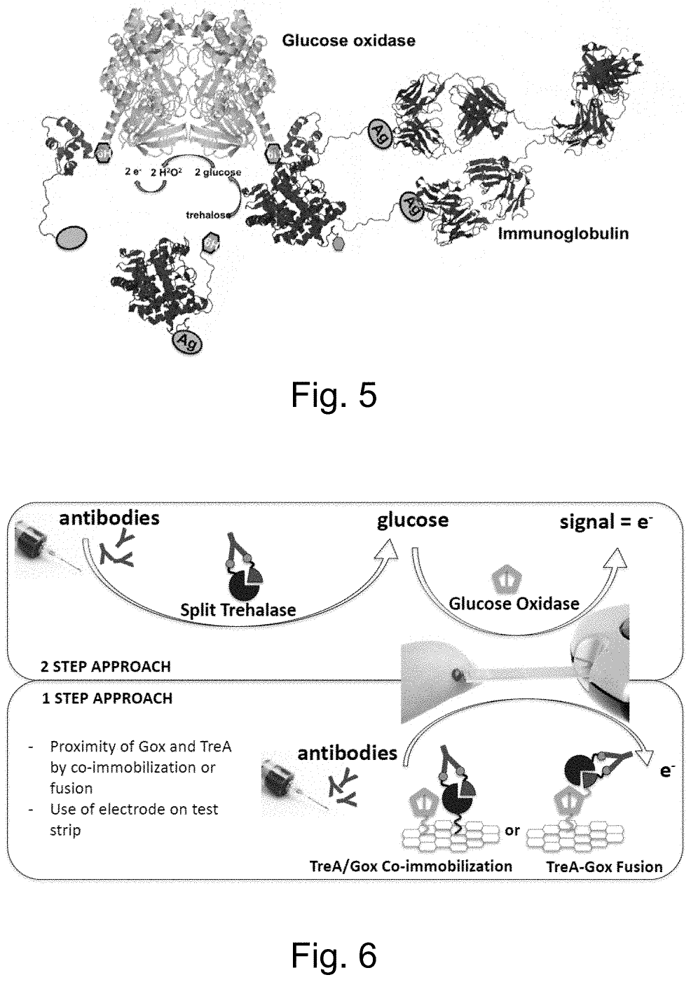

Important to note is the fact that glucose oxidase and glucose dehydrogenase typically form dimers, and therefore, a complex comprising two glucose oxidases, two trehalases and, for example, two IgG can be formed (FIG. 5).

The present disclosure also pertains to the discovery of the effect and impact of the locations of the antigens with regard to the N-terminal or C-terminal of the fragments. Antigens may be added in either of the four combinations whereby one combination leads to 2 N-terminal antigens, a second combination leads to 2 C-terminal antigens and the remaining two combinations each lead to a C- and N-terminal antigen. However, the binding of antigen specific antibodies or its capacity to restore the functionality of the TreA, is variable among the different combinations. For small antigenic peptides, it might be favorable for recognition by corresponding antibodies to be fused at the same terminus on both fragments in order to have identical presentations and conformations.

Also for other applications, (poly)peptides may be fused to either terminus of the fragments to achieve complementation of the fragments in interactions with a selected analyte (for example, binding to a bacterial surface). In some applications, the fusion order is determined by the structure of the fusion partner. For example, prion proteins can only be fused at the N-terminus.

Due to the fact that trehalase is an enzyme, signal amplification may be achieved with methods and compositions according to this disclosure, with more and more signal being produced over time with every specific antibody activating a trehalase protein. Periplasmic trehalase has a Vmax of 66 μmol/min/mg, pH optimum of 5-6. For trehalase, this amplification is intensified because every catalytic reaction generates not one, but two glucose molecules. Trehalases with higher activity from different sources could replace the periplasmic trehalase of E. coli.

The present disclosure also pertains to the linking of non-peptide molecules to the TreA fragments with covalent or non-covalent bonds. The covalent bonds may be achieved by protein chemistry as explained below. In some cases, this might require an insertion of specific amino acids into the sequence, especially at the termini, or it might require the fusion of a peptide that can be modified by protein chemistry to bind to the desired molecules. Examples of such molecules are lipids, carbohydrates, glycolipids, or combinations of these. These molecules might be antigens, epitopes, or enable dimerization in the presence of a specific analyte, or immobilize the fragments to a surface.

The present disclosure also pertains to compositions, biosensors, kits, and methods wherein one TreA fragment is fused to an immunoglobulin heavy chain binding molecule (for example, protein A or G or A/G or L) and the other fragment is fused to the antigen (FIG. 4). In this case, the bivalent nature of immunoglobulins is not utilized. However, complementation on two sides of the immunoglobulin is possible.

Previously several split proteins and enzymes have been produced. The specific properties of the split enzymes often limit their use to specific applications. Split TreA offers advantages to a variety of applications not yet found in the existing list of split proteins or not yet found in this combination.

TreA does not require cofactors, including metals, or specific cations or anions.

TreA has high activity at a neutral pH, the pH of most relevant biological samples for the detection of antibodies, although its optimum is around pH 5-6.

TreA is highly active at room temperature, that is in a range of about 20° C. to about 25° C.

TreA's product, glucose, can be measured directly in complex biological samples such as blood without any additional sample preparation, as demonstrated by the applications of the glucometers.

Split ° C. has the advantage of not resulting in self-complementation between the fragments without a linking molecule or bond. Self-complementation as seen in split beta-galactosidase (lacZ) largely eliminates the possibility of conditional complementation.

Furthermore, split ° C. fragments do not have any residual activity on their own or in combination without a specific complexing analyte. This is in contrast to split GFP or the beta-galactosidase complementation reaction whereby relatively low concentrations of both fragments will lead to fluorescence or enzymatic activity, respectively. This provides a method whereby the background signal is minimal or non-existent.

The existence as dimers of some split proteins or candidate split proteins can also be an important consideration or sometimes disadvantage (for example, PCNA).

A more practical advantage of using TreA is that the TreA molecular structure is relatively easy to fold and refold. Furthermore, it appears that the split fragments fold correctly independently of each other. In other proteins, refolding only happens at the complementation stage (for example, GFP, split inteins, DnaE), likely putting restrictions on the physicochemical properties of the reaction solution.

TreA has absolute substrate specificity for trehalose.

Positive signal: Split TreA provides one of the only antibody detection systems where a positive signal is created, in contrast to several other methods where a reduction of enzymatic activity indicates the presence of specific antibodies. In said previous methods, steric hindrance or inhibition by conformational changes by binding of antibodies to antigens fused to the enzyme are achieved leading to reductions in activity. Methods that are based on decreasing of activity are typically less sensitive.

It is known that TreA activity is non-existent or extremely low in most biological samples from vertebrates. Only two trehalose-hydrolyzing activities are present in vertebrates, with the exception of fecal matter that might have trehalose originating from fecal-associated microbiota. These activities in humans display precise locations, often acting as intrinsic glycoproteins of the microvillus intestinal mucosa and renal brush-border membranes linked by a GPI-anchor, as shown by their selective solubilization by phospholipase C. This dual enzymatic activity points to a strict control and rapid degradation of the trehalose ingested in the diet, preventing it from being accumulated even in transitory or low levels. Intestinal TreA is in all probability, the sole enzyme responsible for the hydrolysis of ingested trehalose (mushrooms and honey among other foods, are rich in the non-reducing disaccharide). Notably, intestinal TreA is never released into the bloodstream and is tightly attached to the external surface of the microvilli of enterocytes, resisting all proteolytic treatments. As a result, if the split TreA methods are applied in biological samples derived from animals, then all the measurable TreA activity results from the presence of the analyte and corresponding activation of the biosensor. This freedom or low level of TreA activity in biological samples is in contrast with the high activity of other potential reporter enzymes such as catalases of peroxidases.

Elimination of background glucose: Another advantage is that all free glucose present in biological samples can be quickly, efficiently, and completely eliminated before the de novo production of glucose is initiated. This elimination of glucose can be done by chemical or enzymatic methods, including the use of metaperiodate and glucose oxidase or glucose dehydrogenase. In other cases, de novo generated glucose will be distinguished from background glucose by measuring differential glucose concentrations or by measuring rate of increase of the glucose concentration.

Also of importance is the naturally low concentration of glucose in milk including breast milk (for example, about 0.331 mM). In some applications of the compositions and methods disclosed herein, it may be possible to disregard this initial low concentration glucose and measure de novo produced glucose by the specific antibodies or analytes being detected. In other applications, it may be necessary to eliminate the initial levels of glucose present in samples before use of the compositions and methods disclosed herein.

Unless otherwise defined, all technical and scientific terms used herein have the same meaning as commonly understood by one of ordinary skill in the art to which this invention belongs. In order that the invention herein described may be fully understood, the following terms and definitions are provided herein.

The word “comprise” or variations such as “comprises” or “comprising” will be understood to imply the inclusion of a stated integer or groups of integers but not the exclusion of any other integer or group of integers.

The term “nucleic acid” used herein refers to a polymeric compound comprised of covalently linked subunits called nucleotides. Nucleic acid includes polyribonucleic acid (RNA) and polydeoxyribonucleic acid (DNA), both of which may be single-stranded or double-stranded. DNA includes cDNA, genomic DNA, synthetic DNA, and semisynthetic DNA.

The term “gene” used herein refers to an assembly of nucleotides that encode a polypeptide, and includes cDNA and genomic DNA nucleic acids.

The term “recombinant DNA molecule” used herein refers to a DNA molecule that has undergone a molecular biological manipulation.

The term “vector” used herein refers to any means for the transfer of a nucleic acid into a host cell. A vector may be a replicon to which another DNA segment may be attached so as to bring about the replication of the attached segment. A “replicon” is any genetic element (e.g., plasmid, phage, cosmid, chromosome, virus) that functions as an autonomous unit of DNA replication in vivo, i.e., capable of replication under its own control. The term “vector” includes plasmids, liposomes, electrically charged lipids (cytofectins), DNA-protein complexes, and biopolymers. In addition to a nucleic acid, a vector may also contain one or more regulatory regions, and/or selectable markers useful in selecting, measuring, and monitoring nucleic acid transfer results (transfer to which tissues, duration of expression, etc.).

The term “cloning vector” used herein refers to a replicon, such as plasmid, phage or cosmid, to which another DNA segment may be attached so as to bring about the replication of the attached segment. Cloning vectors may be capable of replication in one cell type, and expression in another (“shuttle vector”).

A cell has been “transfected” by exogenous or heterologous DNA when such DNA has been introduced inside the cell. A cell has been “transformed” by exogenous or heterologous DNA when the transfected DNA effects a phenotypic change. The transforming DNA can be integrated (covalently linked) into chromosomal DNA making up the genome of the cell.

The term “nucleic acid molecule” used herein refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester anologs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms.

Modification of a genetic and/or chemical nature is understood to mean any mutation, substitution, deletion, addition and/or modification of one or more residues. Such derivatives may be generated for various purposes, such as in particular that of enhancing its production levels, that of increasing and/or modifying its activity, or that of conferring new pharmacokinetic and/or biological properties on it. Among the derivatives resulting from an addition, there may be mentioned, for example, the chimeric nucleic acid sequences comprising an additional heterologous part linked to one end, for example of the hybrid construct type consisting of a cDNA with which one or more introns would be associated.

The term “promoter sequence” used herein refers to a DNA regulatory region capable of binding RNA polymerase in a cell and initiating transcription of a downstream (3′ direction) coding sequence. For purposes of defining the present invention, the promoter sequence is fused at its 3′ terminus by the transcription initiation site and extends upstream (5′ direction) to include the minimum number of bases or elements necessary to initiate transcription at levels detectable above background.

A coding sequence is “under the control” of transcriptional and translational control sequences in a cell when RNA polymerase transcribes the coding sequence into mRNA, which is then trans-RNA spliced (if the coding sequence contains introns) and translated into the protein encoded by the coding sequence.

The term “homologous” in all its grammatical forms and spelling variations refers to the relationship between proteins that possess a “common evolutionary origin,” including homologous proteins from different species. Such proteins (and their encoding genes) have sequence homology, as reflected by their high degree of sequence similarity. This homology is greater than about 75%, greater than about 80%, greater than about 85%. In some cases, the homology will be greater than about 90% to 95% or 98%.

“Amino acid sequence homology” is understood to include both amino acid sequence identity and similarity. Homologous sequences share identical and/or similar amino acid residues, where similar residues are conservative substitutions for, or “allowed point mutations” of, corresponding amino acid residues in an aligned reference sequence. Thus, a candidate polypeptide sequence that shares 70% amino acid homology with a reference sequence is one in which any 70% of the aligned residues are either identical to, or are conservative substitutions of, the corresponding residues in a reference sequence.

The term “polypeptide” used herein refers to a polymeric compound comprised of covalently linked amino acid residues. Amino acids are classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group. A polypeptide of the invention preferably comprises at least about 14 amino acids.

The term “protein” used herein refers to a polypeptide which plays a structural or functional role in a living cell.

The term “corresponding to” is used herein to refer to similar or homologous sequences, whether the exact position is identical or different from the molecule to which the similarity or homology is measured. A nucleic acid or amino acid sequence alignment may include spaces. Thus, the term “corresponding to” refers to the sequence similarity, and not the numbering of the amino acid residues or nucleotide bases.

The term “derivative” used herein refers to a product comprising, for example, modifications at the level of the primary structure, such as deletions of one or more residues, substitutions of one or more residues, and/or modifications at the level of one or more residues. The number of residues affected by the modifications may be, for example, from 1, 2 or 3 to 10, 20, or 30 residues. The term derivative also comprises the molecules comprising additional internal or terminal parts, of a peptide nature or otherwise. In particular, they may be active parts, markers, amino acids, such as methionine at position −1. The term derivative also comprises the molecules comprising modifications at the level of the tertiary structure (N-terminal end, and the like). The term derivative also comprises sequences homologous to the sequence considered, derived from other cellular sources and in particular, from cells of human origin or from other organisms, and possessing activity of the same type or of substantially similar type. Such homologous sequences may be obtained by hybridization experiments. The hybridizations may be performed based on nucleic acid libraries using, as probe, the native sequence or a fragment thereof, under conventional stringency conditions or preferably under high stringency conditions.

The term “analyte” as used herein means any one of a nucleic acid, a ribonucleic acid, a polypeptide, a carbohydrate, a protein, a peptide, an amino acid, a hormone, a steroid, a vitamin, an ion, a metabolite, a chemical, an element, a derivative, an analogue, a polysaccharide, a lipid, a fatty acid, a lipopolysaccharide, a glycoprotein, a lipoprotein, a nucleoprotein, an oligonucleotide, an antibody, an immunoglobulin, a coagulation factor, a peptide hormone, a protein hormone, a non-peptide hormone, an interleukin, an interferon, a cytokine, a chemokine, a bacterial cell, an eukaryotic cell, a plant cell, a fungal cell, a protozoan cell, a cell-surface molecule, a microorganism, a small organic molecule, a virion, a bacterium, a toxin, a drug, a cell membrane, a membrane fraction, a protein complex, an antigen, a hapten, a receptor, a macromolecule, or a molecular complex comprising two or more of any of the aforementioned items.

The term “biological sample” as used herein means a sample collected from a mammalian subject and may include any one of blood, serum, milk, sweat, semen, ejaculate, mucus, tears, saliva, plasma, secretions of the genitourinary tract, lymph fluid, urine, white blood cells, pleural fluid, ascites, sputum, peritoneal fluid, cerebrospinal fluid, pleural fluid, pericardial fluid, amniotic fluid, synovial fluid, interstitial fluid, and any combinations or mixtures of the aforementioned items. It is to be noted that the term “biofluid” may be used in place of the term “biological sample”.

The term “complexing domain” used herein refers to a conserved stable part of a given protein sequence and structure that can evolve, function, and exist independently of the rest of the protein chain and in particular, to a selected protein sequence to which a fragment of a split enzyme can be fused. Examples of suitable “complexing domains” for bonding with split enzyme fragments disclosed herein include proteins, enzymes, peptide antigens, lipoprotein antigens, glycoprotein antigens, small molecule antigens, heterodimerizing proteins, homodimerizing proteins, antibody-binding proteins, aggregating proteins, antibodies, antibody derivatives, antibody mimics, antibody variants, aptamers, DNA oligomers, PNA oligomers, and the like.

The phrases “close enough proximity” and “close proximity” as used herein are descriptive of conditions wherein two fragments from a split enzyme that are separately fused to one or more complexing domains, are brought together in a suspension or solution or fluid mixture so that interactions may occur between the two fragments (also known as “complementation”) such that split enzyme functionality is restored. These phrases may also be used to refer to two fragments from a split enzyme wherein each fragment is fused to a complexing domain, and one of the fragment-complexing domain structures physically immobilized is in close enough proximity to an oxidoreductase (glucose oxidase or glucose reductase) such that chemical transfer may occur between the complemented enzyme and the oxidoreductase thereby enabling signal transduction. These phrases may also be used to refer to one of the fragments from a split enzyme that is fused to a complexing domain and an oxidoreductase, whereby the oxidoreductase is fused to a surface or an electrode, and there is electron transfer to this surface or electrode.

The term “biosensor” as used herein refers to a device or apparatus that may be used to detect an analyte in biological sample wherein the biosensor is provided with both fragments of a split enzyme wherein each fragment is separately fused to a complexing domain. When contacted by the biological sample, the analyte will be captured by binding to either immobilized or non-immobilized enzyme fragments thereby restoring enzyme function which may then be detected, for example, with an electrical assay that measures electron transfer. Alternatively, a suitable enzyme substrate may be added to the biosensor after it has been contacted by the biological sample, to detect and measure the production of one or more breakdown products by the restored enzyme activity, wherein the detection of the breakdown product may be one of a colorimetric assay, a fluorometric assay, and a luminescent assay.

According to one aspect, a suitable biosensor may comprise a paper strip or a plastic strip or a polymeric strip or other like strip onto which have been immobilized fragments from a selected split enzyme wherein the fragments have been fused to one or more complexing domains. Such biosensors may be referred to herein as a “test strip” and/or a “biosensor test strip”. In one example, such a biosensor strip may be contacted with a biological sample after which, a suitable substrate may be contacted with the test strip resulting in the formation of one or more substrate breakdown products that may be detected with a colorimetric assay, a fluorometric assay, or a luminescent assay. In another example, the biosensor test strip may additionally comprise an electrode extending along one surface of the strip to which, fragments from a selected split enzyme fused to one or more complexing domains, have been immobilized. Contacting such a biosensor with biological sample will result in electron transfer between the enzyme fragments that is measurable with a suitable instrument.

According to another aspect, a suitable biosensor is a composition comprising two or more components wherein a first component is fluid mixture of one of the two types of fragments from a split enzyme, fused to a complexing domain, and the second component is a fluid mixture of other of the two types of fragments from a split enzyme, fused to a complexing domain. The two components are mixed together with a biological sample after which, electron transfer within the mixture may be measured with a suitable instrument. Alternatively, a suitable substrate may be added to the mixture and after a selected period of time, the presence and quantity of substrate breakdown products may be measured with a colorimetric assay or a fluorometric assay or and a luminescent assay.

According to one aspect, kits according to the present disclosure may comprise, for example, one or more biosensor test strips. Such kits may additionally comprise a selected enzyme substrate. Such kits may additionally comprise one or more enzyme assay reagents for addition individually or in mixtures, to the test strips for use in certain types of colorimetric assays known to those skilled in these arts. Optionally, the kits may comprise one or more biosensor test strips that have an electrode extending along one or more surfaces of the test strip.

According to another aspect, kits according to the present disclosure may comprise one or more biosensors having at least two components wherein the first components is a fluid mixture of one of the two types of fragments from a split enzyme, fused to a complexing domain, and the second component is a fluid mixture of other of the two types of fragments from a split enzyme, fused to a complexing domain. The two components may be mixed together with a biological sample after which, electron transfer within the mixture may be measured with a suitable instrument. Alternatively, a suitable enzyme substrate may be added to the mixture of components and biological sample, and after a selected period of time, the presence and quantity of substrate breakdown products may be measured with a colorimetric assay or a fluorometric assay or and a luminescent assay. It is an option for such kits to additionally comprise a component that contains the selected enzyme substrate. It is also an option for such kits to additionally comprise one or more enzyme assay reagents mixing with the mixture of components and biological sample and/or with the mixture of components, biological sample, and selected enzyme substrate. All of the above mixtures of the components with the biological sample can be assayed directly in one of a colorimetric assay, a fluorometric assay, and a luminescent assay. Alternatively, any of the above mixtures of the components with the biological sample can be transferred to a test strip for further analysis.

### EXAMPLES

### Example 1

### 1.1 Materials

Monoclonal antibodies (anti-HIS-tag mAb and anti-HA-tag tag mAb; 1 mg/ml) were purchased from MBL Corporation, whereas rabbit polyclonal anti-HIV p24 polyclonal serum was purchased from Abcam (ab63913), and bovine IgG (12.8 mg/ml) was purchased from Sigma-Aldrich.

### 1.2 Bacterial Strains

All plasmid manipulations and amplifications were performed in E. coli strain DH5a (New England Biolabs) whereas protein expression was done in a TreA knock-out of E. coli strain BL-21 (DE3) (NEB), named BL-21ΔTreA, constructed by targeted chromosomal gene knockout system using red recombinase as taught by Murphy (2011, Targeted chromosomal gene knockout using PCR fragments. Methods Mol. Biol. 765:27-42). The entire TreA gene was replaced with Tn5 (aph) type II (kanamycin resistance) using the following primers: (i) forward: 5′-TATGGACAGCAAGCGAACCG-3′ (SEQ ID NO: 1) and (ii) reverse: 5′-TCAGAAGAACTCGTCAAGAAG-3′ (SEQ ID NO: 2). S. aureus and S. uberis isolates were provided by the Canadian Bovine Mastitis Milk Quality Research Network (CBMQRN). All strains were grown in LB broth at 37° C.

### 1.3 Plasmids

The gene for periplasmic Trehalase (TreA) was amplified from E. coli strain BL-21. During PCR amplification, the secretion peptide was replaced with a HIS-tag, and Ncol and AvrII restriction sites were introduced at 5′ and 3′ ends of the gene, respectively, to clone the amplified fragment in pETDuet vector (Novagen). Primer sequences used for generation of all constructs are shown in Table 1.

TABLE 1
Primers used in this study

| Construct | Primer | Sequence |
|---|---|---|
| pETDuet-HIS- | HIS-TreAFatg-F | TATACCATGGCACACCATCACCATCACCATGAAGAAACACCG |
| TreA | | GTAACACCACA (SEQ ID NO: 3) |
| | TreARtaa-R | TATACCTAGGTTAAGGTGTGGGTTGTGCCTCT (SEQ ID NO: |
| | | 4) |
| pETDuet-HIS-N- | HIS-TreAFatg-F | TATACCATGGCACACCATCACCATCACCATGAAGAAACACCG |
| ter | | GTAACACCACA (SEQ ID NO: 3) |
| | TreA-BamHI-N-R | TAATTCCTAGGTCAGGATCCCGGAACATATTTCTCGCCTTC |
| | | (SEQ ID NO: 5) |
| pETDuet-N-ter- | TreAFatg-F | TATACCATGGAAGAAACACCGGTAACACCA(SEQ ID NO: 6) |
| HIS | TreA-TruncN-R | TAATTCCTAGGTCAATGGTGATGGTGATGGTGCGGAACATAT |
| | | TTCTCGCCTTC (SEQ ID NO: 7) |
| pETDuet-HIS-C- | TreA-TruncC-F | TAATTCCATGGCACACCATCACCATCACCATAATTTCACCCTG |
| ter | | CCGAAAG (SEQ ID NO: 8) |
| | TreARtaa-R | TATACCTAGGTTAAGGTGTGGGTTGTGCCTCT (SEQ ID NO: |
| | | 4) |
| pETDuet-C-ter- | TreA-SaIl-C-F | TAATTCCATGGCAGTCGACAATTTCACCCTGCCGAAAG (SEQ |
| HIS | | ID NO: 9) |
| | TreA-HIS-R | TATACCTAGGTTAATGGTGATGGTGATGGTGAGGTGTGGGT |
| | | TGTGCCTCT (SEQ ID NO: 10) |
| pETDuet-HA-N- | HA-TreA-N-F | TATACCATGGCATACCCATACGATGTTCCAGATTACGCTGTCG |
| ter | | ACGAAGAAACACCGGTAACA (SEQ ID NO: 11) |
| | TreA-TruncN-R | TAATTCCTAGGTCAATGGTGATGGTGATGGTGCGGAACATAT |
| | | TTCTCGCCTTC (SEQ ID NO: 7) |
| pETDuet-N-ter- | HIS-TreAFatg-F | TATACCATGGCACACCATCACCATCACCATGAAGAAACACCG |
| HA | | GTAACACCACA (SEQ ID NO: 3) |
| | TreA-N-HA-R | TATACCTAGGTCAAGCGTAATCTGGAACATCGTATGGGTAGG |
| | | ATCCCGGAACATATTTCTCGCC (SEQ ID NO: 12) |
| pETDuet-HA-C- | HA-TreA-C-F | TATACCATGGCATACCCATACGATGTTCCAGATTACGCTGTCG |
| ter | | ACAATTTCACCCTGCCGAAA (SEQ ID NO: 13) |
| | TreA-HIS-R | TATACCTAGGTTAATGGTGATGGTGATGGTGAGGTGTGGGT |
| | | TGTGCCTCT (SEQ ID NO: 10) |
| pETDuet-C-ter- | TreA-TruncC-F | TAATTCCATGGCACACCATCACCATCACCATAATTTCACCCTG |
| HA | | CCGAAAG (SEQ ID NO: 8) |
| | TreA-C-HA-R | TATACCTAGGTCAAGCGTAATCTGGAACATCGTATGGGTAGG |
| | | ATCCAGGTGTGGGTTGTGCCTC (SEQ ID NO: 12) |

Coding sequences for HIV p24 (AC: KJ925006.1) were synthetized using GeneArt (Thermo Fisher Scientific). The coding sequences for Protein G (TYKLILNGKTLKGETTTEAVDAATAEKVFKQYANDNGVDGEWTYDAATKTFTV TE) (SEQ ID NO: 14), Protein A (TADNKFNKEQQNAFYEILHLPNLNEEQRN GFIQSLKDDPSQSANLLAEAKKLNDAQAPKA) (SEQ ID NO:15) and Protein L (AMEEVTIKANLIFANGSTQTAEFKGTFEKATSEAYAYADTLKKDNGEWTVDVADKGYTLNIKFAG) (SEQ ID NO:16) were incorporated as oligonucleotides linkers. All coding sequences introduced at the N-terminus of fragments were cloned between Ncol and Sall restriction sites, whereas coding sequences introduced at C-terminus were cloned between BamHI and AvrII.

It is to be noted that the amino acid sequence of trehalase (TreA) with a HIS-tag at the N-terminus is shown as SEQ ID NO 17. Variants with the HIS-tag at the C-terminus or at both termini, code for SEQ ID NOs: 18 and 19 respectively. TreA N (66 aa long) and C terminal (456 aa long) fragments tagged with the HIS or HA tag at the N- or C-terminals, were generated using the same strategy.

### 1.4 Protein Expression, HIS-Tag Purification and Buffer Exchange

Proteins were expressed in BL-21ΔTreA by induction with 0.5 mM of isopropyl-β-D-thiogalactoside (IPTG) (UBP Bio) for 3 h at 37° C. Bacterial lysates were prepared by harvesting 10 ml of induced bacterial colonies at 3,000×g at 4° C. for 10 min. Pellets were washed with PBS, re-suspended in 800 μl of 6M guanidinium buffer (6M Guanidinium-HCl; 25 mM Imidazole; PBS 1×) and sonicated using five 5-s bursts (total of 30 s). Protein fragments were purified on Ni-NTA columns (Fisher Thermo Scientific) according to the manufacturer's instructions. Briefly, proteins were fused on columns of equilibrated Ni-NTA resin in the presence of 6 M guanidinium buffer (6 M guanidinium-HCl; 25 mM imidazole; PBS 1×), refolded on column during washing steps with wash buffer (25 mM imidazole; PBS 1×) containing gradually decreasing guanidinium-HCl concentrations (6, 4, 3, 2, and 0 M, respectively) and eluted in elution buffer (250 mM imidazole; PBS 1×).

Subsequently, samples were dialyzed against 1 L of sodium maleate buffer (50 mM, pH 6) or PBS (pH 7) with SNAKESKIN® dialysis tubing (SNAKESKIN is a registered trademark of Pierce Chemical Company Corp., Rockford, Ill., USA) at 7 kDa MWKO for 24 h at 25° C. to remove imidazole.

Bacterial lysates and purified proteins were separated on 10% SDS-page (Bis-Tris Acrylamide gel) and stained with Bio-Safe Coomassie blue G-250 (Bio-Rad).

### 1.5 Complementation Assays

Protein concentration was determined with a QUBIT® protein assay kit (QUBIT is a registered trademark of Molecular Probes Inc., Eugene, Oreg., USA). Antibody complementation assays and Ni-NTA complementation assays were performed in sodium maleate buffer (50 mM, pH 6). Antibodies were detected in 1:1 molar ratios with reagents.

All assays were performed with 5 μg of the TreA^{C} fragment, whereas the concentration of TreA^{N} fragment was adjusted for every assay (to maintain a 1 to 1 molar ratio). Assays were performed in 60 μl of final volume. Assays were incubated with 0.25 M solution of trehalose (Sigma) at 25° C. for at pH 6 for 1 h or O/N at pH 7, as specified for each experiment.

Glucose concentrations were measured with ACCU-CHEK AVIVA® glucometer strips (ACCU-CHEK AVIVA is a registered trademark of Roche Diagnostics GmbH, Mannheim, Fed. Rep. Germany), a Benedict's reagent (Sigma) assay, or with a colorimetric enzymatic assay using glucose oxidase (0.26 U/mL; Sigma), horseradish peroxidase (0.2 U/mL; Sigma), and o-Dianisidine (0.5 mM; Sigma) in sodium maleate buffer (50 mM, pH 6). Absorbance (OD) was measured after 30 min (Ni-NTA, anti-HIS, anti-HA and Intein assays), after 1 h for Anti-HIV antibody assay, or 10 min (all other assays) of incubation at 450 nm of wavelength using an ENSPIRE® spectrophotometer (ENSPIRE is a registered trademark of PerkinElmer Singapore PTE Ltd., Singapore, Singapore) and Benedict's reagent assay (Sigma).

### 1.6 Lyophilisation of Proteins

Proteins were mixed in 1 to 1 weight ratio with BSA and, frozen at −80° C. for 30 min and then lyophilized O/N at −85° C. and 12 mT. Proteins were re-suspended in acidified sample (blood or milk). Samples were acidified by addition of 10 mg of citric acid, 61 mg of sodium citrate and 0.189 g of trehalose.

### 1.7 Biosensor Design

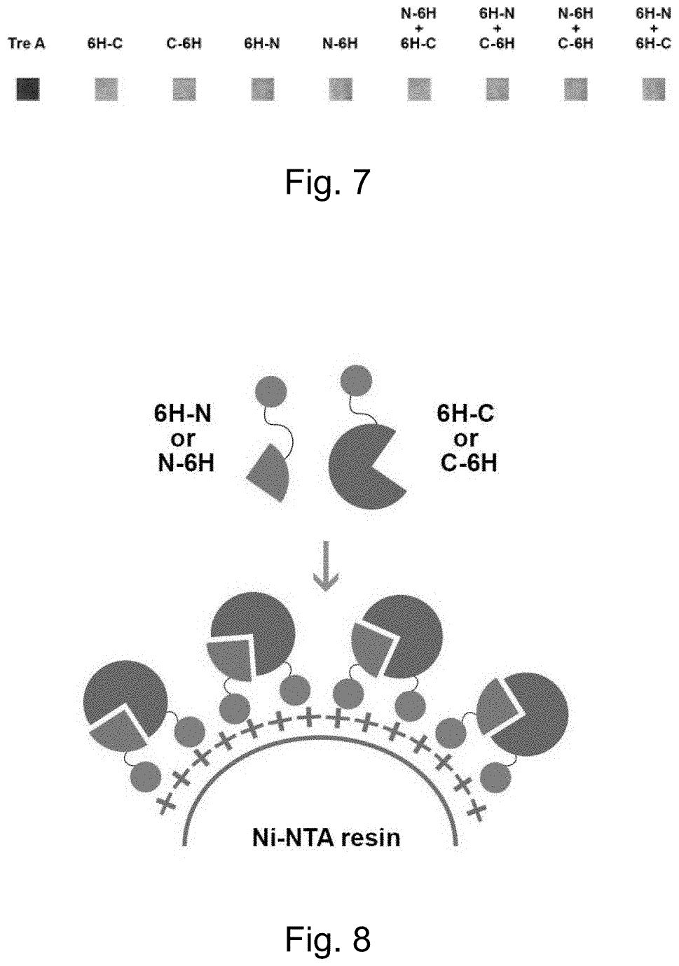

First, TreA (lacking a leader sequence) was split into two fragments: (i) 66 aa N-terminal (TreA^{N}), and (ii) 456 aa C-terminal (TreA^{C}). The fragmentation point of TreA was placed into a 12 aa long region that was unresolved in a published crystal structure (PDB: 2JF4). TreA^{N} and TreA^{C} were expressed and purified separately. Neither fragment had any detectable enzymatic activity during 24 h of incubation with substrate, nor was there evidence of self-assembly (resulting in trehalase activity) when incubated together (FIG. 7).

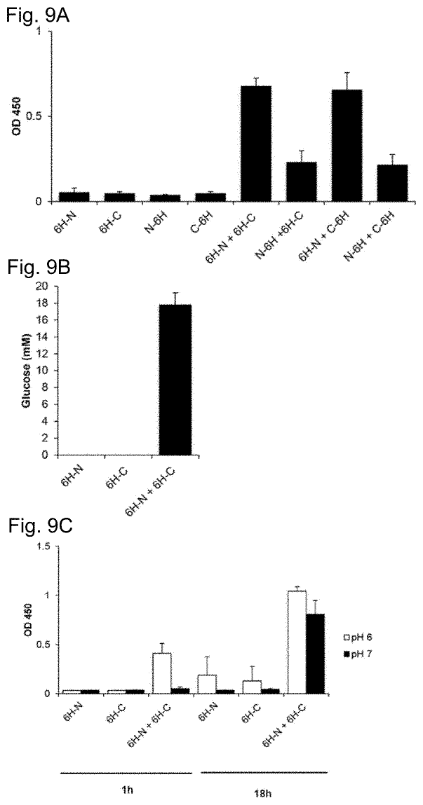

Conditional reassembly of fragments was tested by fusing HIS-tags to TreA^{N} and TreA^{C} (SEQ ID NOs: 20, 21, 22, 23) and immobilizing them separately or together on Ni-NTA resin columns (FIG. 8). Co-immobilization of fragments on resin beads was sufficient to induce complementation, regardless of the N or C terminal position of the HIS-tag on either fragment (FIGS. 9A, 9B, 9C). Trehalose was only hydrolysed when TreA^{N} and TreA^{C} fragments were fused together on a column, whereas no glucose was detected when two fragments were separately fused on a column. Elution of co-immobilized TreA fragments from the column abolished trehalase activity in the elution, confirming transient complementation.

In accordance with known optimal pH of TreA, the Ni-NTA resin complementation assay reacted faster at pH 6 then at pH 7, producing comparable amounts of glucose (output signal) in 1 h at pH 6 and 18 h at pH 7 (FIG. 9A). Therefore, analyte detection assays were performed at pH 6 (unless the sensor that was incorporated into a detection assay required a neutral pH for proper function).

### 1.8 Antibody Detection

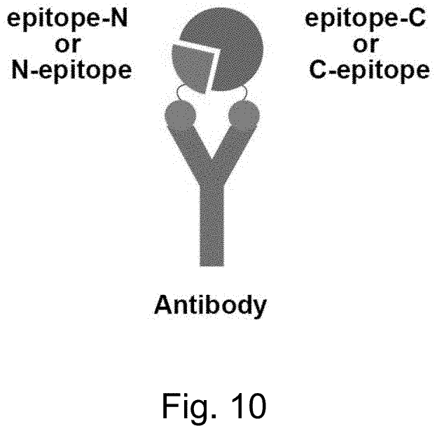

Antibody-dependent complementation of split TreA was demonstrated with anti-HIS and anti-HA monoclonal antibodies and anti-HIV (anti-p24) polyclonal serum as illustrated in FIG. 10.

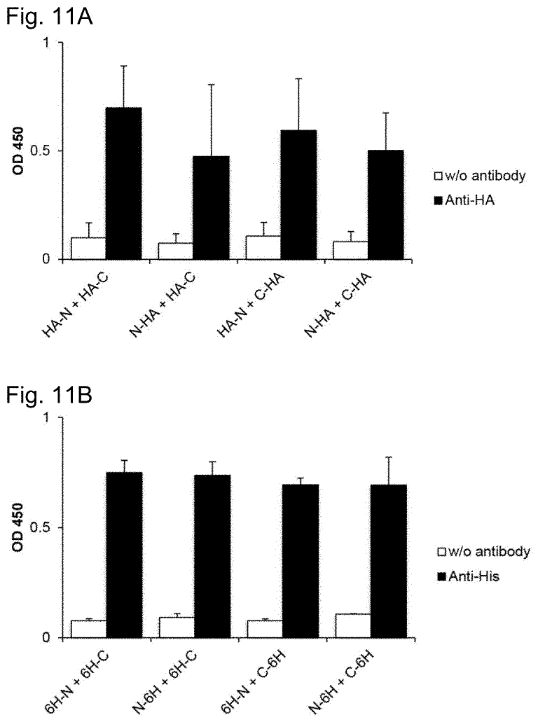

Incubation of HA-tagged TreA^{N} and TreA^{C} fragments (SEQ ID NOs: 24, 25, 26, 27) with anti-HA mAb or HIS-tagged TreA^{N} and TreA fragments (SEQ ID NO and SEQ ID NOs: 21, 22, 23, 24) with anti-HIS mAb induced complementation and subsequent trehalase activity, measured as glucose production (FIGS. 11A, 11B). The location of the HIS-tag antigen on either N or C-terminus only had minor impact on trehalase activity.

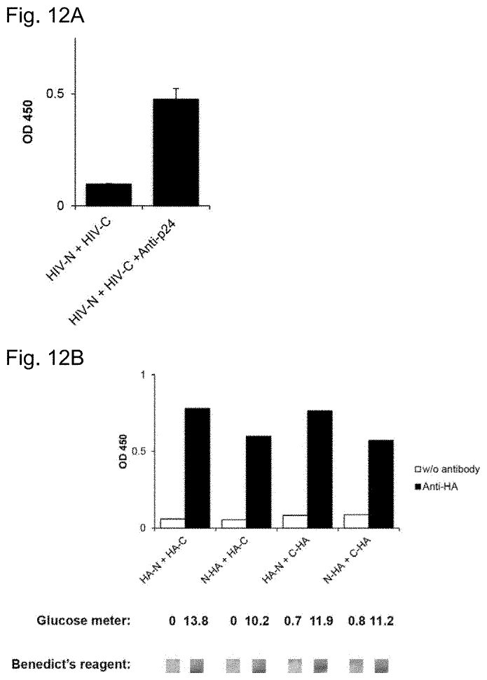

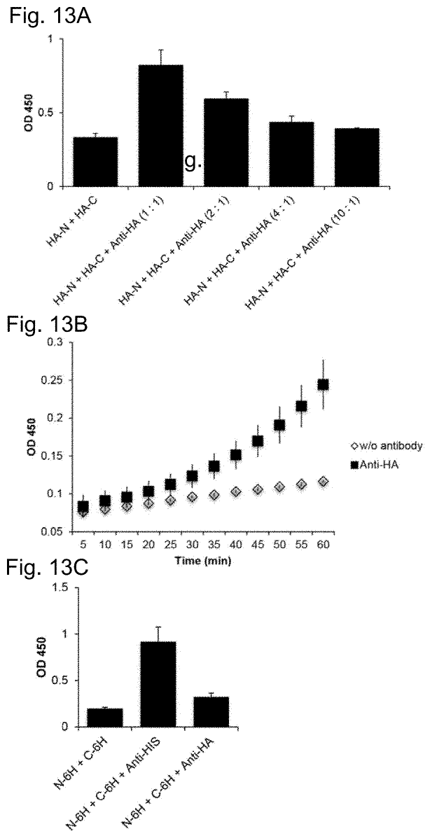

Glucose production after complementation of TreA^{N} and TreA^{C} fragments carrying the HA-tag (SEQ ID NOs: 25, 26, 27, 28) with anti-HA mAb was measured in comparison by GOx/HRP (3EA assay) or Benedict's reagent or glucometer strips (FIG. 12B). The time response of the anti-HA specific TreA detection assay was followed for 1 h. The assay was incubated with or without Anti-HA, and glucose concentrations were measured every 5 min and the increase of glucose concentration was detected after 25 min (FIG. 13B).

Next, experimental sensitivity of antibody-mediated complementation was examined. Complementation of HA tagged TreA fragments was induced with decreasing concentrations of anti-HA mAb. Output glucose signal decreased proportionally with the analyte, with complementation detected even when the concentration of anti-HA was decreased 5-fold (FIG. 13A).

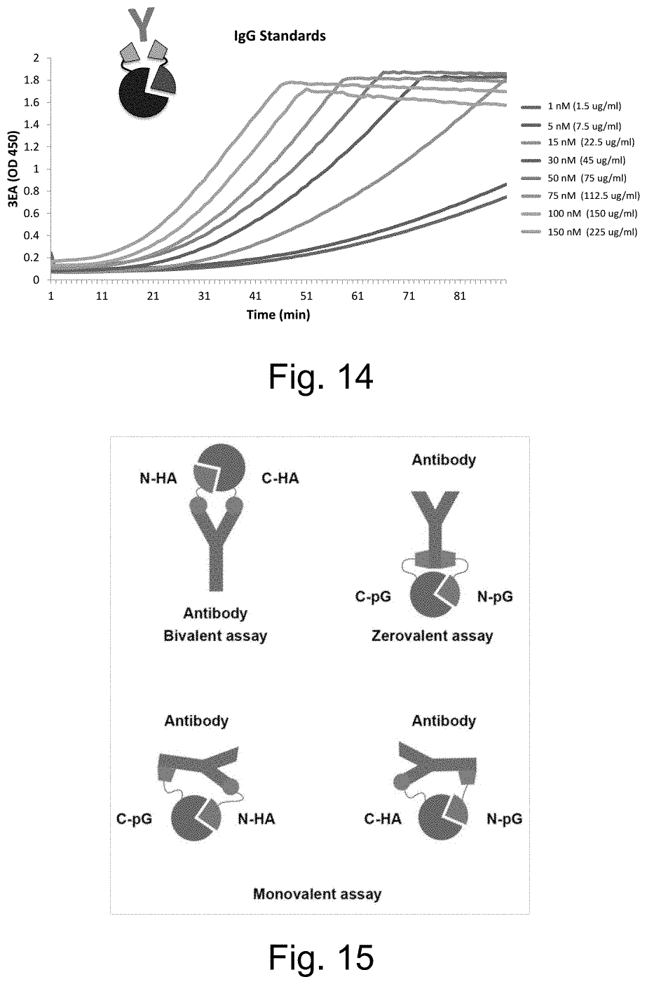

Experimental sensitivity was also demonstrated in an experiment where decreasing amounts of total bovine immunoglobulin G was detected with the TreA fragments fused to protein G (SEQ ID NOs: 28, 29). Colorimetric signals as generated by the GOx, HRP and O-dianisidine reagents as measures of glucose, were proportional with the concentrations of IgG in the samples (FIG. 14).

Specificity of antibody-mediated complementation was tested by incubating HIS-tagged fusions of TreA^{N} and TreA^{C} with non-cognate anti-HA mAbs next to cognate anti-HIS mAbs. Anti-HA mAb were unable to complement HIS-tagged TreA fragments, whereas anti-HIS mAb resulted in glucose production (FIG. 13C).

A complete antigenic protein, non-assembling mutant of HIV capsid protein p24, was fused to both TreA^{N} and TreA^{C} (SEQ ID NOs: 30, 31) to investigate complementation by antibodies in a non-purified rabbit polyclonal hyper-immune antiserum. Increased glucose concentrations were detected only in the presence of anti-p24 serum (FIG. 12A).

1.9 Detection of Antibodies Through Interactions with their Variable and Constant Regions

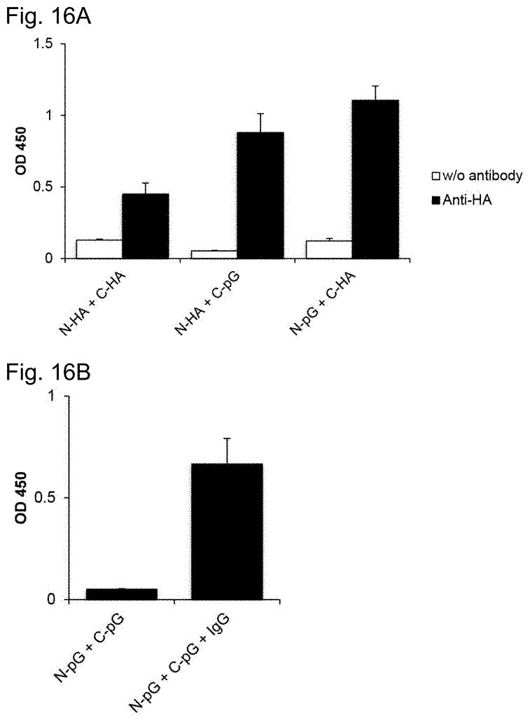

Fusions of TreA were engineered to recognise and detect various regions of antibodies (FIG. 15). Protein G (pG), a virulence factor expressed in streptococcal bacteria that binds to constant regions (Fc) of IgGs, was introduced as a sensor into TreA fusions. Combinations of TreA fragment fusions carrying protein G and HA tag (monovalent assay), Protein G only (zerovalent assay) or HA tags only (bivalent assay) were used to detect anti-HA mAb in antigen-specific or antigen non-specific manners (FIG. 16A). Fusions of TreA carrying only protein G were inefficient in detecting anti-HA mAb raised in mouse cells, although when bovine IgG were used as an analyte, dimerization was more efficient (FIG. 16B), indicating a species difference in capacity of IgG to bind two molecules of protein G.

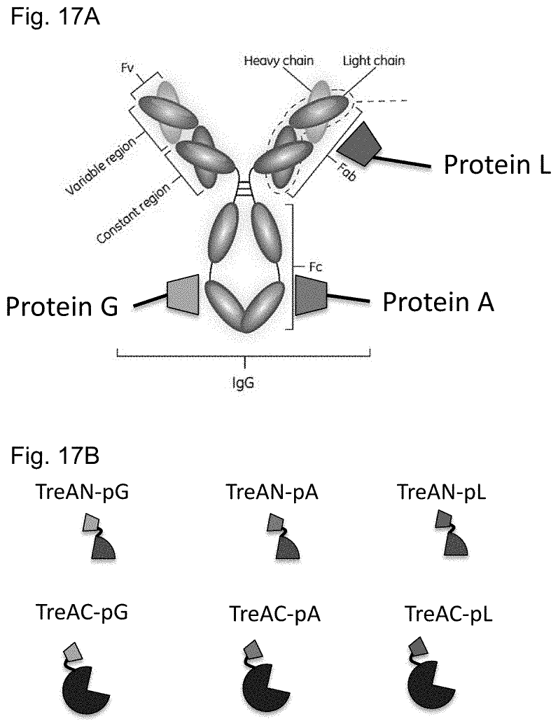

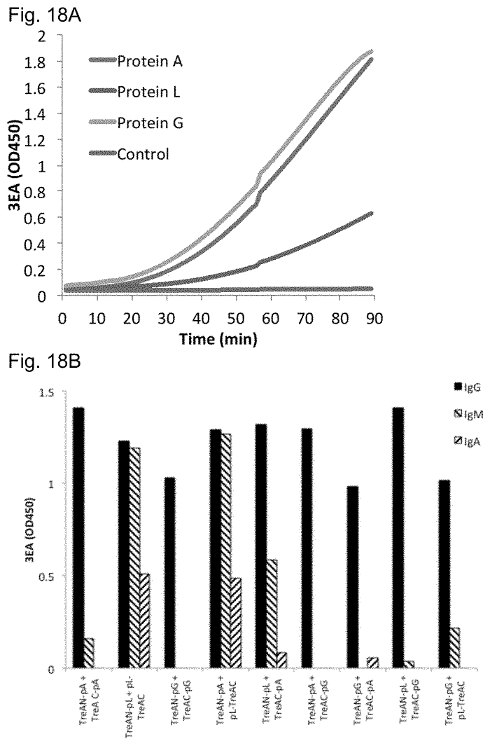

The IgG binding domain of Protein A (pA), a virulence factor expressed by staphylococcal bacteria that binds to the constant regions of (Fc) of IgGs, and the immunoglobulin binding domain of Protein L (pL), a virulence factor of Peptostreptococcus magnus that binds to the light chains of IgG, IgA and IgM, were also introduced as sensors into TreA fusions (FIGS. 17A, 17B). Combinations of TreA fragment fusions carrying pG (SEQ ID NOs: 28, 29), pA (SEQ ID NOs: 32-33), or pL (SEQ ID NOs: 34, 35), in all possible combinations, were used to detect IgG, IgM, and IgA (FIGS. 18A, 18B).

The biosensor platform tested novel split enzyme reporters by fusing polyhistidine tags (HIS) to the TreA fragment termini and immobilizing them onto a continuous binding surface of Ni-NTA resin beads. The biosensor platform disclosed herein efficiently detected monoclonal antibodies as well as polyclonal antibodies present in non-purified serum by fusing either peptide epitopes or whole-protein antigens to the split enzyme fragments. A positive signal (increased glucose concentrations) was detected in <30 min at room temperature with the GOx-HRP assay and with glucose strips, only when the bioreagents were interacting with cognate antibody. Successful conversion of the presence of antigen-specific antibody into a glucose signal (measured with a conventional glucometer) enables this biosensor platform to monitor host humoral immune responses (for example, in response to an infection).

By incorporating immunoglobulin binding proteins that recognize the constant regions of immunoglobulins, the TreA platform detects total immunoglobulin concentrations. In addition, immunoglobulin binding protein and antigen fusions were combined to detect antigen-specific antibodies in a manner that required only one TreA fragment to be modified to result in the new test disclosed herein, which can be used to simplify antigen screening.

The biosensor platform tested novel split enzyme reporters by fusing polyhistidine tags (HIS) to the TreA fragment termini and immobilizing them on a continuous binding surface of Ni-NTA resin beads. The biosensor platform disclosed herein efficiently detected monoclonal antibodies as well as polyclonal antibodies present in non-purified serum by fusing either peptide epitopes or whole-protein antigens to the split enzyme fragments. A positive signal (increased glucose concentrations) was detected in <30 min at room temperature with the GOx-HRP assay and with glucose strips, only when the bioreagents were interacting with cognate antibody. Successful conversion of the presence of antigen-specific antibody into a glucose signal (measured with a conventional glucometer) enables this biosensor platform to monitor host humoral immune responses (for example, in response to an infection).

By incorporating immunoglobulin binding proteins that recognize the constant regions of immunoglobulins, the TreA platform detects total immunoglobulin concentrations. In addition, immunoglobulin-binding protein and antigen fusions were combined to detect antigen-specific antibodies in a manner that required only one TreA fragment to be modified to result in the new test disclosed herein, which can be used to simplify antigen screening.

### Example 2

### 2.1 Bacterial Strains

All plasmid manipulations and amplifications were carried out as disclosed in Example 1. Staphylococcus aureus and Streptococcus uberis strains were kindly provided by the Canadian Bovine Mastitis Milk Quality Research Network. Listeria monocytogenes strains NF-924, NF-1166, NF-1177 were kindly provided by Dr. Nancy Freitag, University of Illinois. All strains were grown in LB broth except L. monocytogenes, which were grown in BHI broth at 37° C. overnight.

### 2.2 Plasmids

Plasmids were constructed as disclosed in Example 1.

The coding sequence for bankvole PrP (residues 23 to 230; Accession No. AF367624) was synthetized using GeneArt (Thermo Fisher Scientific). The coding sequences for S. aureus binding peptide SA5-1 (VPHNPGLISLQG; SEQ ID NO: 36), for Mycobacterium avium subsp. paratuberculosis peptide Mp3 (NYVIHDVPRHPA; SEQ ID NO: 37), the complementary coiled-coil peptides with the leucine zipper motifs Ei (EIAALEKEIAALEKENAALEWEIAALEK) (SEQ ID NO: 38), Ki (KIAALKEKIAALKEKNAALKWKIAALKE) (SEQ ID NO: 39), Protein G (TYKLILNGKTLKGETTTEAVDAATAEKVFKQYANDNGVDGEWTYDAATKTFTV TE) (SEQ ID NO: 14), Protein A (TADNKFNKEQQNAFYEILHLPNLNEEQRN GFIQSLKDDPSQSANLLAEAKKLNDAQAPKA) (SEQ ID NO: 15) and Protein L (AMEEVTIKANLIFANGSTQTAEFKGTFEKATSEAYAYADTLKKDNGEWTVDVADKGYTLNIKFAG) (SEQ ID NO: 16) were incorporated as oligonucleotides linkers. All coding sequences introduced at the N-terminus of fragments were cloned between Ncol and Sall restriction sites, whereas coding sequences introduced at C-terminus were cloned between BamHI and AvrII.

### 2.3 Protein Expression, HIS-Tag Purification and Buffer Exchange

Proteins were expressed and purified as disclosed in Example 1.

For protein aggregation assays, rPrP fusions (rPrP-TreA (SEQ ID NO 44), rPrP-TreA^{N} (SEQ ID NO 45) and rPrP-TreA^{C} (SEQ ID NO 46)) were purified separately on Ni-NTA columns under denaturing conditions, then mixed (1 to 1 ratio) and co-dialyzed against 1 L of 10 mM sodium phosphate (pH 5.8) with SNAKESKIN® dialysis tubing at 7 kDa MWKO for 24 h at 25° C. to remove the denaturing agent. After dialysis, samples were centrifuged (10,000×g for 10 min) to separate precipitated and soluble proteins. Supernatant was discarded and the pellet washed with PBS and then re-suspended in 0.25 M trehalose.

### 2.4 Complementation Assays

Complementation assays were carried out as disclosed in Example 1.

Bacterial complementation assays were performed in PBS (pH 7). For this, 0.2 ml of bacterial culture grown overnight was pelleted (OD₆₀₀ₙₘ=1.2), washed 3 times with PBS, re-suspended in 10 μl of PBS (pH 7) and added to protein fragments.

For protein aggregation assays, rPrP fusions (rPrP-TreA^{N} and rPrP-TreA^{C}; SEQ ID NOs: 45, 46) were purified separately on Ni-NTA columns under denaturing conditions, then mixed (1:1 ratio) and co-dialyzed against 1 L of 10 mM sodium phosphate (pH 5.8) with SNAKESKIN® dialysis tubing at 7 kDa MWKO for 24 h at 25° C. to remove the denaturing agent. After dialysis, samples were centrifuged (10,000×g for 10 min) to separate precipitated and soluble proteins. Supernatant was discarded and the pellet washed with PBS and re-suspended in 0.25 M trehalose.

Glucose concentrations were measured as disclosed in example 1.

### 2.5 Direct Whole-Pathogen Detection

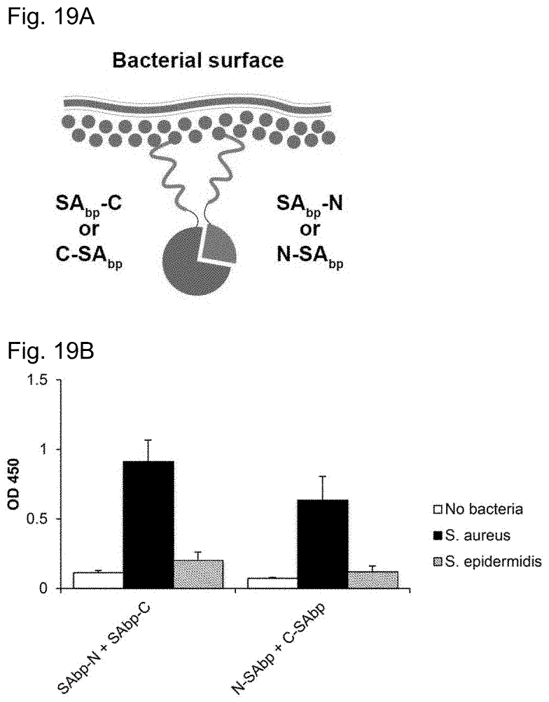

Split TreA was applied to detect intact bacterial cells by incorporating peptide aptamers (FIG. 19A). Specifically, S. aureus surface binding peptides were placed at the N or C terminals of TreA^{N} and TreA^{C} fragments (SEQ ID NOs: 40, 41, 42, 43). Resulting TreA fusions were incubated with S. aureus or S. epidermidis. Trehalase complementation and a subsequent glucose increase was only detected when TreA fragments were incubated with S. aureus, whereas glucose concentrations were low when fusions were incubated with S. epidermidis (FIG. 19B). Trehalase activity was higher when the peptide aptamer was fused to the N-terminus of both TreA fragments.

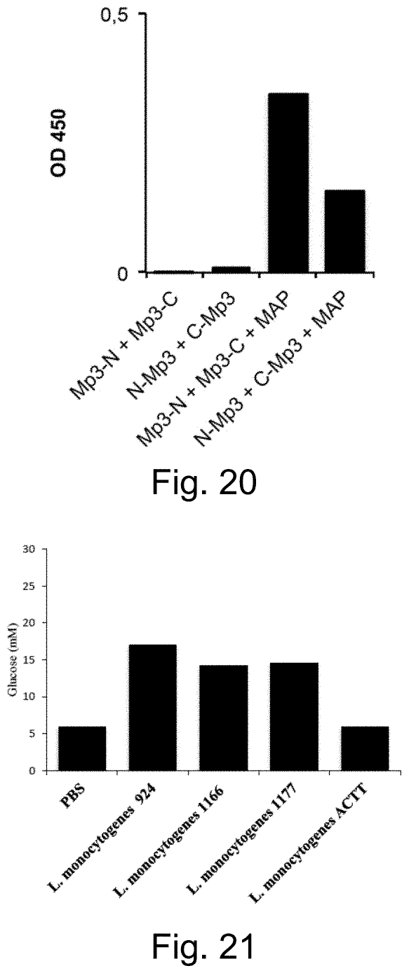

Mycobacterium avium subsp. paratuberculosis (MAP) binding peptide (Mp3) were also placed at the N or C terminals of TreA^{N} and TreA^{C} fragments (SEQ ID NOs: 44, 45, 46, 47). Resulting TreA fusions were incubated with MAP. Trehalase complementation and a subsequent glucose increase indicated the detection of MAP cells regardless of fusion of the peptide aptamers to the N or C terminus of the TreA fragments (FIG. 20).

Single-chain variable fragment (scFv), an antibody mimic that specifically recognizes ActA, a virulence factor expressed on the surface of L. monocytogenes, was fused to Tre, TreA^{N} and TreA^{C} (SEQ ID NOs: 48, 49, 50). ActA-overexpressing L. monocytogenes mutants (924, 1166, 1177) were used for comparison with wild type L. monocytogenes (ATCC) which expresses very low levels (˜up to 200-fold less) of ActA when grown extracellularly. Glucose production corresponded with expected levels of ActA expression on the bacterial cell surface (FIG. 21).

### 2.6 Small Molecule Detection

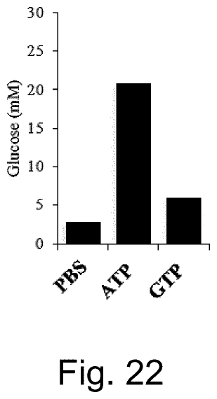

Small molecules that are known to dimerize their receptor proteins, such as ATP and estradiol, were used to complement the split TreA reporter. TreA^{N} and TreA^{C} were fused to ATPase subunit F0F1 ε from Bacillus PS3 (SEQ ID NOs: 51, 52). Protein preparations were incubated in equimolar concentration with 10 mM of ATP or 10 mM of GTP in presence of 10 mM of MgCl₂. Glucose production was observed in presence of ATP as expected in contrast to a PBS control. A more modest production was also observed in the presence of GTP (FIG. 22).

### 2.7 Protein-Protein Interaction and Protein Aggregation Detection

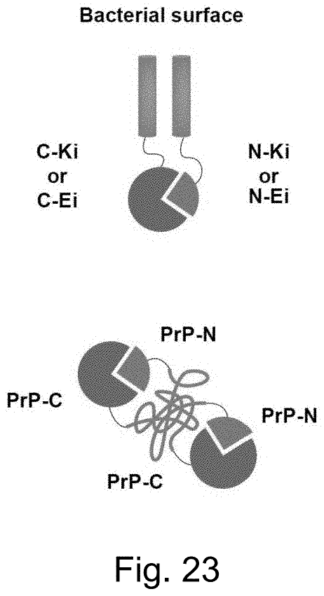

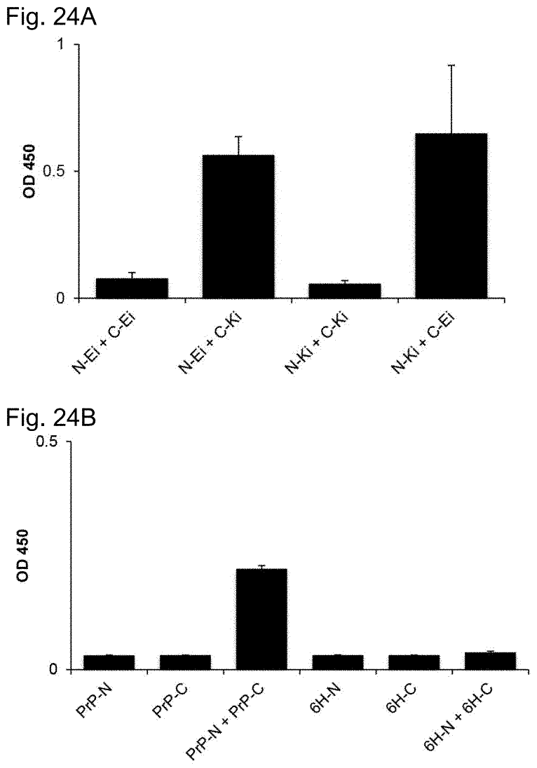

The TreA detection assay was also used to detect protein-protein interactions and protein aggregation (FIG. 23). As an example of the former, complementary coiled-coil peptides with the leucine zipper motifs Ei (SEQ ID NO: 38) and Ki (SEQ ID NO: 39) were used to induce TreA complementation. TreA^{N} and TreA^{C} with complementary heterodimzerizing peptides KI and EI or non-complementary zippers (Ei+Ei and Ki+Ki) (SEQ ID NOs: 53, 54, 56) were assayed for TreA complementation. Only combinations with complementary leucine zippers (Ki+Ei) increased glucose concentrations, whereas other combinations did not (FIG. 24A).

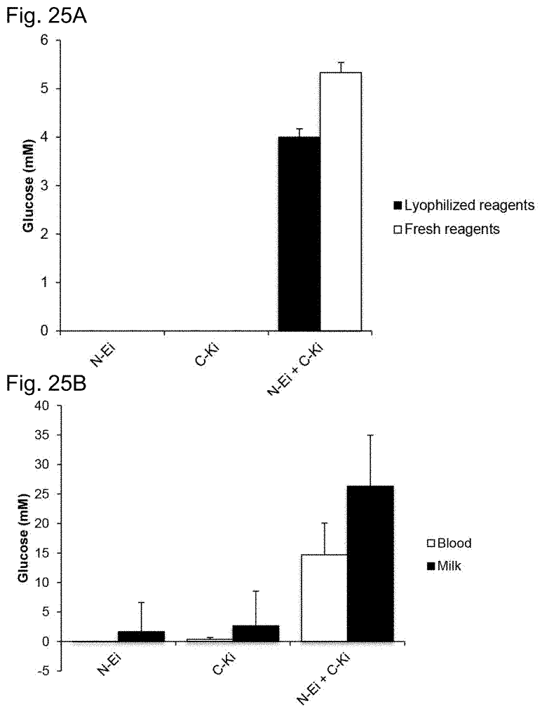

Leucine zipper TreA fusions were used as models to investigate sample matrix effects on TreA complementation assays and to explore the possibility of the bio-reagents to withstand lyophilisation and subsequent resolubization (i.e., assess stability for long-term shelf life). The TreA^{N}-Ei and TreA^{C}-Ki fusions were purified and lyophilized separately in the presence of substrate. Next, fusions were re-suspended in whole bovine blood or bovine milk (acidified to pH 6 by addition of citric acid/sodium citrate powder). Glucose concentrations were measured over 3 h; the glucose signal doubled in samples containing complementary fusions, demonstrating that neither lyophilisation nor the presence of blood or milk inhibited TreA complementation (FIGS. 25A, 25B).

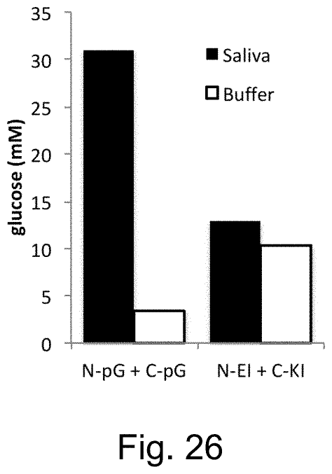

Leucine zipper TreA fusions were also tested in pure saliva samples by desolving lyophilized assay reagents in saliva. The comparison with a buffer control demonstrates that the assay reagents are equally active in saliva as in buffer (FIG. 26). Similarly, lyophilized protein G TreA fusions described in more detail in Example 1, were also reconstituted in saliva to detect the presence of IgG in saliva in comparison with buffer which did not contain antibodies (FIG. 26).

Furthermore, Tre, TreA^{N} and TreA^{C} were fused N-terminally to recombinant bank vole prion protein (rPrP^{C}) (SEQ ID Nos: 57, 58, 59), which forms aggregates spontaneously under specific conditions. The rPrP-dependent aggregation was induced by co-dialysis of TreA^{N} and TreA^{C} fusions, and protein aggregates were isolated from soluble fusion proteins and incubated with substrate. Glucose production was detected only when the TreA^{N} and TreA^{C} rPrP fusions were co-aggregated, but not when they aggregated separately (FIG. 24B). Furthermore, HIS-tagged TreA fusions showed no evidence of aggregation under the same conditions.

### 2.8 Complementation of TreA by Inteins

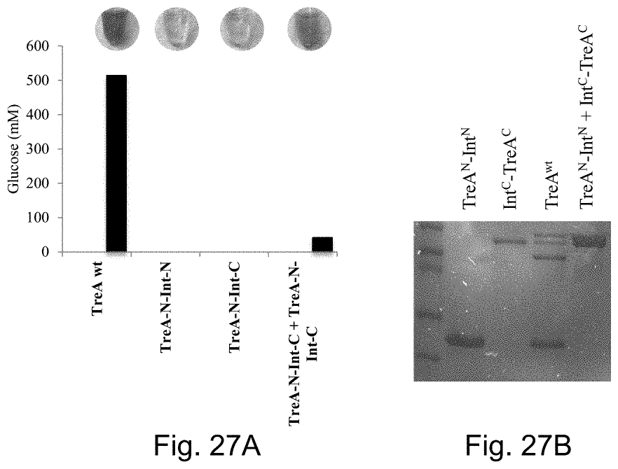

Split inteins were used to induce the complementation of TreA. Inteins are protein elements placed inside host proteins (i.e., exteins) that are able to self-excise from the extein when activated. During the excision, inteins catalyze the restoration of the peptide bond between two terminals of the original host protein, which leads to restoration of the extein and activation of its function. The CWE residues that needed to be introduced to flank the N-terminus of the C-terminal extein were also introduced in the linker region of the complete TreA protein and this scar was demonstrated not to abolish the trehalase activity in the recombinant protein (SEQ ID NO: 60). Trehalase activity was detected after mixing TreA^{N} and TreA^{C} fusions with split DnaE inteins (SEQ ID NOs: 61, 62) (FIG. 27A) and successful intein excision and TreA ligation was verified on SDS page (FIG. 27B).

This example discloses a versatile biosensor platform and demonstrates that this platform can be used to detect various types of molecules, for example antibodies (as demonstrated in Example 1), small molecules (ATP), whole pathogens and inter-molecule interactions (protein-protein binding and protein aggregation). Cytokines (for example, interferon gamma) and hormones (for example, estradiol) could also be detected with sensor proteins that dimerize in the presence with these molecules, for example with human estradiol receptor or bovine interferon-gamma receptor (SEQ ID NOs: 63, 64, 65, 66). The biosensor platform is based on split enzyme complementation of an E. coli glycolytic enzyme trehalase (TreA) that hydrolyses trehalose into two molecules of glucose, which can be easily detected with commercially available glucometers. Glucose detection is compatible with clinical samples and does not need additional handling or processing. In contrast, the output signals generated by many other approaches are less compatible with existing detectors and with detection in biofluids from humans or animals (for example, fluorescence or luminescence from GFP or luciferase).

This biosensor platform was adapted to detect whole bacterial cells, protein-protein interactions and protein aggregation, simply by replacing the sensor component with protein elements specific to the analyte of interest. Fusions of TreA fragments with small peptide aptamers specific to components present on bacterial surface or single chain fragment variables (antibody derivative) specific for surface antigen enabled detection of bacterial cells (for example, S. aureus and Listeria monocytogenes). Heterodimerizing leucine zippers and PrP were used as models to demonstrate that this platform can be applied to monitor protein-protein interactions or protein aggregation, respectively.

To summarize, disclosed herein are the implementation of dimerization and complexation strategies to complement a split enzyme to monitor various types of analytes and interactions in parallel. Also disclosed herein are complementation strategies never before combined with split reporter enzymes, for example surface binding and protein aggregation (e.g., prion protein; PrP) and amyloid p (SEQ ID NOs: 67, 68, 69). In addition, heterodimerizing peptides were used to investigate effects of clinical sample composition. It was noteworthy that that TreA complementation was not severely impacted by the composition of real life/clinical samples, in contrast to some other reporters (for example, GFP and Luciferase).

### Example 3

### 3.1 Materials

Purified Bovine IgG (12.8 mg/ml) used for standard curves was purchased from Sigma-Aldrich. Plasmids used in this example were prepared as described in Examples 1 and 2. In short, the gene coding for E. coli glycolytic enzyme TreA was split in 2 fragments, TreA N (66 aa long) and TreA C (456 aa long) through PCR amplification. Each fragment was fused C terminally to Protein G (residues: 270-324; AC: P19909) and cloned in pETDuet expression vector (Novagen, Canada) using Ncol and AvrII restriction sites.

### 3.2 Protein Purification and Lyophilisation

Proteins were recombinantly expressed in BL-21 ΔTreA strain and purified on Ni-NTA resin following the methods disclosed in Example 1. Briefly, recombinant cultures were induced with 0.5 mM of IPTG and harvested after 3 h at 37° C. Bacterial pellets with recombinant proteins were resuspended in 6M guanidinium buffer, sonicated and loaded on equilibrated Ni-NTA resin. Proteins were refolded on resin during washing steps containing gradually decreasing guanidinium-HCl concentrations and eluted in Elution buffer containing 250 mM of Imidazole. Finally, samples were dialyzed against 1 L of sodium maleate buffer (50 mM, pH 6) with SNAKESKIN® dialysis tubing for 24 h at 25° C. and protein concentration was determined with a QUBIT® assay. Lyophilized reagents were prepared by mixing the proteins in 1:1 weight ratio with BSA, frozen in microtiter plate wells at −80° C. and then lyophilized O/N at −85° C. and 12 mT.

### 3.3 Split Trehalase Immunoglobulin G Assay (STIGA)

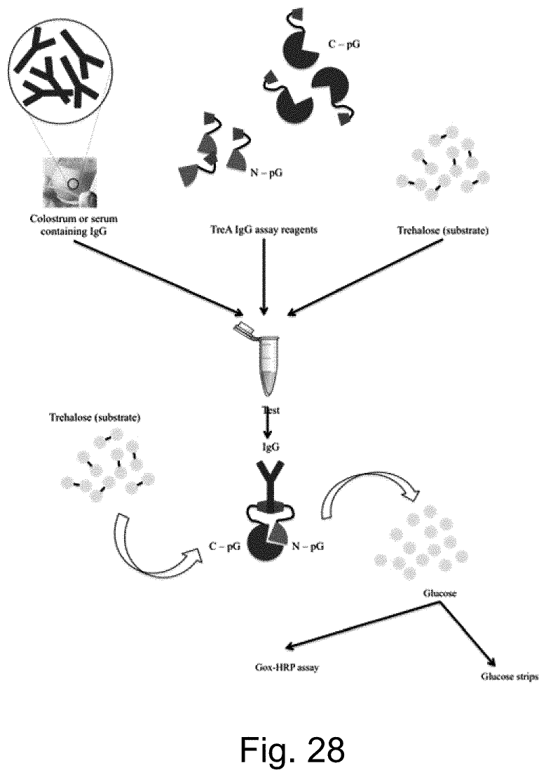

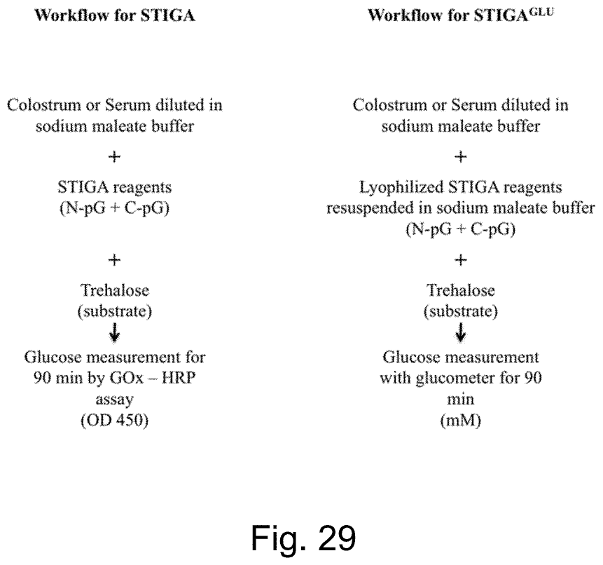

Glucose quantification was done in two ways in this example. First, glucose measurements used for statistical analysis were collected with a GOx-HRP colorimetric assay based on based on glucose oxidase (0.1 mg/mL), horse radish proxidase (0.2 U/mL), and O-dianisidine (0.5 mM) in sodium maleate buffer (50 mM) (FIG. 28). Second, STIGA quantification assays were performed in two ways (FIG. 29). The STIGA assays are based on the E. coli glycolytic enzyme, trehalase (TreA) that converts trehalose into glucose. TreA is split in two non-functional fragments i.e., (i) TreA N (N), and (ii) TreA C (C). Both functional fragments were fused C-terminally to Protein G (pG) originating from Lancefield group C and D streptococci, thereby resulting in the recombinant fusion proteins N-pG and C-pG (SEQ ID NO 28, 29). Protein G binds specifically to constant region of IgG (Fc) and consequently acts as a sensor for immunoglobulins (IgGs) independently of their affinity/antigen binding specificity. When the two fusions are incubated with samples containing IgGs (e.g. colostrum or serum), the two fusion proteins will bind to the Fc of IgG thereby leading to the dimerization and re-activation of the TreA enzyme. Re-activated TreA will produce glucose in the presence of trehalose.

GOx-HRP colorimetric STIGA: Colostrum and calf serum samples were diluted in sodium maleate buffer (50 mM, pH 6) (dairy colostrum 1:2,000; beef colostrum 1:4,000; dairy and beef calf serum 1:1,000). STIGA was performed with 20 μg of C-pG and 5.2 μg of N-pG (1:1 molar ratio) for colostrum or 10 μg of C-pG and 2.6 μg of N-pG for serum in sodium maleate buffer with 250 mM of trehalose (Sigma) in final volume of 150 μl. The glucose concentration was measured with a colorimetric enzymatic assay based on glucose oxidase (0.1 mg/mL), horseradish peroxidase (0.2 U/mL), and O-dianisidine (0.5 mM) in sodium maleate buffer (50 mM, pH 6). Absorbance (OD) was measured every minute for 90 min in an ENSPIRE® multimode plate reader at 450 nm (Perkin Elmer).

Glucose strips based STIGA (STIGA^{GLU}): This assay was performed with lyophilized protein preparations. Lyophilized proteins were resuspended in 150 μl of Sodium-maleate buffer with 250 mM of trehalose containing the same colostrum or serum dilution used in previous assay. Glucose production was measured by ACCU-CHEK AVIVA® Blood Glucose Meter every 30 min for a total of 90 min.

### 3.4 Sample Collection

Dairy colostrum (n=60) and serum samples (n=83) were randomly selected from previously collected samples. Briefly, colostrum samples were collected from 13 farms in central Alberta between February and July of 2012. Samples were collected by the farm owners, frozen at −20° C. and transported to the University of Calgary where they were stored at −80° C. Blood samples were collected at the same time from bull calves and heifer calves that were more than 24 h old and 8 days or less of age. The samples were stored on ice, transported to the University of Calgary where the serum was harvested by centrifugation at 1,800×g at 4° C. for 25 min. Serum samples were stored at −80° C. as well. Beef colostrum (n=64) and serum samples (n=84) were collected during calving seasons during 2013 through 2015 from two large commercial cow-calf operations in Alberta. IgG concentrations of all the samples were determined by Prairie Diagnostic Laboratories (University of Saskatchewan, Saskatoon, SK) by RID.

### 3.5 Statistical Analysis

Pearson correlation coefficient was used to establish association between IgG concentration measured with STIGA detection assay and RID. Sensitivity, specificity, positive and negative predicted values and accuracy were calculated using RID as the gold standard.

### 3.6 Detection of IgGs in Bovine Colostrum

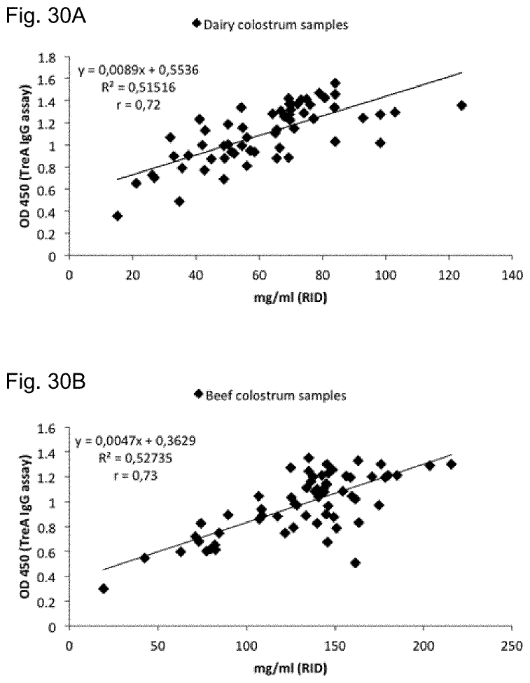

The IgG contents of dairy (n=60) and beef (n=64) colostrum samples were determined with the colorimetric STIGA. Obtained OD values corresponding to IgG levels were then correlated with IgG concentrations determined previously by RID analysis. The correlation coefficient for dairy colostrum was 0.72 (FIG. 30A), whereas for beef colostrum, the correlation coefficient was 0.73 (FIG. 30B).

With 50 mg/ml set as a threshold for adequate quality dairy colostrum, STIGA had the highest sensitivity (64.7%) and specificity (93%) when an OD of 0.9 was used as the cut-off (Table 2). Dairy samples had a 71.4% chance of being truly poor quality (=positive predictive value (PPV)) and 84.8% chance of being truly adequate (=negative predictive value (NPV)). STIGA identified 23% of analyzed colostrum samples to be of poor quality whereas RID identified 28.3% of samples of poor quality.

With a threshold set at 100 mg/ml for beef colostrum, STIGA reached its highest sensitivity (83.3%) and specificity (90.3%) when an OD of 0.8 was used as the cut-off value (Table 2). At this cut-off value, the beef colostrum sample had 66.7% PPV and a 95.92% NPV. STIGA identified 23.4% of colostrum samples of poor quality whereas according to RID 18.75% of samples were of poor quality.

### 3.7 Detection of IgGs in Calf Serum

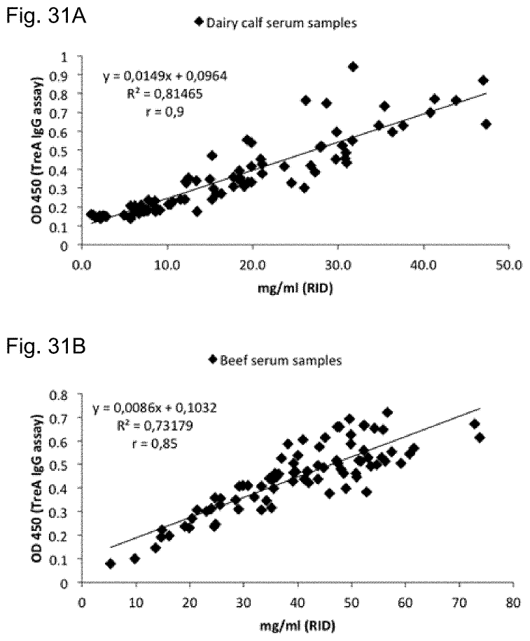

Dairy (n=83) and beef (n=84) calf sera were analyzed for their IgG concentration by STIGA. The IgG concentrations in these sera were measured previously by RID analysis. Correlation of the IgG concentrations determined by RID with OD values obtained by STIGA was 0.72 for dairy samples (FIG. 31A) and 0.85 for beef samples (FIG. 31B).

TABLE 2
Accuracy, sensitivity (Se), specificity (Sp), positive predictive
value (PPV) and negative predictive value (NPV) calculated for OD
cut points obtained with STIGA compared with 50 mg/mL IgG for dairy
colostrum and 100 mg/mL of beef colostrum determined by RID.

| | Cut point | Accuracy | Se | Sp | PPV | NPV |
|---|---|---|---|---|---|---|
| | (OD 450) | (%) | (%) | (%) | (%) | (%) |
| Dairy | 0.6 | 75.0 | 11.8 | 100.0 | 100.0 | 74.1 |
| colostrum | 0.7 | 78.3 | 23.5 | 100.0 | 100.0 | 76.8 |
| | 0.8 | 85.0 | 47.1 | 100.0 | 100.0 | 82.7 |
| | 0.9 | 85.0 | 64.7 | 93.0 | 78.6 | 87.0 |
| | 1.0 | 78.3 | 76.5 | 79.1 | 59.1 | 89.5 |
| | 1.1 | 75.0 | 88.2 | 69.8 | 53.6 | 93.8 |
| | 1.2 | 68.3 | 94.1 | 58.1 | 47.1 | 96.2 |
| Beef | 0.6 | 84.4 | 25.0 | 98.1 | 75.0 | 85.0 |
| colostrum | 0.7 | 90.6 | 66.7 | 96.2 | 80.0 | 92.6 |
| | 0.8 | 89.1 | 83.3 | 90.4 | 66.7 | 95.9 |
| | 0.9 | 79.7 | 100.0 | 75.0 | 48.0 | 100.0 |
| | 1.0 | 71.9 | 100.0 | 65.4 | 40.0 | 100.0 |
| | 1.1 | 53.1 | 100.0 | 42.3 | 28.6 | 100.0 |
| | 1.2 | 42.2 | 100.0 | 28.8 | 24.5 | 100.0 |

With thresholds for FPS set at 10 mg/ml for dairy calves and 24 mg/ml for beef calves, STIGA reached its highest sensitivity (88.9%) and specificity (85.7%) at an OD of 0.6 (Table 3) in dairy calf serum samples whereas in beef calf sera, the highest sensitivity (69.2%) and specificity (97.2%) were reached at an OD of 0.3 (Table 3). In dairy and beef calf serum samples, STIGA had 75% and 81.8% chance, respectively, of truly indicating FPS whereas it had 94% and 94.5% chance, respectively, of truly showing that the calf received enough immunoglobulins. FTP was diagnosed in 38.5% of dairy calf serum samples and 13% of beef calf serum samples by STIGA as compared to 32.5% and 15.5% respectively diagnosed by RID.

TABLE 3
Accuracy, sensitivity (Se), specificity (Sp), positive predictive
value (PPV), and negative predictive value (NPV) calculated for
OD cut points obtained with STIGA compared with 10 mg/mL IgG for
dairy calf sera and 24 mg/mL for beef calf sera determined by RID.

| | Cut point | Accuracy | Se | Sp | PPV | NPV |
|---|---|---|---|---|---|---|
| | (OD 450) | (%) | (%) | (%) | (%) | (%) |
| Dairy | 0.2 | 91.6 | 77.8 | 98.2 | 95.5 | 90.2 |
| calf | 0.3 | 86.7 | 100.0 | 80.4 | 71.1 | 100.0 |
| sera | 0.4 | 67.5 | 100.0 | 51.8 | 50.0 | 100.0 |
| | 0.5 | 55.4 | 100.0 | 33.9 | 42.2 | 100.0 |
| | 0.6 | 45.8 | 100.0 | 19.6 | 37.5 | 100.0 |
| | 0.7 | 41.0 | 100.0 | 12.5 | 35.5 | 100.0 |
| | 0.8 | 34.9 | 100.0 | 3.6 | 33.3 | 100.0 |
| Beef | 0.1 | 85.7 | 7.7 | 100.0 | 100.0 | 85.5 |
| calf | 0.2 | 90.5 | 38.5 | 100.0 | 100.0 | 102.9 |
| sera | 0.3 | 92.9 | 69.2 | 97.2 | 81.8 | 94.5 |
| | 0.4 | 83.9 | 100.0 | 91.5 | 46.4 | 116.1 |
| | 0.5 | 52.4 | 100.0 | 43.7 | 24.5 | 100.0 |
| | 0.6 | 31.0 | 100.0 | 18.3 | 18.3 | 100.0 |
| | 0.7 | 16.7 | 100.0 | 1.4 | 15.7 | 100.0 |

### 3.8 Detection of IgGs by STIGA Using a Glucometer (STIGA^{glu})

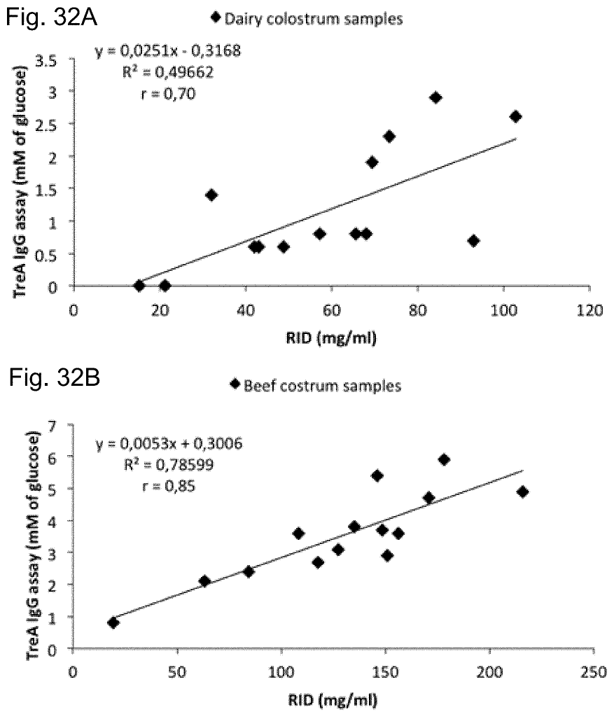

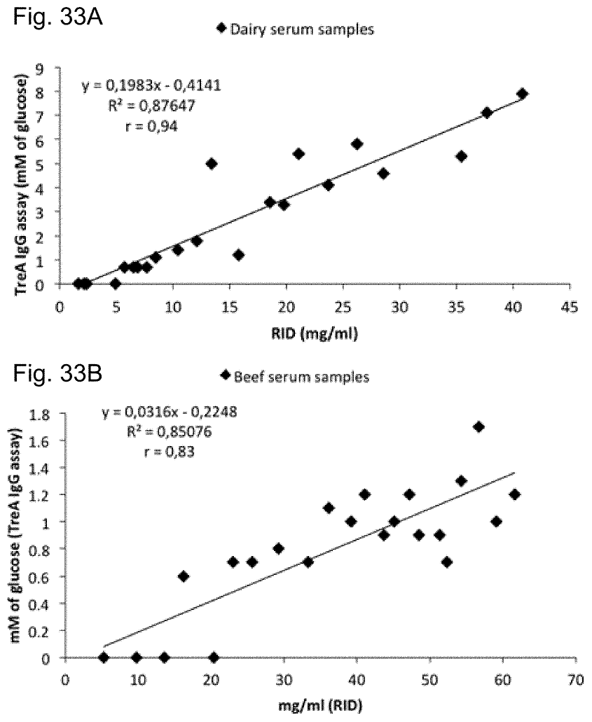

In order to develop STIGA for future on-farm use, a smaller sample set for each test group (dairy colostrum n=14; beef colostrum n=14; dairy calf sera n=18 and beef calf sera n=18) was analyzed with lyophilized STIGA bioreagents, and the produced glucose was measured with a common glucometer. The correlation coefficients between RID determined IgG concentrations and glucose levels were 0.7 for dairy colostrum (FIG. 32A), 0.85 for beef colostrum (FIG. 32B), 0.94 for dairy calf sera (FIG. 33A), and 0.83 for beef calf sera (FIG. 33B).

### Example 4

### 4.1 Bacterial Strains

All plasmid manipulations and amplifications were performed as disclosed in Example 1.

### 4.2 Plasmids

Coding sequences for PQQ-GDH from Alcinetobacter calcoaceticus (PDB: 1CQ1) was synthetized using GeneArt (Thermo Fisher Scientific). This sequence plus a sequence for a short flexible linker and a His-tag were introduced at the N-terminus of the TreA^{N} fragment (or TreA SEQ ID NO: 70) that was either fused with leucine zipper peptides EI or KI (SEQ ID NOs: 71, 72), were cloned between Ncol and Sall restriction sites.

### 4.3 Protein Expression, HIS-Tag Purification and Buffer Exchange

Protein expression and purification were carried out as disclosed in Example 1.

### 4.4 Complementation and Colorimetric Assays

Protein concentration was determined with a QUBIT® protein assay kit. Complementation of TreA fragments fused to leucine zipper peptides (SEQ ID NOs: 55, 56) was measured by a GDH assay. Complementation of the TreA fragments leads to trehalase activity and consequently glucose production. The glucose is hydrolysed by the fused GDH which leads to a colorimetric detection in the presence of the GDH assay reagents (3.0 mM 1-methoxy-5-methylphenazinium methyl sulfate, 6.6 mM Thiazolyl Blue Tetrazolium Bromide, 1 mM Pyrroloquinoline quinone or Methoxatin disodium salt (PQQ), 25 mM CaCl2, 10% Triton-100, 1 M Trehalose, 200 mM sodium maleate (pH 6.0). The reaction was read at OD at 570 nm every 1 min for 90 minutes.

### 4.5. Complementation of TreA Fragments Fused to GDH

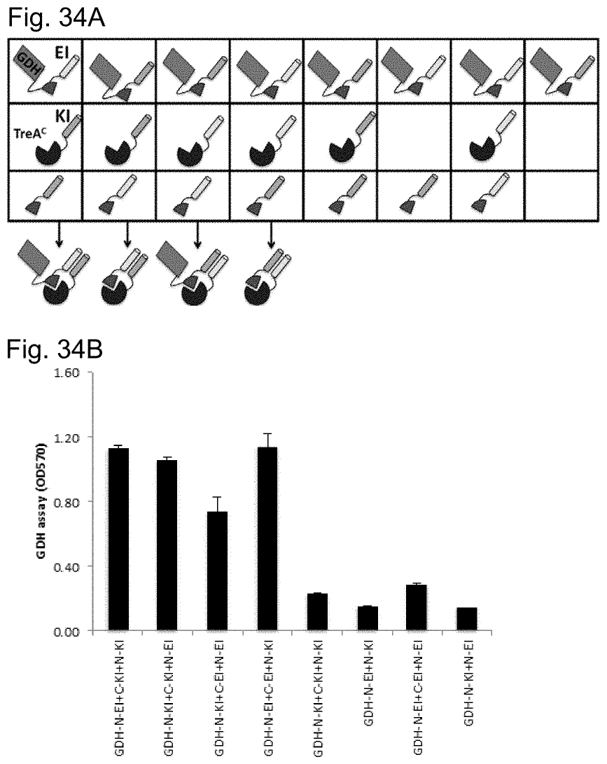

Complementation of the TreA fragments fused to heterodimerizing peptides EI and KI where GDH was fused to one of the complementing interaction partners (SEQ ID NOs: 71, 72), was compared with combinations where GDH was fused to TreA^{N} that did not engage in complementation (FIG. 34A). Care was taken that the stoichiometry of the reaction was identical in all the samples. First, it was shown that GDH can be used to measure the activity of complemented split TreA with specific GDH colorimetric reagents. It was also shown that the fusion of GDH did not interfere with the activity of either GDH or the complemented split TreA (FIG. 34B).

It is to be noted that the heterodimerizing peptides in the above disclosed fusions with TreA fragments and GDH, may be changed out for other analyte sensing domains such as pG, pA or pL (SEQ ID NOs: 73, 74, 75) to detect immunoglobulins. In fact, all analyte sensors could be incorporated into the GDH assays disclosed herein.

### Example 5

Disclosed in this example are that may be used to precisely detect and quantify Ca²⁺ ions present in blood samples. The split TreA calcium sensors comprise calmodulin-complexing elements fused to split TreA^{N} fragments and specific peptide-complexing elements fused to split TreA^{C} fragments. In the presence of Ca²⁺ ions, the calmodulin-complexing elements will bind to the peptide-complexing elements to thereby bind and restore TreA activity whereby supply of a trehalose substrate will result in glucose production that can be detected and measured with colorimetric assays or alternatively, with glucometers.

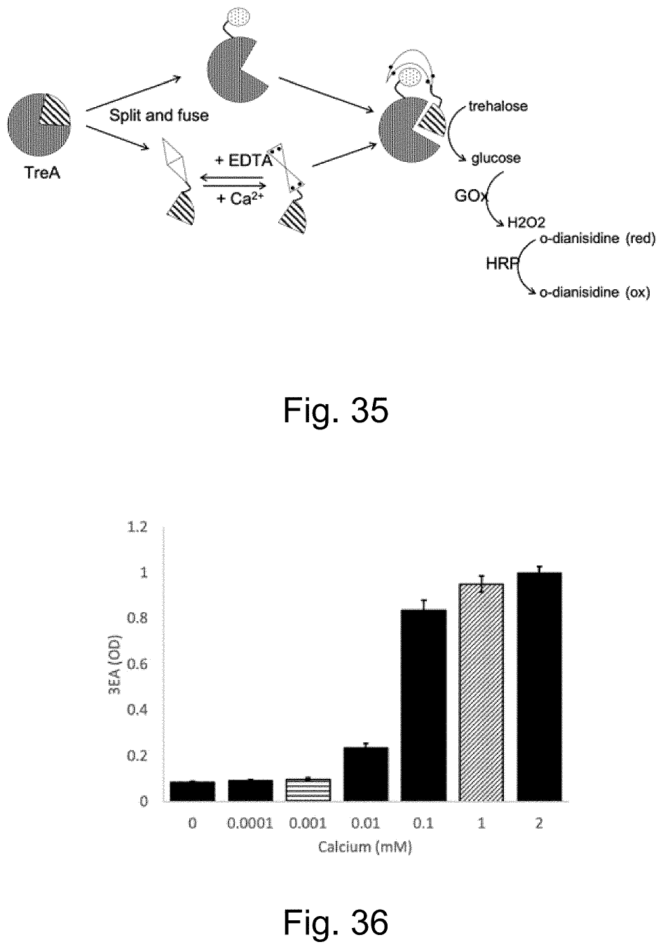

A schematic representation an example split TreA calcium sensor is illustrated in FIG. 35. The original TreA is shown with the N-terminal fragment in diagonal stripes and the C-terminal fragments in the vertical narrow stripes. After splitting, the TreA^{N} fragments are fused with Aspergillus nomius calmodulin protein (illustrated in FIG. 35 as two triangles extending outward from their conjoined bases), while the TreA^{C} fragments are fused with the M13-like peptide (illustrated in FIG. 36 as a sphere with a dotted centre). Binding of Ca²⁺ ions (illustrated in FIG. 36 as ●) to the calmodulin moieties will induce a conformational change (illustrated as two triangles conjoined at their apexes), thereby allowing the conformationally changed calmodulin moieties fused to the TreA^{N} moieties to bind to the M13-like peptide moieties fused to the TreA moieties. The interaction between TreA^{N}-calmodulin and the M13-TreA^{C} fusion proteins results in complementation of the split trehalase and restoration of its activity whereby a supply of trehalose substrate will result in its hydrolysis into glucose. Glucose production by the restored TreA activity can be measured by a colorimetric enzymatic reaction (3EA) using glucose oxidase (GOx) and horse radish peroxidase (HRP) and the substrate o-dianisidine.

It is to be noted that an element of calcium detection and monitoring assays in blood samples using the split TreA calcium sensor disclosed herein, is addition of the calcium-chelator, EDTA, to the reaction mixtures. Those skilled in this art may consider the addition of EDTA to said reaction mixtures to be counterintuitive because of EDTA's strong chelating properties. However, it was discovered that the chelating strength of EDTA is significantly reduced at neutral pHs and that addition of EDTA to the reaction mixtures at about neutral pHs provides proportional detection of Ca²⁺ ions in blood sample. It is to be noted that use of the EDTA in the present assays, strips all of the Ca²⁺ ions that bind to the proteins and/or anions in the sample, thereby providing precise measurement of total serum calcium (tCa) instead of only ionic calcium (iCa).

A TreA enzyme was split and the TreA^{N} fragment was fused with a partial coding sequence of calmodulin from Aspergillus nomius [NRRL 13137] (143 AA, residues: 28-170; XP_015401808) named N-Calm and cloned in the pETDuet expression vector to produce a sequence identified as SEQ ID NO. 80 and named TreA^{N}-Calm. The coding sequence of the corresponding M13-like peptide (RRTLHKAIDTVRAINKLREG; SEQ ID NO 81) was cloned N-terminally to the TreA^{C} fragment and named M13-TreA^{C} (SEQ ID NO: 82).

Pure Ca standards, serum and plasma samples were tested with the Ca split trehalase assay (referred to herein as “CalTreAx”), consisting of M13-TreA^{C} and TreA^{N}-Calm. Glucose measurements were determined with a GOx-HRP colorimetric assay (3EA) for all samples, consisting of glucose oxidase from Aspergillus niger, horseradish peroxidase, and O-dianisidine in sodium maleate buffer (50 mmol/L, pH 6). The results are shown in FIG. 36 wherein the bars show the concentrations of calcium ions added to the split TreA calcium sensor in log steps with CalTreAx reagents, after which the bound calcium ions were stripped by EDTA, after which, the EDTA was removed by dialysis. The threshold of detection (horizontal stripes) is given by significant difference with the 0 mM calcium samples. Saturation point (diagonal stripes) is the concentration at which the highest activity is reached. The experiment was run in triplicate.

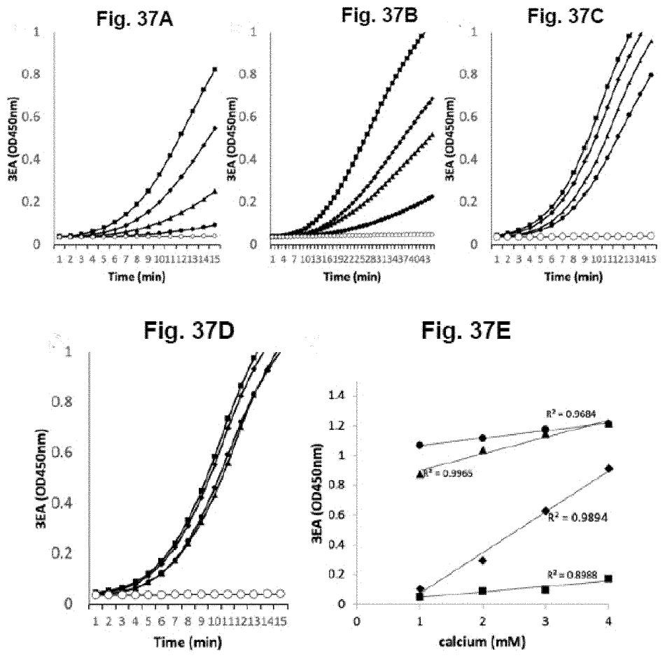

FIG. 37 is a series of charts showing the sensitivity of the CalTreAx assay for detection and quantification Ca²⁺ ions in serum samples in the presence of different concentrations of EDTA added to the reagents. FIG. 37A shows glucose production measured by the 3EA enzymatic reaction using GOx, HRP, and the substrate o-dianisidine in reaction mixtures with 4 (▪), 3 (♦), 2 (▴), 1 (●) and 0 (◯) mM of calcium in the presence of 25 mM EDTA. FIG. 37B shows glucose production measured by the 3EA enzymatic reaction using GOx, HRP, and the substrate o-dianisidine in reaction mixtures with 4 (▪), 3 (♦), 2 (▴), 1 (●) and 0 (◯) mM of calcium in the presence of 10 mM EDTA. FIG. 37C shows glucose production measured by the 3EA enzymatic reaction using GOx, HRP, and the substrate o-dianisidine in reaction mixtures with 4 (▪), 3 (♦), 2 (▴), 1 (●) and 0 (◯) mM of calcium in the presence of 2.5 mM EDTA. FIG. 37D shows glucose production measured by the 3EA enzymatic reaction using GOx, HRP, and the substrate o-dianisidine in reaction mixtures with 4 (▪), 3 (♦), 2 (▴), 1 (●) and 0 (◯) mM of calcium in the presence of 1 mM EDTA. FIG. 37E shows Pearson correlation plots between optical density measurements a 600 nm from 3EA reactions with the five Ca²⁺ ion concentrations for 25 (▪), 10 (♦), 2.5 (▴), 1 (●) mM of EDTA.

### Example 6

Disclosed in this example are split TreA calcium sensors that may be used to precisely detect and quantify bovine lactoferrin in milk samples.

It is known that lactoferrin concentrations increase during intramammary infections, in both clinical and subclinial cases (Harmon, R. J., et al., 1976, Changes in lactoferrin, immunoglobulin G, bovine serum albumin, and alpha-lactalbumin during acute experimental and natural coliform mastitis in cows. Infect. Immun. 13(2):533-542; Hagiwara, S., et al., 2003, Lactoferrin concentrations in milk from normal and subclinical mastitic cows. J. Vet. Med. Sci. 2003; 65(3):319-323; Hyvönen, P., et al., 2010, Concentrations of bovine lactoferrin and citrate in milk during experimental endotoxin mastitis in early-versus late-lactating dairy cows. J. Dairy Res. 77(4):474-480; Chaneton, L., et al., 2008, Relationship between milk lactoferrin and etiological agent in the mastitic bovine mammary gland. J. Dairy Sci. 91(5):1865-1873; Komine, K. et al., 2005, Small molecule lactoferrin with an inflammatory effect but no apparent antibacterial activity in mastitic mammary gland secretion. J Vet Med Sci. 2005; 67(7):667-677). Lactoferrin concentrations in chronic mastitis cases may be up to 7-fold higher than in healthy cows and may be 13-fold higher in acute mastitis cases during lactation. Mean milk lactoferrin concentrations for healthy cows in mid-lactation are in a range of about 0.01 to about 0.35 mg/ml, whereas in clinical and subclinical mastitis cases, lactoferrin increases significantly and may be as high as 3.6 mg/ml. Moreover, in persistent bacterial intramammary infections, milk lactoferrin concentrations are substantially increased. Also, there is a robust correlation between pathogen numbers in milk and lactoferrin concentrations, stronger in fact than between pathogen numbers and somatic cell counts (Chaneton, L., et al., 2008, J. Dairy Sci. 91(5):1865-1873; Hisaeda, K., 2016, Change in viable bacterial count during preservation of milk derived from dairy cows with subclinical mastitis and its relationship with antimicrobial components in milk. J. Vet. Med. Sci. 2016; 78(8):1245-50). Lactoferrin correlates with somatic cell counts and is associated with subsequent somatic cell counts increases in cows (Isobe, N., et al., 2013, Lingual antimicrobial peptide and lactoferrin concentrations and lactoperoxidase activity in bovine colostrum are associated with subsequent somatic cell count. Anim. Sci. J. 2013; 84(11):751-756).

It is known that calmodulin changes conformation in presence of calcium and binds to M13-like peptides derived from various proteins, including the Ca2+/calmodulin-dependent protein kinase II. The same could be achieved with peptides derived from myosin light chain kinase, calcineurin, spectrin, and olfactory cyclic-nucleotide gated channel.

It is known that human lactoferrin binds to human calmodulin and that cow calmodulin is identical to human calmodulin. However, bovine lactoferrin is not identical to human lactoferrin.

It was discovered that split TreA calcium sensors may also be used to detect and quantify the presence of bovine lactoferrin in milk samples.

A TreA enzyme was split and the TreA^{N} fragment was fused with a partial coding sequence of bovine calmodulin, named bCalm and cloned in the pETDuet expression vector to produced sequence identified as SEQ ID NO. 83 and named TreA^{N}-bCalm. The coding sequence of the corresponding CaMKII peptide (SEQ ID NO 84) was cloned N-terminally to the TreA^{C} fragment and named CaMKIIp-TreA^{C} (SEQ ID NO: 85).

In a first study, the presence of bovine lactoferrin in 90 milk samples was determined with an ELISA test and a full range of somatic cell counts, and determined that the bovine lactoferrin concentration averaged 1 mg/ml (with low Quartile below 0.6 mg/ml and high Quartile above 1.5 mg/ml).

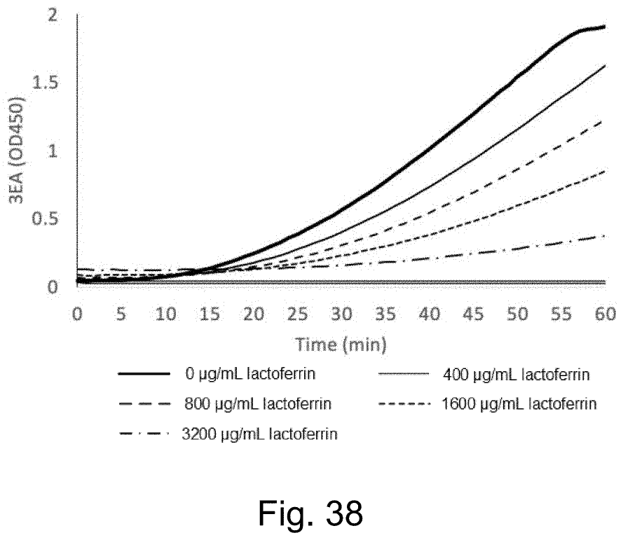

It was demonstrated that in the presence of Ca²⁺ ions, a Ca split trehalase assay consisting of TreA^{N}-bCalm (SEQ ID NO:83) and CaMKIIp-TreA^{C} (SEQ ID NO: 85) bind and enable complementation of the trehalase enzyme fragments and activation of the trehalase activity. It was demonstrated that increasing concentrations of bovine lactoferrin (0, 400, 800, 1600, 3200 μg/mL) proportionally hinder the binding of calmodulin to the CaMKII peptide in the presence of 4 mM CaCl₂) and 10 mM EDTA, thereby reducing the complementation of the TreA fragments, resulting in lower glucose production (FIG. 38). Furthermore, it was demonstrated that the reduction in the complementation of the TreA fragments is specific to the presence of bovine lactoferrin whereas repeating the assay with same concentrations of bovine serum albumin did not have any inhibitory effects.

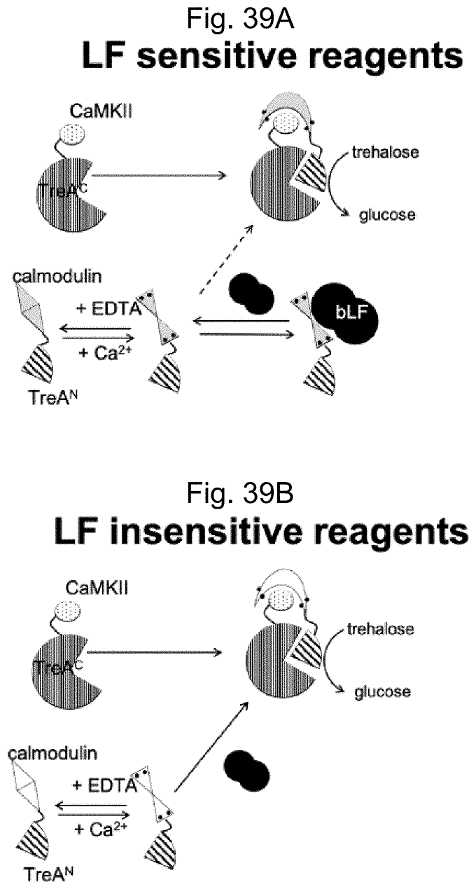

Accordingly, a fungal calmodulin fused to the N-terminal fragment of TreA (TreA^{N}_Calm) (SEQ ID NO: 80) will bind to the MaMKII peptide fused to C-terminal fragment of TreA (SEQ ID NO: 85) and complements the TreA fragments, but that this complementation is not inhibited by bovine lactoferrin, because it does not have affinity for this calmodulin (FIGS. 39A, 39B). As illustrated in FIGS. 39A, 39B, bovine calmodulin (shown in grey) has an affinity for bovine lactoferrin (bLF) (black). Calcium binds to an open structure of calmodulin, which in turn gains affinity for the CaMKII peptide. Fungal calmodulin (shown in white) does not have any affinity for bovine lactoferrin. The presence of bLF hinders the complex formation of CaMKIIp-TreA^{C} and TreA^{N}-calm and thereby reduces resulting trehalase activity as evidenced by glucose production in presence of calcium (●). The availability of both sensitive and insensitive reagents enables comparisons of the effects of lactoferrin to a maximum trehalase activity, thereby providing a read-out for the amount of lactoferrin present in a sample.

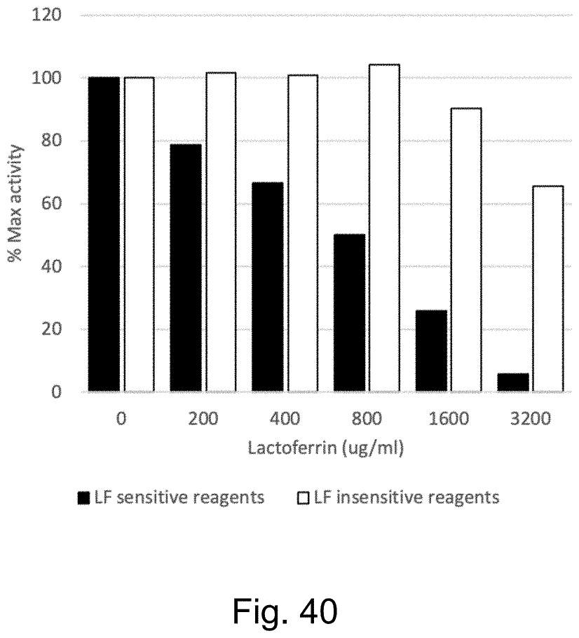

A study was done wherein reactions were performed with (i) lactoferrin-sensitive reagents TreA^{N}-bCalm (SEQ ID NO: 83) and Ca MKIIp-TreA^{C} (SEQ ID NO: 85), and (ii) lactoferrin-insensitive reagents TreA^{N}-Calm (SEQ ID NO: 80) and CaMKIIp-TreA (SEQ ID NO: 85) in the presence of 4 mM CaCl₂) and 10 mM EDTA with 0, 200, 400, 800, 1600, and 3200 μg/mL of bovine lactoferrin added to the reaction mixtures. “0” lactoferrin was set as 100% activity. Glucose measurements in the different reaction mixtures were determined with a GOx-HRP colorimetric assay (3EA) for all samples, consisting of GOx from Aspergillus niger, HRP, and O-dianisidine substrate in sodium maleate buffer (50 mmol/L, pH 6). The data in FIG. 40 show that glucose production was increasing hindered in the assays with the lactoferrin-sensitive reagents TreA^{N}-bCalm (SEQ ID NO: 83) and Ca MKIIp-TreA^{C} (SEQ ID NO: 85) as lactoferrin concentrations increased (black bars) while glucose production in assays with the lactoferrin-insensitive reagents TreA^{N}-Calm (SEQ ID NO: 80) and CaMKIIp-TreA^{C} (SEQ ID NO: 85) was not affected (white bars).

In summary, it was discovered that the split TreA calcium sensors disclosed herein may also be used to detect and quantify the presence of bovine lactoferrin in milk samples, for example, by contacting a first subsample of a milk sample with lactoferrin-insensitive reagents TreA^{N}-Calm (SEQ ID NO: 80) and CaMKIIp-TreA^{C} (SEQ ID NO: 85) to produce a first detectable glucose production resulting from the fusion of the TreA^{N}-Calm and CaMKIIp-TreA^{C} reagents by Ca²⁺ ions present in the milk sample. A second subsample of the milk sample may be contacted with lactoferrin-sensitive reagents TreA^{N}-bCalm (SEQ ID NO: 83) and CaMKIIp-TreA^{C} (SEQ ID NO: 85) to produce a second detectable glucose production resulting from the fusion of the TreA^{N}-bCalm and CaMKIIp-TreA^{C} reagents by Ca²⁺ ions present in the milk sample. The presence of lactoferrin in the milk sample will result in lactoferrin moieties fusing to the TreA^{N}-bCalm reagent thereby reducing the number of TreA^{N}_bCalm moieties that are available for fusion with the CaMKIIp-TreA^{C} reagent moieties by the Ca²⁺ ions present in the milk sample. A positive test result for the presence of lactoferrin in a milk sample occurs when the second detected glucose production by the lactoferrin-sensitive reagents TreA^{N}-bCalm (SEQ ID NO: 83) and CaMKIIp-TreA^{C} (SEQ ID NO: 85) is lower than the first detected glucose production by the lactoferrin-insensitive reagents TreA^{N}-Calm (SEQ ID NO: 83) and CaMKIIp-TreA^{C} (SEQ ID NO: 85). The magnitude of difference in glucose production by the contacting subsamples of a milk sample with the (i) lactoferrin-insensitive reagents, and the lactoferrin-insensitive reagents of the split TreA calcium sensors disclosed herein, may be used as an indicator of severity of a bovine mastitis occurrence.

SEQUENCES:
SEQ ID NO: 1: Forward primer for preparation of the Tn5 (aph) type II
(kanamycin resistance) replacement for the entire TreA gene
TATGGACAGCAAGCGAACCG
SEQ ID NO: 2: Reverse primer for preparation of the Tn5 (aph) type II
(kanamycin resistance) replacement for the entire TreA gene
TCAGAAGAACTCGTCAAGAAG
SEQ ID NOs: 3-13: Primers shown in Table 1
SEQ ID NO: 14: Protein G
TYKLILNGKTLKGETTTEAVDAATAEKVFKQYANDNGVDGEWTYDAATKTFTVTE
SEQ ID NO: 15: Protein A
TADNKFNKEQQNAFYEILHLPNLNEEQRNGFIQSLKDDPSQSANLLAEAKKLNDAQAPKA
SEQ ID NO: 16: Protein L
AMEEVTIKANLIFANGSTQTAEFKGTFEKATSEAYAYADTLKKDNGEWTVDVADKGYTLNIKFAG
SEQ ID NO: 17: HIS-TreA: N-terminally HIS-tagged TreA (w/o signal peptide)
MAHHHHHHEETPVTPQPPDILLGPLFNDVQNAKLFPDQKTFADAVPNSDPLMILADYRMQQNQSGFDLR
HFVNVNFTLPKEGEKYVPPEGQSLREHIDGLWPVLTRSTENTEKWDSLLPLPEPYVVPGGRFREVYYWD
SYFTMLGLAESGHWDKVADMVANFAHEIDTYGHIPNGNRSYYLSRSQPPFFALMVELLAQHEGDAALKQ
YLPQMQKEYAYWMDGVENLQAGQQEKRVVKLQDGTLLNRYWDDRDTPRPESWVEDIATAKSNPNRPA
TEIYRDLRSAAASGWDFSSRWMDNPQQLNTLRTTSIVPVDLNSLMFKMEKILARASKAAGDNAMANQYE
TLANARQKGIEKYLWNDQQGWYADYDLKSHKVRNQLTAAALFPLYVNAAAKDRANKMATATKTHLLQPG
GLNTTSVKSGQQWDAPNGWAPLQWVATEGLQNYGQKEVAMDISWHFLTNVQHTYDREKKLVEKYDVS
TTGTGGGGGEYPLQDGFGWTNGVTLKMLDLICPKEQPCDNVPATRPTVKSATTQPSTKEAQPTP
SEQ ID NO: 18: TreA-HIS: C-terminally HIS-tagged TreA (w/o signal peptide)
MEETPVTPQPPDILLGPLFNDVQNAKLFPDQKTFADAVPNSDPLMILADYRMQQNQSGFDLRHFVNVNF
TLPKEGEKYVPPEGQSLREHIDGLWPVLTRSTENTEKWDSLLPLPEPYVVPGGRFREVYYWDSYFTMLG
LAESGHWDKVADMVANFAHEIDTYGHIPNGNRSYYLSRSQPPFFALMVELLAQHEGDAALKQYLPQMQK
EYAYWMDGVENLQAGQQEKRVVKLQDGTLLNRYWDDRDTPRPESWVEDIATAKSNPNRPATEIYRDLR
SAAASGWDFSSRWMDNPQQLNTLRTTSIVPVDLNSLMFKMEKILARASKAAGDNAMANQYETLANARQ
KGIEKYLWNDQQGWYADYDLKSHKVRNQLTAAALFPLYVNAAAKDRANKMATATKTHLLQPGGLNTTSV
KSGQQWDAPNGWAPLQWVATEGLQNYGQKEVAMDISWHFLTNVQHTYDREKKLVEKYDVSTTGTGG
GGGEYPLQDGFGWTNGVTLKMLDLICPKEQPCDNVPATRPTVKSATTQPSTKEAQPTPHHHHHH
SEQ ID NO: 19: HIS-TreA-HIS: N- and C- terminally HIS-tagged TreA (w/o
signal peptide)
MAHHHHHHEETPVTPQPPDILLGPLFNDVQNAKLFPDQKTFADAVPNSDPLMILADYRMQQNQSGFDLR
HFVNVNFTLPKEGEKYVPPEGQSLREHIDGLWPVLTRSTENTEKWDSLLPLPEPYVVPGGRFREVYYWD
SYFTMLGLAESGHWDKVADMVANFAHEIDTYGHIPNGNRSYYLSRSQPPFFALMVELLAQHEGDAALKQ
YLPQMQKEYAYWMDGVENLQAGQQEKRVVKLQDGTLLNRYWDDRDTPRPESWVEDIATAKSNPNRPA
TEIYRDLRSAAASGWDFSSRWMDNPQQLNTLRTTSIVPVDLNSLMFKMEKILARASKAAGDNAMANQYE
TLANARQKGIEKYLWNDQQGWYADYDLKSHKVRNQLTAAALFPLYVNAAAKDRANKMATATKTHLLQPG
GLNTTSVKSGQQWDAPNGWAPLQWVATEGLQNYGQKEVAMDISWHFLTNVQHTYDREKKLVEKYDVS
TTGTGGGGGEYPLQDGFGWTNGVTLKMLDLICPKEQPCDNVPATRPTVKSATTQPSTKEAQPTPHHHH
HH
SEQ ID NO: 20: HIS-TreAN: N-terminally HIS-tagged N-terminal fragment
of TreA (w/o signal peptide)
MAHHHHHHEETPVTPQPPDILLGPLFNDVQNAKLFPDQKTFADAVPNSDPLMILADYRMQQNQSGFDLR
HFVNVNFTLPKEGEKYVPGS
SEQ ID NO: 21: TreAN-HIS: C-terminally HIS-tagged N-terminal fragment
of TreA (w/o signal peptide)
MEETPVTPQPPDILLGPLFNDVQNAKLFPDQKTFADAVPNSDPLMILADYRMQQNQSGFDLRHFVNVNF
TLPKEGEKYVPHHHHHH
SEQ ID NO: 22: HIS-TreAC: N-terminally HIS-tagged C-terminal fragment
of TreA (w/o signal peptide)
MAHHHHHHNFTLPKEGEKYVPPEGQSLREHIDGLWPVLTRSTENTEKWDSLLPLPEPYVVPGGRFREVY
YWDSYFTMLGLAESGHWDKVADMVANFAHEIDTYGHIPNGNRSYYLSRSQPPFFALMVELLAQHEGDA
ALKQYLPQMQKEYAYWMDGVENLQAGQQEKRVVKLQDGTLLNRYWDDRDTPRPESWVEDIATAKSNP
NRPATEIYRDLRSAAASGWDFSSRWMDNPQQLNTLRTTSIVPVDLNSLMFKMEKILARASKAAGDNAMA
NQYETLANARQKGIEKYLWNDQQGWYADYDLKSHKVRNQLTAAALFPLYVNAAAKDRANKMATATKTH
LLQPGGLNTTSVKSGQQWDAPNGWAPLQWVATEGLQNYGQKEVAMDISWHFLTNVQHTYDREKKLVE
KYDVSTTGTGGGGGEYPLQDGFGWTNGVTLKMLDLICPKEQPCDNVPATRPTVKSATTQPSTKEAQPT
P
SEQ ID NO: 23: TreAC-HIS: C-terminally HIS-tagged C-terminal fragment
of TreA (w/o signal peptide)
MAVNFTLPKEGEKYVPPEGQSLREHIDGLWPVLTRSTENTEKWDSLLPLPEPYVVPGGRFREVYYWDSY
FTMLGLAESGHWDKVADMVANFAHEIDTYGHIPNGNRSYYLSRSQPPFFALMVELLAQHEGDAALKQYL
PQMQKEYAYWMDGVENLQAGQQEKRVVKLQDGTLLNRYWDDRDTPRPESWVEDIATAKSNPNRPATE
IYRDLRSAAASGWDFSSRWMDNPQQLNTLRTTSIVPVDLNSLMFKMEKILARASKAAGDNAMANQYETL
ANARQKGIEKYLWNDQQGWYADYDLKSHKVRNQLTAAALFPLYVNAAAKDRANKMATATKTHLLQPGG
LNTTSVKSGQQWDAPNGWAPLQWVATEGLQNYGQKEVAMDISWHFLTNVQHTYDREKKLVEKYDVST
TGTGGGGGEYPLQDGFGWTNGVTLKMLDLICPKEQPCDNVPATRPTVKSATTQPSTKEAQPTPHHHHH
H
SEQ ID NO: 24: HA-TreAN-HIS: N-terminally HA-tagged, C-terminally
HIS-tagged N-terminal fragment of TreA (w/o signal peptide)
MAYPYDVPDYAVDEETPVTPQPPDILLGPLFNDVQNAKLFPDQKTFADAVPNSDPLMILADYRMQQNQS
GFDLRHFVNVNFTLPKEGEKYVPHHHHHH
SEQ ID NO: 25: HIS-TreAN-HA: N-terminally HIS-tagged, C-terminally
HA-tagged N-terminal fragment of TreA (w/o signal peptide)
MAHHHHHHEETPVTPQPPDILLGPLFNDVQNAKLFPDQKTFADAVPNSDPLMILADYRMQQNQSGFDLR
HFVNVNFTLPKEGEKYVPGSYPYDVPDYA
SEQ ID NO: 26: HA-TreAC-HIS: N-terminally HA-tagged, C-terminally
HIS-tagged C-terminal fragment of TreA
MAYPYDVPDYAVDNFTLPKEGEKYVPPEGQSLREHIDGLWPVLTRSTENTEKWDSLLPLPEPYVVPGGR
FREVYYWDSYFTMLGLAESGHWDKVADMVANFAHEIDTYGHIPNGNRSYYLSRSQPPFFALMVELLAQH
EGDAALKQYLPQMQKEYAYWMDGVENLQAGQQEKRVVKLQDGTLLNRYWDDRDTPRPESWVEDIATA
KSNPNRPATEIYRDLRSAAASGWDFSSRWMDNPQQLNTLRTTSIVPVDLNSLMFKMEKILARASKAAGD
NAMANQYETLANARQKGIEKYLWNDQQGWYADYDLKSHKVRNQLTAAALFPLYVNAAAKDRANKMATA
TKTHLLQPGGLNTTSVKSGQQWDAPNGWAPLQWVATEGLQNYGQKEVAMDISWHFLTNVQHTYDREK
KLVEKYDVSTTGTGGG
GGEYPLQDGFGWTNGVTLKMLDLICPKEQPCDNVPATRPTVKSATTQPSTKEAQPTPHHHHHH
SEQ ID NO: 27: HIS-TreAC-HA: N-terminally HIS-tagged, C-terminally
HA-tagged C-terminal fragment of TreA
MAHHHHHHNFTLPKEGEKYVPPEGQSLREHIDGLWPVLTRSTENTEKWDSLLPLPEPYVVPGGRFREVY
YWDSYFTMLGLAESGHWDKVADMVANFAHEIDTYGHIPNGNRSYYLSRSQPPFFALMVELLAQHEGDA
ALKQYLPQMQKEYAYWMDGVENLQAGQQEKRVVKLQDGTLLNRYWDDRDTPRPESWVEDIATAKSNP
NRPATEIYRDLRSAAASGWDFSSRWMDNPQQLNTLRTTSIVPVDLNSLMFKMEKILARASKAAGDNAMA
NQYETLANARQKGIEKYLWNDQQGWYADYDLKSHKVRNQLTAAALFPLYVNAAAKDRANKMATATKTH
LLQPGGLNTTSVKSGQQWDAPNGWAPLQWVATEGLQNYGQKEVAMDISWHFLTNVQHTYDREKKLVE
KYDVSTTGTGGGGGEYPLQDGFGWTNGVTLKMLDLICPKEQPCDNVPATRPTVKSATTQPSTKEAQPT
PGSYPYDVPDYA
SEQ ID NO: 28: HIS-TreAN-ProtG: N-terminally HIS-tagged N-terminal
fragment of TreA with C-terminal fusion with immunoglobulin binding
Domain B1 of protein G of Streptococcus
MAHHHHHHEETPVTPQPPDILLGPLFNDVQNAKLFPDQKTFADAVPNSDPLMILADYRMQQNQSGFDLR
HFVNVNFTLPKEGEKYVPGSVPGS
TYKLILNGKTLKGETTTEAVDAATAEKVFKQYANDNGVDGEWYDAATKTFTVTE
SEQ ID NO: 29: HIS-TreAC-ProtG: N-terminally HIS-tagged N-terminal
fragment of TreA with C-terminal fusion with immunoglobulin binding
Domain B1 of protein G of Streptococcus
MAHHHHHHNFTLPKEGEKYVPPEGQSLREHIDGLWPVLTRSTENTEKWDSLLPLPEPYVVPGGRFREVY
YWDSYFTMLGLAESGHWDKVADMVANFAHEIDTYGHIPNGNRSYYLSRSQPPFFALMVELLAQHEGDA
ALKQYLPQMQKEYAYWMDGVENLQAGQQEKRVVKLQDGTLLNRYWDDRDTPRPESWVEDIATAKSNP
NRPATEIYRDLRSAAASGWDFSSRWMDNPQQLNTLRTTSIVPVDLNSLMFKMEKILARASKAAGDNAMA
NQYETLANARQKGIEKYLWNDQQGWYADYDLKSHKVRNQLTAAALFPLYVNAAAKDRANKMATATKTH
LLQPGGLNTTSVKSGQQWDAPNGWAPLQWVATEGLQNYGQKEVAMDISWHFLTNVQHTYDREKKLVE
KYDVSTTGTGGGGGEYPLQDGFGWTNGVTLKMLDLICPKEQPCDNVPATRPTVKSATTQPSTKEAQPT
PGSTYKLILNGKTLKGETTTEAVDAATAEKVFKQYANDNGVDGEWYDAATKTFTVTE
SEQ ID NO: 30: HIVP24-TreAN-HIS: HIV capside protein P24 fused
N-terminally to the N-terminal fragment of TreA (w/o signal peptide),
C-terminal HIS-tag
MEPIVQNIQGQMVHQAISPRTLNAWVKVVEEKAFSPEVIPMFSALSEGATPQDLNTMLNTVGGHQAAMQ
MLKETINEEAAEWDRVHPVHAGPIAPGQMREPRGSDIAGTTSTLQEQIGWMTNNPPIPVGEIYKRWIILGL
NKIVRMYSPTSILDIRQGPKEPFRDYVDRFYKTLRAEQASQEVKNAMTETLLVQNANPDCKTILKALGPAA
TLEEMMTACQGVGGPGHKARVLVDEETPVTPQPPDILLGPLFNDVQNAKLFPDQKTFADAVPNSDPLMIL
ADYRMQQNQSGFDLRHFVNVNFTLPKEGEKYVPHHHHHH
SEQ ID NO: 31: HIVP24-TreAC: HIV capside protein P24 fused N-terminally
to the C-terminal fragment of TreA, with C-terminal HIS-tag
MEPIVQNIQGQMVHQAISPRTLNAWVKVVEEKAFSPEVIPMFSALSEGATPQDLNTMLNTVGGHQAAMQ
MLKETINEEAAEWDRVHPVHAGPIAPGQMREPRGSDIAGTTSTLQEQIGWMTNNPPIPVGEIYKRWIILGL
NKIVRMYSPTSILDIRQGPKEPFRDYVDRFYKTLRAEQASQEVKNAMTETLLVQNANPDCKTILKALGPAA
TLEEMMTACQGVGGPGHKARVLVDNFTLPKEGEKYVPPEGQSLREHIDGLWPVLTRSTENTEKWDSLL
PLPEPYVVPGGRFREVYYWDSYFTMLGLAESGHWDKVADMVANFAHEIDTYGHIPNGNRSYYLSRSQP
PFFALMVELLAQHEGDAALKQYLPQMQKEYAYWMDGVENLQAGQQEKRVVKLQDGTLLNRYWDDRDT
PRPESWVEDIATAKSNPNRPATEIYRDLRSAAASGWDFSSRWMDNPQQLNTLRTTSIVPVDLNSLMFKM
EKILARASKAAGDNAMANQYETLANARQKGIEKYLWNDQQGWYADYDLKSHKVRNQLTAAALFPLYVNA
AAKDRANKMATATKTHLLQPGGLNTTSVKSGQQWDAPNGWAPLQWVATEGLQNYGQKEVAMDISWHF
LTNVQHTYDREKKLVEKYDVSTTGTGGGGGEYPLQDGFGWTNGVTLKMLDLICPKEQPCDNVPATRPT
VKSATTQPSTKEAQPTPHHHHHH
SEQ ID NO 32: TreAN-pA: N-terminally HIS-tagged N-terminus of TreA
(w/o signal peptide) C-terminally fused with the immunoglobulin binding
domain of protein A
MAHHHHHHEETPVTPQPPDILLGPLFNDVQNAKLFPDQKTFADAVPNSDPLMILADYRMQQNQSGFDLR
HFVNVNFTLPKEGEKYVPGSAMEEVTIKANLIFANGSTQTAEFKGTFEKATSEAYAYADTLKKDNGEWTV
DVADKGYTLNIKFAG
SEQ ID NO 33: TreAC-pA: N-terminally HIS-tagged C-terminus of TreA
(w/o signal peptide) C-terminally fused with the immunoglobulin binding
domain of protein A
MAHHHHHHNFTLPKEGEKYVPPEGQSLREHIDGLWPVLTRSTENTEKWDSLLPLPEPYVVPGGRFREVY
YWDSYFTMLGLAESGHWDKVADMVANFAHEIDTYGHIPNGNRSYYLSRSQPPFFALMVELLAQHEGDA
ALKQYLPQMQKEYAYWMDGVENLQAGQQEKRVVKLQDGTLLNRYWDDRDTPRPESWVEDIATAKSNP
NRPATEIYRDLRSAAASGWDFSSRWMDNPQQLNTLRTTSIVPVDLNSLMFKMEKILARASKAAGDNAMA
NQYETLANARQKGVEKYLWNDQQGWYADYDLKSHKVRNQLTAAALFPLYVNAAAKDRANKMATATKTH
LLQPGGLNTTSVKSGQQWDAPNGWAPLQWVATEGLQNYGQKEVAMDISWHFLTNGQHTYDREKKLVE
KYDVSTTGTGGGGGEYPLQDGFGWTNGVTLKMLDLICPKEQPCDNVPATRPTVKSATTQPSTKEAQPT
PGSTADNKFNKEQQNAFYEILHLPNLNEEQRNGFIQSLKDDPSQSANLLAEAKKLNDAQAPKA
SEQ ID NO 34: TreAN-pL: N-terminally HIS-tagged N-terminus of TreA
(w/o signal peptide) C-terminally fused with the immunoglobulin binding
domain of protein L
MAHHHHHHEETPVTPQPPDILLGPLFNDVQNAKLFPDQKTFADAVPNSDPLMILADYRMQQNQSGFDLR
HFVNVNFTLPKEGEKYVPGSTADNKFNKEQQNAFYEILHLPNLNEEQRNGFIQSLKDDPSQSANLLAEAK
KLNDAQAPKA
SEQ ID NO 35: TreAC-pL: N-terminally HIS-tagged C-terminus of TreA
(w/o signal peptide) C-terminally fused with the immunoglobulin binding
domain of protein L
MAHHHHHHNFTLPKEGEKYVPPEGQSLREHIDGLWPVLTRSTENTEKWDSLLPLPEPYVVPGGRFREVY
YWDSYFTMLGLAESGHWDKVADMVANFAHEIDTYGHIPNGNRSYYLSRSQPPFFALMVELLAQHEGDA
ALKQYLPQMQKEYAYWMDGVENLQAGQQEKRVVKLQDGTLLNRYWDDRDTPRPESWVEDIATAKSNP
NRPATEIYRDLRSAAASGWDFSSRWMDNPQQLNTLRTTSIVPVDLNSLMFKMEKILARASKAAGDNAMA
NQYETLANARQKGVEKYLWNDQQGWYADYDLKSHKVRNQLTAAALFPLYVNAAAKDRANKMATATKTH
LLQPGGLNTTSVKSGQQWDAPNGWAPLQWVATEGLQNYGQKEVAMDISWHFLTNGQHTYDREKKLVE
KYDVSTTGTGGGGGEYPLQDGFGWTNGVTLKMLDLICPKEQPCDNVPATRPTVKSATTQPSTKEAQPT
PGSAMEEVTIKANLIFANGSTQTAEFKGTFEKATSEAYAYADTLKKDNGEWTVDVADKGYTLNIKFAG
SEQ ID NO: 36: Staphylococcus aureus binding peptide SA5-1
VPHNPGLISLQG
SEQ ID NO: 37: Mycobacterium avium subsp. paratuberculosis
binding peptide Mp3
NYVIHDVPRHPA
SEQ ID NO: 38: complementary coiled-coil peptide with leucine zipper
motif Ei
EIAALEKEIAALEKENAALEWEIAALEK
SEQ ID NO: 39: complementary coiled-coil peptide with leucine zipper
motif Ki
KIAALKEKIAALKEKNAALKWKIAALKE
SEQ ID NO: 40: TreAN-SA: N-terminally HIS-tagged N-terminal fragment
of TreA with C-terminal fusion with S. aureus binding peptide
aptamer, SA5-1
MAHHHHHHEETPVTPQPPDILLGPLFNDVQNAKLFPDQKTFADAVPNSDPLMILADYRMQQNQSGFDLR
HFVNVNFTLPKEGEKYVPGSVPHNPGLISLQG
SEQ ID NO: 41: SA-TreAN: C-terminally HIS-tagged N-terminal fragment
of TreA with N-terminal fusion with S. aureus binding peptide
aptamer, SA5-1
MVPHNPGLISLQGVDEETPVTPQPPDILLGPLFNDVQNAKLFPDQKTFADAVPNSDPLMILADYRMQQNQ
SGFDLRHFVNVNFTLPKEGEKYVPHHHHHH
SEQ ID NO: 42: TreAC-SA: N-terminally HIS-tagged C-terminal fragment
of TreA with C-terminal fusion with S. aureus binding peptide aptamer,
SA5-1
MAHHHHHHNFTLPKEGEKYVPPEGQSLREHIDGLWPVLTRSTENTEKWDSLLPLPEPYVVPGGRFREVY
YWDSYFTMLGLAESGHWDKVADMVANFAHEIDTYGHIPNGNRSYYLSRSQPPFFALMVELLAQHEGDA
ALKQYLPQMQKEYAYWMDGVENLQAGQQEKRVVKLQDGTLLNRYWDDRDTPRPESWVEDIATAKSNP
NRPATEIYRDLRSAAASGWDFSSRWMDNPQQLNTLRTTSIVPVDLNSLMFKMEKILARASKAAGDNAMA
NQYETLANARQKGIEKYLWNDQQGWYADYDLKSHKVRNQLTAAALFPLYVNAAAKDRANKMATATKTH
LLQPGGLNTTSVKSGQQWDAPNGWAPLQWVATEGLQNYGQKEVAMDISWHFLTNVQHTYDREKKLVE
KYDVSTTGTGGGGGEYPLQDGFGWTNGVTLKMLDLICPKEQPCDNVPATRPTVKSATTQPSTKEAQPT
PGSVPHNPGLISLQG
SEQ ID NO: 43: SA-TreAC: C-terminally HIS-tagged C-terminal fragment
of TreA with N-terminal fusion with S. aureus binding peptide aptamer,
SA5-1
MVPHNPGLISLQGVDNFTLPKEGEKYVPPEGQSLREHIDGLWPVLTRSTENTEKWDSLLPLPEPYVVPG
GRFREVYYWDSYFTMLGLAESGHWDKVADMVANFAHEIDTYGHIPNGNRSYYLSRSQPPFFALMVELLA
QHEGDAALKQYLPQMQKEYAYWMDGVENLQAGQQEKRVVKLQDGTLLNRYWDDRDTPRPESWVEDIA
TAKSNPNRPATEIYRDLRSAAASGWDFSSRWMDNPQQLNTLRTTSIVPVDLNSLMFKMEKILARASKAAG
DNAMANQYETLANARQKGIEKYLWNDQQGWYADYDLKSHKVRNQLTAAALFPLYVNAAAKDRANKMAT
ATKTHLLQPGGLNTTSVKSGQQWDAPNGWAPLQWVATEGLQNYGQKEVAMDISWHFLTNVQHTYDRE
KKLVEKYDVSTTGTGGGGGEYPLQDGFGWTNGVTLKMLDLICPKEQPCDNVPATRPTVKSATTQPSTKE
AQPTPHHHHHH
SEQ ID NO: 44: TreAN-Mp3: N-terminally HIS-tagged N-terminal fragment
of TreA with C-terminal fusion with M. avium subsp. paratuberculosis
binding peptide aptamer, Mp3
MAHHHHHHEETPVTPQPPDILLGPLFNDVQNAKLFPDQKTFADAVPNSDPLMILADYRMQQNQSGFDLR
HFVNVNFTLPKEGEKYVPGSNYVIHDVPRHPA
SEQ ID NO: 45: Mp3-TreAN: C-terminally HIS-tagged N-terminal fragment
of TreA with N-terminal fusion with M. avium subsp. paratuberculosis
binding peptide aptamer, Mp3
MGNYVIHDVPRHPAVDEETPVTPQPPDILLGPLFNDVQNAKLFPDQKTFADAVPNSDPLMILADYRMQQN
QSGFDLRHFVNVNFTLPKEGEKYVPHHHHHH
SEQ ID NO: 46: TreAC-Mp3: N-terminally HIS-tagged C-terminal fragment
of TreA with C-terminal fusion with M. avium subsp. paratuberculosis
binding peptide aptamer, Mp3
MAHHHHHHNFTLPKEGEKYVPPEGQSLREHIDGLWPVLTRSTENTEKWDSLLPLPEPYVVPGGRFREVY
YWDSYFTMLGLAESGHWDKVADMVANFAHEIDTYGHIPNGNRSYYLSRSQPPFFALMVELLAQHEGDA
ALKQYLPQMQKEYAYWMDGVENLQAGQQEKRVVKLQDGTLLNRYWDDRDTPRPESWVEDIATAKSNP
NRPATEIYRDLRSAAASGWDFSSRWMDNPQQLNTLRTTSIVPVDLNSLMFKMEKILARASKAAGDNAMA
NQYETLANARQKGIEKYLWNDQQGWYADYDLKSHKVRNQLTAAALFPLYVNAAAKDRANKMATATKTH
LLQPGGLNTTSVKSGQQWDAPNGWAPLQWVATEGLQNYGQKEVAMDISWHFLTNVQHTYDREKKLVE
KYDVSTTGTGGGGGEYPLQDGFGWTNGVTLKMLDLICPKEQPCDNVPATRPTVKSATTQPSTKEAQPT
PGSNYVIHDVPRHPA
SEQ ID NO: 47: Mp3-TreAC: C-terminally HIS-tagged C-terminal fragment
of TreA with N-terminal fusion with M. avium subsp. paratuberculosis
binding peptide aptamer, Mp3
MGNYVIHDVPRHPAVDNFTLPKEGEKYVPPEGQSLREHIDGLWPVLTRSTENTEKWDSLLPLPEPYVVP
GGRFREVYYWDSYFTMLGLAESGHWDKVADMVANFAHEIDTYGHIPNGNRSYYLSRSQPPFFALMVELL
AQHEGDAALKQYLPQMQKEYAYWMDGVENLQAGQQEKRVVKLQDGTLLNRYWDDRDTPRPESWVEDI
ATAKSNPNRPATEIYRDLRSAAASGWDFSSRWMDNPQQLNTLRTTSIVPVDLNSLMFKMEKILARASKAA
GDNAMANQYETLANARQKGIEKYLWNDQQGWYADYDLKSHKVRNQLTAAALFPLYVNAAAKDRANKMA
TATKTHLLQPGGLNTTSVKSGQQWDAPNGWAPLQWVATEGLQNYGQKEVAMDISWHFLTNVQHTYDR
EKKLVEKYDVSTTGTGGGGGEYPLQDGFGWTNGVTLKMLDLICPKEQPCDNVPATRPTVKSATTQPSTK
EAQPTPHHHHHH
SEQ ID NO: 48: scFvLm-TreA-HIS: N-terminally HIS-tagged TreA C-terminally
fused with Listeria monocytogenes ActA protein specific single chain
fragment variable
MGKYLLPTAAAGLLLLAAQPAMAEVQLVESGGGVVQPGRSLRLSCAASGFAFSSYGMHWVRQAPGKGL
EWVAAISYDGSNKYYADSVKGRFTISRDNSKNTLYLQMNSLRAEDTAVYYCARQADTKYFWGQGTLVTV
SSGGGGSGGGGSGGSALSSELTQDPAVSVALGQTVRITCQGDSLRSYYASWYQQKPGQAPVLVIYGKN
NRPSGIPDRFSGSSSGNTASLTITGAQAEDEADYYCNSRDSSGNVFGGGTKLTVLGAAAVDGGGSGGG
SGGGETPVTPQPPDILLGPLFNDVQNAKLFPDQKTFADAVPNSDPLMILADYRMQQNQSGFDLRHFVNV
NFTLPKEGEKYVPPEGQSLREHIDGLWPVLTRSTENTEKWDSLLPLPEPYVVPGGRFREVYYWDSYFTM
LGLAESGHWDKVADMVANFAHEIDTYGHIPNGNRSYYLSRSQPPFFALMVELLAQHEGDAALKQYLPQM
QKEYAYWMDGVENLQAGQQEKRVVKLQDGTLLNRYWDDRDTPRPESWVEDIATAKSNPNRPATEIYRD
LRSAAASGWDFSSRWMDNPQQLNTLRTTSIVPVDLNSLMFKMEKILARASKAAGDNAMANQYETLANAR
QKGIEKYLWNDQQGWYADYDLKSHKVRNQLTAAALFPLYVNAAAKDRANKMATATKTHLLQPGGLNTTS
VKSGQQWDAPNGWAPLQWVATEGLQNYGQKEVAMDISWHFLTNVQHTYDREKKLVEKYDVSTTGTGG
GGGEYPLQDGFGWTNGVTLKMLDLICPKEQPCDNVPATRPTVKSATTQPSTKEAQPTPHHHHHH
SEQ ID NO: 49: scFvLm-TreAN-HIS: N-terminally HIS-tagged N-terminal
fragment of TreA C-terminally fused with Listeria monocytogenes ActA
protein specific single chain fragment variable
MGKYLLPTAAAGLLLLAAQPAMAEVQLVESGGGVVQPGRSLRLSCAASGFAFSSYGMHWVRQAPGKGL
EWVAAISYDGSNKYYADSVKGRFTISRDNSKNTLYLQMNSLRAEDTAVYYCARQADTKYFWGQGTLVTV
SSGGGGSGGGGSGGSALSSELTQDPAVSVALGQTVRITCQGDSLRSYYASWYQQKPGQAPVLVIYGKN
NRPSGIPDRFSGSSSGNTASLTITGAQAEDEADYYCNSRDSSGNVFGGGTKLTVLGAAAVDEETPVTPQ
PPDILLGPLFNDVQNAKLFPDQKTFADAVPNSDPLMILADYRMQQNQSGFDLRHFVNVNFTLPKEGEKYV
PHHHHHH
SEQ ID NO: 50: scFvLm-TreAC-HIS: N-terminally HIS-tagged C-terminal
fragment of TreA C-terminally fused with Listeria monocytogenes ActA
protein specific single chain fragment variable
MGKYLLPTAAAGLLLLAAQPAMAEVQLVESGGGVVQPGRSLRLSCAASGFAFSSYGMHWVRQAPGKGL
EWVAAISYDGSNKYYADSVKGRFTISRDNSKNTLYLQMNSLRAEDTAVYYCARQADTKYFWGQGTLVTV
SSGGGGSGGGGSGGSALSSELTQDPAVSVALGQTVRITCQGDSLRSYYASWYQQKPGQAPVLVIYGKN
NRPSGIPDRFSGSSSGNTASLTITGAQAEDEADYYCNSRDSSGNVFGGGTKLTVLGAAAVDNFTLPKEG
EKYVPPEGQSLREHIDGLWPVLTRSTENTEKWDSLLPLPEPYVVPGGRFREVYYWDSYFTMLGLAESGH
WDKVADMVANFAHEIDTYGHIPNGNRSYYLSRSQPPFFALMVELLAQHEGDAALKQYLPQMQKEYAYW
MDGVENLQAGQQEKRVVKLQDGTLLNRYWDDRDTPRPESWVEDIATAKSNPNRPATEIYRDLRSAAAS
GWDFSSRWMDNPQQLNTLRTTSIVPVDLNSLMFKMEKILARASKAAGDNAMANQYETLANARQKGIEKY
LWNDQQGWYADYDLKSHKVRNQLTAAALFPLYVNAAAKDRANKMATATKTHLLQPGGLNTTSVKSGQQ
WDAPNGWAPLQWVATEGLQNYGQKEVAMDISWHFLTNVQHTYDREKKLVEKYDVSTTGTGGGGGEYP
LQDGFGWTNGVTLKMLDLICPKEQPCDNVPATRPTVKSATTQPSTKEAQPTPHHHHHH
SEQ ID NO: 51: HIS-TreAN-ATPase: N-terminally HIS-tagged N-terminal
fragment of TreA C-terminally fused with the ATP synthase epsilon
subunit of Geobacillus
MAHHHHHHEETPVTPQPPDILLGPLFNDVQNAKLFPDQKTFADAVPNSDPLMILADYRMQQNQSGFDLR
HFVNVNFTLPKEGEKYVPGSKTIHVSVVTPDGPVYEDDVEMVSVKAKSGELGILPGHIPLVAPLEISAARL
KKGGKTQYIAVSGGFLEVRPDNVTILAQAAERAEDIDVLRAKARKSGRTPLQSQQDDIDFKRAELALKRA
MNRLSVAEMK
SEQ ID NO: 52: HIS-TreAC-ATPase: N-terminally HIS-tagged C-terminal
fragment of TreA C-terminally fused with the ATP synthase epsilon
subunit of Geobacillus
MAHHHHHHNFTLPKEGEKYVPPEGQSLREHIDGLWPVLTRSTENTEKWDSLLPLPEPYVVPGGRFREVY
YWDSYFTMLGLAESGHWDKVADMVANFAHEIDTYGHIPNGNRSYYLSRSQPPFFALMVELLAQHEGDA
ALKQYLPQMQKEYAYWMDGVENLQAGQQEKRVVKLQDGTLLNRYWDDRDTPRPESWVEDIATAKSNP
NRPATEIYRDLRSAAASGWDFSSRWMDNPQQLNTLRTTSIVPVDLNSLMFKMEKILARASKAAGDNAMA
NQYETLANARQKGIEKYLWNDQQGWYADYDLKSHKVRNQLTAAALFPLYVNAAAKDRANKMATATKTH
LLQPGGLNTTSVKSGQQWDAPNGWAPLQWVATEGLQNYGQKEVAMDISWHFLTNVQHTYDREKKLVE
KYDVSTTGTGGGGGEYPLQDGFGWTNGVTLKMLDLICPKEQPCDNVPATRPTVKSATTQPSTKEAQPT
PGSKTIHVSVVTPDGPVYEDDVEMVSVKAKSGELGILPGHIPLVAPLEISAARLKKGGKTQYIAVSGGFLEV
RPDNVTILAQAAERAEDIDVLRAKARKSGRTPLQSQQDDIDFKRAELALKRAMNRLSVAEMK
SEQ ID NO: 53: HIS-TreAN-KI: N-terminally HIS-tagged N-terminal fragment
of TreA with C-terminal fusion with leucine zipper 1
MAHHHHHHEETPVTPQPPDILLGPLFNDVQNAKLFPDQKTFADAVPNSDPLMILADYRMQQNQSGFDLR
HFVNVNFTLPKEGEKYVPGSVPGSKIAALKEKIAALKE KNAALKWKIAALKE
SEQ ID NO: 54: HIS-TreAC-EI: N-terminally HIS-tagged N-terminal fragment
of TreA with C-terminal fusion leucine zipper 2
MAHHHHHHNFTLPKEGEKYVPPEGQSLREHIDGLWPVLTRSTENTEKWDSLLPLPEPYVVPGGRFREVY
YWDSYFTMLGLAESGHWDKVADMVANFAHEIDTYGHIPNGNRSYYLSRSQPPFFALMVELLAQHEGDA
ALKQYLPQMQKEYAYWMDGVENLQAGQQEKRVVKLQDGTLLNRYWDDRDTPRPESWVEDIATAKSNP
NRPATEIYRDLRSAAASGWDFSSRWMDNPQQLNTLRTTSIVPVDLNSLMFKMEKILARASKAAGDNAMA
NQYETLANARQKGIEKYLWNDQQGWYADYDLKSHKVRNQLTAAALFPLYVNAAAKDRANKMATATKTH
LLQPGGLNTTSVKSGQQWDAPNGWAPLQWVATEGLQNYGQKEVAMDISWHFLTNVQHTYDREKKLVE
KYDVSTTGTGGGGGEYPLQDGFGWTNGVTLKMLDLICPKEQPCDNVPATRPTVKSATTQPSTKEAQPT
PGSEIAALEKEIAALEKENAALEWEIAALEK
SEQ ID NO: 55: HIS-TreAN-EI: N-terminally HIS-tagged N-terminal fragment
of TreA with C-terminal fusion with leucine zipper 1
MAHHHHHHEETPVTPQPPDILLGPLFNDVQNAKLFPDQKTFADAVPNSDPLMILADYRMQQNQSGFDLR
HFVNVNFTLPKEGEKYVPGSVPGSEIAALEKEIAALEKENAALEWEIAALEK
SEQ ID NO: 56: HIS-TreAC-KI: N-terminally HIS-tagged N-terminal fragment
of TreA with C-terminal fusion leucine zipper 2
MAHHHHHHNFTLPKEGEKYVPPEGQSLREHIDGLWPVLTRSTENTEKWDSLLPLPEPYVVPGGRFREVY
YWDSYFTMLGLAESGHWDKVADMVANFAHEIDTYGHIPNGNRSYYLSRSQPPFFALMVELLAQHEGDA
ALKQYLPQMQKEYAYWMDGVENLQAGQQEKRVVKLQDGTLLNRYWDDRDTPRPESWVEDIATAKSNP
NRPATEIYRDLRSAAASGWDFSSRWMDNPQQLNTLRTTSIVPVDLNSLMFKMEKILARASKAAGDNAMA
NQYETLANARQKGIEKYLWNDQQGWYADYDLKSHKVRNQLTAAALFPLYVNAAAKDRANKMATATKTH
LLQPGGLNTTSVKSGQQWDAPNGWAPLQWVATEGLQNYGQKEVAMDISWHFLTNVQHTYDREKKLVE
KYDVSTTGTGGGGGEYPLQDGFGWTNGVTLKMLDLICPKEQPCDNVPATRPTVKSATTQPSTKEAQPT
PGSKIAALKE KIAALKE KNAALKW KIAALKE
SEQ ID NO: 57: rPrP-TreA-HIS: C-terminally HIS-tagged TreA N-terminally
fused with bank vole prion protein
MGKKRPKPGGWNTGGSRYPGQGSPGGNRYPPQGGGTWGQPHGGGWGQPHGGGWGQPHGGGWG
QPHGGGWGQGGGTHNQWNKPSKPKTNMKHVAGAAAAGAVVGGLGGYMLGSAMSRPMIHFGNDWED
RYYRENMNRYPNQVYYRPVDQYNNQNNFVHDCVNITIKQHTVTTTTKGENFTETDVKMMERVVEQMCV
TQYQKESQAYYEGRSVDGGGSGGGSGGGETPVTPQPPDILLGPLFNDVQNAKLFPDQKTFADAVPNSD
PLMILADYRMQQNQSGFDLRHFVNVNFTLPKEGEKYVPPEGQSLREHIDGLWPVLTRSTENTEKWDSLL
PLPEPYVVPGGRFREVYYWDSYFTMLGLAESGHWDKVADMVANFAHEIDTYGHIPNGNRSYYLSRSQP
PFFALMVELLAQHEGDAALKQYLPQMQKEYAYWMDGVENLQAGQQEKRVVKLQDGTLLNRYWDDRDT
PRPESWVEDIATAKSNPNRPATEIYRDLRSAAASGWDFSSRWMDNPQQLNTLRTTSIVPVDLNSLMFKM
EKILARASKAAGDNAMANQYETLANARQKGIEKYLWNDQQGWYADYDLKSHKVRNQLTAAALFPLYVNA
AAKDRANKMATATKTHLLQPGGLNTTSVKSGQQWDAPNGWAPLQWVATEGLQNYGQKEVAMDISWHF
LTNVQHTYDREKKLVEKYDVSTTGTGGGGGEYPLQDGFGWTNGVTLKMLDLICPKEQPCDNVPATRPT
VKSATTQPSTKEAQPTPHHHHHH
SEQ ID NO: 58: rPrP-TreAN-HIS: C-terminally HIS-tagged N-terminal
fragment of TreA N-terminally fused with bank vole prion protein
MGKKRPKPGGWNTGGSRYPGQGSPGGNRYPPQGGGTWGQPHGGGWGQPHGGGWGQPHGGGWG
QPHGGGWGQGGGTHNQWNKPSKPKTNMKHVAGAAAAGAVVGGLGGYMLGSAMSRPMIHFGNDWED
RYYRENMNRYPNQVYYRPVDQYNNQNNFVHDCVNITIKQHTVTTTTKGENFTETDVKMMERVVEQMCV
TQYQKESQAYYEGRSVDGGGSGGGSGGGEETPVTPQPPDILLGPLFNDVQNAKLFPDQKTFADAVPNS
DPLMILADYRMQQNQSGFDLRHFVNVNFTLPKEGEKYVPHHHHHH
SEQ ID NO: 59: rPrP-TreAC-HIS: C-terminally HIS-tagged C-terminal
fragment of TreA N-terminally fused with bank vole prion protein
MGKKRPKPGGWNTGGSRYPGQGSPGGNRYPPQGGGTWGQPHGGGWGQPHGGGWGQPHGGGWG
QPHGGGWGQGGGTHNQWNKPSKPKTNMKHVAGAAAAGAVVGGLGGYMLGSAMSRPMIHFGNDWED
RYYRENMNRYPNQVYYRPVDQYNNQNNFVHDCVNITIKQHTVTTTTKGENFTETDVKMMERVVEQMCV
TQYQKESQAYYEGRSVDNFTLPKEGEKYVPPEGQSLREHIDGLWPVLTRSTENTEKWDSLLPLPEPYVV
PGGRFREVYYWDSYFTMLGLAESGHWDKVADMVANFAHEIDTYGHIPNGNRSYYLSRSQPPFFALMVE
LLAQHEGDAALKQYLPQMQKEYAYWMDGVENLQAGQQEKRVVKLQDGTLLNRYWDDRDTPRPESWV
EDIATAKSNPNRPATEIYRDLRSAAASGWDFSSRWMDNPQQLNTLRTTSIVPVDLNSLMFKMEKILARAS
KAAGDNAMANQYETLANARQKGIEKYLWNDQQGWYADYDLKSHKVRNQLTAAALFPLYVNAAAKDRAN
KMATATKTHLLQPGGLNTTSVKSGQQWDAPNGWAPLQWVATEGLQNYGQKEVAMDISWHFLTNVQHT
YDREKKLVEKYDVSTTGTGGGGGEYPLQDGFGWTNGVTLKMLDLICPKEQPCDNVPATRPTVKSATTQ
PSTKEAQPTPHHHHHH
SEQ ID NO: 60: HIS-TreA^{CWE}: N-terminally HIS-tagged TreA with CWE scar
in linker region
MAHHHHHHEETPVTPQPPDILLGPLFNDVQNAKLFPDQKTFADAVPNSDPLMILADYRMQQNQSGFDLR
HFVNVNFTLPKECWEKYVPPEGQSLREHIDGLWPVLTRSTENTEKWDSLLPLPEPYVVPGGRFREVYYW
DSYFTMLGLAESGHWDKVADMVANFAHEIDTYGHIPNGNRSYYLSRSQPPFFALMVELLAQHEGDAALK
QYLPQMQKEYAYWMDGVENLQAGQQEKRVVKLQDGTLLNRYWDDRDTPRPESWVEDIATAKSNPNRP
ATEIYRDLRSAAASGWDFSSRWMDNPQQLNTLRTTSIVPVDLNSLMFKMEKILARASKAAGDNAMANQY
ETLANARQKGIEKYLWNDQQGWYADYDLKSHKVRNQLTAAALFPLYVNAAAKDRANKMATATKTHLLQP
GGLNTTSVKSGQQWDAPNGWAPLQWVATEGLQNYGQKEVAMDISWHFLTNVQHTYDREKKLVEKYDV
STTGTGGGGGEYPLQDGFGWTNGVTLKMLDLICPKEQPCDNVPATRPTVKSATTQPSTKEAQPTP
SEQ ID NO: 61: TreAN-IntN: N-terminally HIS-tagged N-terminal fragment
of TreA N-terminally fused N-terminal fragment of the split intein DnaE
of Nostoc punctiforme
MAHHHHHHEETPVTPQPPDILLGPLFNDVQNAKLFPDQKTFADAVPNSDPLMILADYRMQQNQSGFDLR
HFVNVNFTLPKECLSYETEILTVEYGLLPIGKIVEKRIECTVYSVDNNGNIYTQPVAQWHDRGEQEVFEYCL
EDGSLIRATKDHKFMTVDGQMLPIDEIFERELDLMRVDNLPNGSGGKL
SEQ ID NO: 62: IntC-TreAC: C-terminally HIS-tagged C-terminal fragment
of TreA N-terminally fused C-terminal fragment of the split intein DnaE
of Nostoc punctiforme
MAASGGTSIKIATRKYLGKQNVYDIGVERDHNFALKNGFIASNCWEKYVPPEGQSLREHIDGLWPVLTRS
TENTEKWDSLLPLPEPYVVPGGRFREVYYWDSYFTMLGLAESGHWDKVADMVANFAHEIDTYGHIPNG
NRSYYLSRSQPPFFALMVELLAQHEGDAALKQYLPQMQKEYAYWMDGVENLQAGQQEKRVVKLQDGT
LLNRYWDDRDTPRPESWVEDIATAKSNPNRPATEIYRDLRSAAASGWDFSSRWMDNPQQLNTLRTTSIV
PVDLNSLMFKMEKILARASKAAGDNAMANQYETLANARQKGIEKYLWNDQQGWYADYDLKSHKVRNQL
TAAALFPLYVNAAAKDRANKMATATKTHLLQPGGLNTTSVKSGQQWDAPNGWAPLQWVATEGLQNYG
QKEVAMDISWHFLTNVQHTYDREKKLVEKYDVSTTGTGGGGGEYPLQDGFGWTNGVTLKMLDLICPKE
QPCDNVPATRPTVKSATTQPSTKEAQPTPHHHHHH
SEQ ID NO: 63: ER-TreAN-HIS: C-terminally HIS-tagged N-terminal fragment
of TreA N-terminally fused with human estradiol receptor fragment
MGKRSKKNSLALSLTADQMVSALLDAEPPILYSEYDPTRPFSEASMMGLLTNLADRELVHMINWAKRVPG
FVDLTLHDQVHLLESAWLEILMIGLVWRSMEHPGKLLFAPNLLLDRNQGKSVEGMVEIFDMLLATSSRFR
MMNLQGEEFVCLKSIILLNSGVYTFLSSTLKSLEEKDHIHRVLDKITDTLIHLMAKAGLTLQQQHQRLAQLLL
ILSHIRHMSNKGMEHLYSMKSKNGGSGVDEETPVTPQPPDILLGPLFNDVQNAKLFPDQKTFADAVPNSD
PLMILADYRMQQNQSGFDLRHFVNVNFTLPKEGEKYVPHHHHHH
SEQ ID NO: 64: ER-TreAC-HIS: C-terminally HIS-tagged C-terminal fragment
of TreA N-terminally fused with human estradiol receptor fragment
MGKRSKKNSLALSLTADQMVSALLDAEPPILYSEYDPTRPFSEASMMGLLTNLADRELVHMINWAKRVPG
FVDLTLHDQVHLLESAWLEILMIGLVWRSMEHPGKLLFAPNLLLDRNQGKSVEGMVEIFDMLLATSSRFR
MMNLQGEEFVCLKSIILLNSGVYTFLSSTLKSLEEKDHIHRVLDKITDTLIHLMAKAGLTLQQQHQRLAQLLL
ILSHIRHMSNKGMEHLYSMKSKNGGSGVDNFTLPKEGEKYVPPEGQSLREHIDGLWPVLTRSTENTEKW
DSLLPLPEPYVVPGGRFREVYYWDSYFTMLGLAESGHWDKVADMVANFAHEIDTYGHIPNGNRSYYLSR
SQPPFFALMVELLAQHEGDAALKQYLPQMQKEYAYWMDGVENLQAGQQEKRVVKLQDGTLLNRYWDD
RDTPRPESWVEDIATAKSNPNRPATEIYRDLRSAAASGWDFSSRWMDNPQQLNTLRTTSIVPVDLNSLM
FKMEKILARASKAAGDNAMANQYETLANARQKGIEKYLWNDQQGWYADYDLKSHKVRNQLTAAALFPLY
VNAAAKDRANKMATATKTHLLQPGGLNTTSVKSGQQWDAPNGWAPLQWVATEGLQNYGQKEVAMDIS
WHFLTNVQHTYDREKKLVEKYDVSTTGTGGGGGEYPLQDGFGWTNGVTLKMLDLICPKEQPCDNVPAT
RPTVKSATTQPSTKEAQPTPHHHHHH
SEQ ID NO: 65: IFNGRA-TreAN-HIS: C-terminally HIS-tagged N-terminal fragment
of TreA N-terminally fused with bovine interferon-gamma receptor A fragment
MASAIPGLSSVPPPTNVTIQAYNLNTVIFWDYPVILQSPMFTVQVMNYEDGKWIDACNTSDHSCNIFSVIN
DPSSSVWGRVKVRVGQEESVYAQSKEFILCKEGKVGPPKLGIRKKENQIIVDIFHPLITVNGKEPEAMYDD
ENTCYTFTYSVFVSINRSETTDKMYTKEEDCNETQCFLNIPVSSLNSQYCVSAEGVSELWAVTTEKSDEL
CITFSVDEETPVTPQPPDILLGPLFNDVQNAKLFPDQKTFADAVPNSDPLMILADYRMQQNQSGFDLRHF
VNVNFTLPKEGEKYVPHHHHHH
SEQ ID NO: 66: IFNGRA-TreAC-HIS: C-terminally HIS-tagged C-terminal fragment
of TreA N-terminally fused with bovine interferon-gamma receptor A fragment
MASAIPGLSSVPPPTNVTIQAYNLNTVIFWDYPVILQSPMFTVQVMNYEDGKWIDACNTSDHSCNIFSVIN
DPSSSVWGRVKVRVGQEESVYAQSKEFILCKEGKVGPPKLGIRKKENQIIVDIFHPLITVNGKEPEAMYDD
ENTCYTFTYSVFVSINRSETTDKMYTKEEDCNETQCFLNIPVSSLNSQYCVSAEGVSELWAVTTEKSDEL
CITFSVDNFTLPKEGEKYVPPEGQSLREHIDGLWPVLTRSTENTEKWDSLLPLPEPYVVPGGRFREVYYW
DSYFTMLGLAESGHWDKVADMVANFAHEIDTYGHIPNGNRSYYLSRSQPPFFALMVELLAQHEGDAALK
QYLPQMQKEYAYWMDGVENLQAGQQEKRVVKLQDGTLLNRYWDDRDTPRPESWVEDIATAKSNPNRP
ATEIYRDLRSAAASGWDFSSRWMDNPQQLNTLRTTSIVPVDLNSLMFKMEKILARASKAAGDNAMANQY
ETLANARQKGIEKYLWNDQQGWYADYDLKSHKVRNQLTAAALFPLYVNAAAKDRANKMATATKTHLLQP
GGLNTTSVKSGQQWDAPNGWAPLQWVATEGLQNYGQKEVAMDISWHFLTNVQHTYDREKKLVEKYDV
STTGTGGGGGEYPLQDGFGWTNGVTLKMLDLICPKEQPCDNVPATRPTVKSATTQPSTKEAQPTPHHH
HHH
SEQ ID NO: 67: Ab-TreA-HIS: C-terminally HIS-tagged TreA with N-terminal
fusion with Amyloid beta p42 peptide
MDAEFRHDSGYEVHHQKLVFFAEDVGSNKGAIIGLMVGGVVIAVDGGGSGGGSGGGETPVTPQPPDILL
GPLFNDVQNAKLFPDQKTFADAVPNSDPLMILADYRMQQNQSGFDLRHFVNVNFTLPKEGEKYVPPEGQ
SLREHIDGLWPVLTRSTENTEKWDSLLPLPEPYVVPGGRFREVYYWDSYFTMLGLAESGHWDKVADMV
ANFAHEIDTYGHIPNGNRSYYLSRSQPPFFALMVELLAQHEGDAALKQYLPQMQKEYAYWMDGVENLQA
GQQEKRVVKLQDGTLLNRYWDDRDTPRPESWVEDIATAKSNPNRPATEIYRDLRSAAASGWDFSSRWM
DNPQQLNTLRTTSIVPVDLNSLMFKMEKILARASKAAGDNAMANQYETLANARQKGIEKYLWNDQQGWY
ADYDLKSHKVRNQLTAAALFPLYVNAAAKDRANKMATATKTHLLQPGGLNTTSVKSGQQWDAPNGWAP
LQWVATEGLQNYGQKEVAMDISWHFLTNVQHTYDREKKLVEKYDVSTTGTGGGGGEYPLQDGFGWTN
GVTLKMLDLICPKEQPCDNVPATRPTVKSATTQPSTKEAQPTPHHHHHH
SEQ ID NO: 68: Ab-TreAN-HIS: C-terminally HIS-tagged N-terminal fragment
of TreA with N-terminal fusion with Amyloid beta p42 peptide
MDAEFRHDSGYEVHHQKLVFFAEDVGSNKGAIIGLMVGGVVIAVDEETPVTPQPPDILLGPLFNDVQNAK
LFPDQKTFADAVPNSDPLMILADYRMQQNQSGFDLRHFVNVNFTLPKEGEKYVPHHHHHH
SEQ ID NO: 69: Ab-TreAC-HIS: C-terminally HIS-tagged C-terminal fragment
of TreA with N-terminal fusion with Amyloid beta p42 peptide
MDAEFRHDSGYEVHHQKLVFFAEDVGSNKGAIIGLMVGGVVIAVDNFTLPKEGEKYVPPEGQSLREHIDG
LWPVLTRSTENTEKWDSLLPLPEPYVVPGGRFREVYYWDSYFTMLGLAESGHWDKVADMVANFAHEID
TYGHIPNGNRSYYLSRSQPPFFALMVELLAQHEGDAALKQYLPQMQKEYAYWMDGVENLQAGQQEKRV
VKLQDGTLLNRYWDDRDTPRPESWVEDIATAKSNPNRPATEIYRDLRSAAASGWDFSSRWMDNPQQLN
TLRTTSIVPVDLNSLMFKMEKILARASKAAGDNAMANQYETLANARQKGIEKYLWNDQQGWYADYDLKS
HKVRNQLTAAALFPLYVNAAAKDRANKMATATKTHLLQPGGLNTTSVKSGQQWDAPNGWAPLQWVATE
GLQNYGQKEVAMDISWHFLTNVQHTYDREKKLVEKYDVSTTGTGGGGGEYPLQDGFGWTNGVTLKML
DLICPKEQPCDNVPATRPTVKSATTQPSTKEAQPTPHHHHHH
SEQ ID NO: 70: GDH-HIS-TreA: Glucose dehydrogenase N-terminally fused to
the TreA (w/o signal peptide) with flexible linker and HIS-tag in between
GDH and TreA
MAGKYLLPTAAAGLLLLAAQPAMADVPLTPSQFAKAKSENFDKKVILSNLNKPHALLWGPDNQIWLTERA
TGKILRVNPESGSVKTVFQVPEIVNDADGQNGLLGFAFHPRFKNNPYIYISGTFKNPKSTDKELPNQTIIRR
YTYKKKTDTLEKPVDLLAGLPSSKDHQGGRLVIGPDQKIYYTIGDQGRNQFAGLFLPNQAQHTPTQQELN
GKDYHTYMGKVLRLNLDGSIPKDNPSFNGVVSHIYTLGHRNPQGLAFTPNGKLLQSDHGPNSDDEINLIV
KGGNYGWPNVAGYKDDSGYAYANYSAAANKSIKDLAQNGLKVAAGVPVTKESEWTGKNFVPPLKTLYT
VQDTYNYNDPTCGEVTYICWPSVAPSSAYVYKGGKKAITGWENTLLVPSLKRGVIFRIKFDPTYSTTYDDA
VPMFKSNNRYRDVIASPDGNVLYVLTDTAGNPVQKDDGSPTNTLENPGSLIKFTYKAKGGKGGSGGSSY
AHHHHHHEETPVTPQPPDILLGPLFNDVQNAKLFPDQKTFADAVPNSDPLMILADYRMQQNQSGFDLRH
FVNVNFTLPKEGEKYVPPEGQSLREHIDGLWPVLTRSTENTEKWDSLLPLPEPYVVPGGRFREVYYWDS
YFTMLGLAESGHWDKVADMVANFAHEIDTYGHIPNGNRSYYLSRSQPPFFALMVELLAQHEGDAALKQY
LPQMQKEYAYWMDGVENLQAGQQEKRVVKLQDGTLLNRYWDDRDTPRPESWVEDIATAKSNPNRPAT
EIYRDLRSAAASGWDFSSRWMDNPQQLNTLRTTSIVPVDLNSLMFKMEKILARASKAAGDNAMANQYET
LANARQKGIEKYLWNDQQGWYADYDLKSHKVRNQLTAAALFPLYVNAAAKDRANKMATATKTHLLQPG
GLNTTSVKSGQQWDAPNGWAPLQWVATEGLQNYGQKEVAMDISWHFLTNVQHTYDREKKLVEKYDVS
TTGTGGGGGEYPLQDGFGWTNGVTLKMLDLICPKEQPCDNVPATRPTVKSATTQPSTKEAQPTP
SEQ ID NO: 71: GDH-HIS-TreAN-EI: Glucose dehydrogenase N-terminally, and
leucine zipper peptide EI C-terminally fused to the N-terminal fragment
of TreAN (w/o signal peptide), which with flexible linker and HIS-tag in
between GDH and TreAN
MAGKYLLPTAAAGLLLLAAQPAMADVPLTPSQFAKAKSENFDKKVILSNLNKPHALLWGPDNQIWLTERA
TGKILRVNPESGSVKTVFQVPEIVNDADGQNGLLGFAFHPRFKNNPYIYISGTFKNPKSTDKELPNQTIIRR
YTYKKKTDTLEKPVDLLAGLPSSKDHQGGRLVIGPDQKIYYTIGDQGRNQFAGLFLPNQAQHTPTQQELN
GKDYHTYMGKVLRLNLDGSIPKDNPSFNGVVSHIYTLGHRNPQGLAFTPNGKLLQSDHGPNSDDEINLIV
KGGNYGWPNVAGYKDDSGYAYANYSAAANKSIKDLAQNGLKVAAGVPVTKESEWTGKNFVPPLKTLYT
VQDTYNYNDPTCGEVTYICWPSVAPSSAYVYKGGKKAITGWENTLLVPSLKRGVIFRIKFDPTYSTTYDDA
VPMFKSNNRYRDVIASPDGNVLYVLTDTAGNPVQKDDGSPTNTLENPGSLIKFTYKAKGGKGGSGGSSY
AHHHHHHEETPVTPQPPDILLGPLFNDVQNAKLFPDQKTFADAVPNSDPLMILADYRMQQNQSGFDLRH
FVNVNFTLPKEGEKYVPGSEIAALEKEIAALEKENAALEWEIAALEK
SEQ ID NO: 72: GDH-HIS-TreAN-KI: Glucose dehydrogenase N-terminally, and
leucine zipper peptide KI C-terminally fused to the N-terminal fragment
of TreAN (w/o signal peptide), which with flexible linker and HIS-tag in
between GDH and TreAN
MAGKYLLPTAAAGLLLLAAQPAMADVPLTPSQFAKAKSENFDKKVILSNLNKPHALLWGPDNQIWLTERA
TGKILRVNPESGSVKTVFQVPEIVNDADGQNGLLGFAFHPRFKNNPYIYISGTFKNPKSTDKELPNQTIIRR
YTYKKKTDTLEKPVDLLAGLPSSKDHQGGRLVIGPDQKIYYTIGDQGRNQFAGLFLPNQAQHTPTQQELN
GKDYHTYMGKVLRLNLDGSIPKDNPSFNGVVSHIYTLGHRNPQGLAFTPNGKLLQSDHGPNSDDEINLIV
KGGNYGWPNVAGYKDDSGYAYANYSAAANKSIKDLAQNGLKVAAGVPVTKESEWTGKNFVPPLKTLYT
VQDTYNYNDPTCGEVTYICWPSVAPSSAYVYKGGKKAITGWENTLLVPSLKRGVIFRIKFDPTYSTTYDDA
VPMFKSNNRYRDVIASPDGNVLYVLTDTAGNPVQKDDGSPTNTLENPGSLIKFTYKAKGGKGGSGGSSY
AHHHHHHEETPVTPQPPDILLGPLFNDVQNAKLFPDQKTFADAVPNSDPLMILADYRMQQNQSGFDLRH
FVNVNFTLPKEGEKYVPGSKIAALKEKIAALKEKNAALKWKIAALKE
SEQ ID NO: 73: GDH-HIS-TreAN-pG: Glucose dehydrogenase N-terminally, and
the immunoglobulin binding domain Domain B1 of protein G C-terminally
fused to the N-terminal fragment of TreAN (w/o signal peptide), which
with flexible linker and HIS-tag in between GDH and TreAN
MAGKYLLPTAAAGLLLLAAQPAMADVPLTPSQFAKAKSENFDKKVILSNLNKPHALLWGPDNQIWLTERA
TGKILRVNPESGSVKTVFQVPEIVNDADGQNGLLGFAFHPRFKNNPYIYISGTFKNPKSTDKELPNQTIIRR
YTYKKKTDTLEKPVDLLAGLPSSKDHQGGRLVIGPDQKIYYTIGDQGRNQFAGLFLPNQAQHTPTQQELN
GKDYHTYMGKVLRLNLDGSIPKDNPSFNGVVSHIYTLGHRNPQGLAFTPNGKLLQSDHGPNSDDEINLIV
KGGNYGWPNVAGYKDDSGYAYANYSAAANKSIKDLAQNGLKVAAGVPVTKESEWTGKNFVPPLKTLYT
VQDTYNYNDPTCGEVTYICWPSVAPSSAYVYKGGKKAITGWENTLLVPSLKRGVIFRIKFDPTYSTTYDDA
VPMFKSNNRYRDVIASPDGNVLYVLTDTAGNPVQKDDGSPTNTLENPGSLIKFTYKAKGGKGGSGGSSY
AHHHHHHEETPVTPQPPDILLGPLFNDVQNAKLFPDQKTFADAVPNSDPLMILADYRMQQNQSGFDLRH
FVNVNFTLPKEGEKYVPGSTYKLILNGKTLKGETTTEAVDAATAEKVFKQYANDNGVDGEWYDAATKTF
TVTE
SEQ ID NO: 74: GDH-HIS-TreAN-pA: Glucose dehydrogenase N-terminally, and
the immunoglobulin binding domain Domain B1 of protein G C-terminally
fused to the N-terminal fragment of TreAN (w/o signal peptide), which with
flexible linker and HIS-tag in between GDH and TreAN
MADVPLTPSQFAKAKSENFDKKVILSNLNKPHALLWGPDNQIWLTERATGKILRVNPESGSVKTVFQVPEI
VNDADGQNGLLGFAFHPRFKNNPYIYISGTFKNPKSTDKELPNQTIIRRYTYKKKTDTLEKPVDLLAGLPSS
KDHQGGRLVIGPDQKIYYTIGDQGRNQFAGLFLPNQAQHTPTQQELNGKDYHTYMGKVLRLNLDGSIPK
DNPSFNGVVSHIYTLGHRNPQGLAFTPNGKLLQSDHGPNSDDEINLIVKGGNYGWPNVAGYKDDSGYAY
ANYSAAANKSIKDLAQNGLKVAAGVPVTKESEWTGKNFVPPLKTLYTVQDTYNYNDPTCGEVTYICWPS
VAPSSAYVYKGGKKAITGWENTLLVPSLKRGVIFRIKFDPTYSTTYDDAVPMFKSNNRYRDVIASPDGNVL
YVLTDTAGNPVQKDDGSPTNTLENPGSLIKFTYKAKGGKGGSGGSSYAHHHHHHEETPVTPQPPDILLG
PLFNDVQNAKLFPDQKTFADAVPNSDPLMILADYRMQQNQSGFDLRHFVNVNFTLPKEGEKYVPGSTAD
NKFNKEQQNAFYEILHLPNLNEEQRNGFIQSLKDDPSQSANLLAEAKKLNDAQAPKA
SEQ ID NO: 75: GDH-HIS-TreAN-pL: Glucose dehydrogenase N-terminally, and
the immunoglobulin binding domain B1 of protein G C-terminally fused to
the N-terminal fragment of TreAN (w/o signal peptide), which with flexible
linker and HIS-tag in between GDH and TreAN
MAGKYLLPTAAAGLLLLAAQPAMADVPLTPSQFAKAKSENFDKKVILSNLNKPHALLWGPDNQIWLTERA
TGKILRVNPESGSVKTVFQVPEIVNDADGQNGLLGFAFHPRFKNNPYIYISGTFKNPKSTDKELPNQTIIRR
YTYKKKTDTLEKPVDLLAGLPSSKDHQGGRLVIGPDQKIYYTIGDQGRNQFAGLFLPNQAQHTPTQQELN
GKDYHTYMGKVLRLNLDGSIPKDNPSFNGVVSHIYTLGHRNPQGLAFTPNGKLLQSDHGPNSDDEINLIV
KGGNYGWPNVAGYKDDSGYAYANYSAAANKSIKDLAQNGLKVAAGVPVTKESEWTGKNFVPPLKTLYT
VQDTYNYNDPTCGEVTYICWPSVAPSSAYVYKGGKKAITGWENTLLVPSLKRGVIFRIKFDPTYSTTYDDA
VPMFKSNNRYRDVIASPDGNVLYVLTDTAGNPVQKDDGSPTNTLENPGSLIKFTYKAKGGKGGSGGSSY
AHHHHHHEETPVTPQPPDILLGPLFNDVQNAKLFPDQKTFADAVPNSDPLMILADYRMQQNQSGFDLRH
FVNVNFTLPKEGEKYVPGSAMEEVTIKANLIFANGSTQTAEFKGTFEKATSEAYAYADTLKKDNGEWTVD
VADKGYTLNIKFAG
SEQ ID NO: 76: HIS-TreAC-TreAN-HA: N-terminally HIS-tagged, C-terminally
HA-tagged fusion of the N-terminal and C-terminal fragments of TreA in
alternative frame folded format
MAHHHHHHNFTLPKEGEKYVPPEGQSLREHIDGSYFTMLGLAESGHWDKVADMVANFAHEIDTYGHIPN
GNRSYYLSRSQPPFFALMVELLAQHEGDAALKQYLPQMQKEYAYWMDGVRPESWVEDIATAKSNPNRP
ATEIYRDLRSAAASGWDFSSRWMDNPQQLNTLRTTSIVPVDLNSLMFKMEKILARASKAAGDNAMANQY
ETLANARQKGIEKYLWNDQQGWYADYDLKSHKVRNQLTAAALFPLYVNAAAKDRANKMATATKTHLLQP
GGLNTTSVKSGQQWDAPNGWAPLQWVATEGLQNYGQKEVAMDISWHFLTNVQHTYDREKKLVEKYDV
STTGTGGGGGEYPLQDGFGWTNGVTLKMLDLICPKEQPEFEETPVTPQPPDILLGPLFNDVQNAKLFPD
QKTFADAVPNSDPLMILADYRMQQNQSGFDLRHFVNVNFTLPKEGEKYVPGSYPYDVPDYA
SEQ ID NO: 77: HA-TreAC-TreAN-HIS: N-terminally HA-tagged, C-terminally
HIS-tagged fusion of the N-terminal and C-terminal fragments of TreA in
alternative frame folded format
MAYPYDVPDYAVDNFTLPKEGEKYVPPEGQSLREHIDGLWPVLTRSTENTEKWDSLLPLPEPYVVPGGR
FREVYYWDSYFTMLGLAESGHWDKVADMVANFAHEIDTYGHIPNGNRSYYLSRSQPPFFALMVELLAQH
EGDAALKQYLPQMQKEYAYWMDGVENLQAGQQEKRVVKLQDGTLLNRYWDDRDTPRPESWVEDIATA
KSNPNRPATEIYRDLRSAAASGWDFSSRWMDNPQQLNTLRTTSIVPVDLNSLMFKMEKILARASKAAGD
NAMANQYETLANARQKGIEKYLWNDQQGWYADYDLKSHKVRNQLTAAALFPLYVNAAAKDRANKMATA
TKTHLLQPGGLNTTSVKSGQQWDAPNGWAPLQWVATEGLQNYGQKEVAMDISWHFLTNVQHTYDREK
KLVEKYDVSTTGTGGGGGEYPLQDGFGWTNGVTLKMLDLICPKEQPEFEETPVTPQPPDILLGPLFNDV
QNAKLFPDQKTFADAVPNSDPLMILADYRMQQNQSGFDLRHFVNVNFTLPKEGEKYVPHHHHHH
SEQ ID NO: 78: SA-TreAC-HIS-TreAN-SA: N-terminal and C-terminal fragments
of TreA in alternative frame folded format with N-terminal and C-terminal
fusion with S. aureus binding peptide aptamer and a HIS-tag between both
fragments
MVPHNPGLISLQGVDNFTLPKEGEKYVPPEGQSLREHIDGLWPVLTRSTENTEKWDSLLPLPEPYVVPG
GRFREVYYWDSYFTMLGLAESGHWDKVADMVANFAHEIDTYGHIPNGNRSYYLSRSQPPFFALMVELLA
QHEGDAALKQYLPQMQKEYAYWMDGVENLQAGQQEKRVVKLQDGTLLNRYWDDRDTPRPESWVEDIA
TAKSNPNRPATEIYRDLRSAAASGWDFSSRWMDNPQQLNTLRTTSIVPVDLNSLMFKMEKILARASKAAG
DNAMANQYETLANARQKGIEKYLWNDQQGWYADYDLKSHKVRNQLTAAALFPLYVNAAAKDRANKMAT
ATKTHLLQPGGLNTTSVKSGQQWDAPNGWAPLQWVATEGLQNYGQKEVAMDISWHFLTNVQHTYDRE
KKLVEKYDVSTTGTGGGGGEYPLQDGFGWTNGVTLKMLDLICPHHHHHHEFEETPVTPEETPVTPQPPD
ILLGPLFNDVQNAKLFPDQKTFADAVPNSDPLMILADYRMQQNQSGFDLRHFVNVNFTLPKEGEKYVPGS
VPHNPGLISLQG
SEQ ID NO: 79: GOx-HIS-TreAN-HA: Signal Peptide of Aspergillus oryzae
fused to glucose oxidase of Aspergillus niger with linker containing
HIS-tag to fused to N-terminal fragment of TreA fused to the HA tag
MMVAWWSLFLYGLQVAAPALASNGIEASLLTDPRDVSGRTVDYIIAGGGLTGLTTAARLTENPNISVLVIE
SGSYESDRGPIIEDLNAYGDIFGSSVDHAYETVELATNNQTALIRSGNGLGGSTLVNGGTWTRPHKAQVD
SWETVFGNEGWNWDNVAAYSLQAERARAPNAKQIAAGHYFNASCHGVNGTVHAGPRDTGDDYSPIVK
ALMSAVEDRGVPTKKDFGCGDPHGVSMFPNTLHEDQVRSDAAREWLLPNYQRPNLQVLTGQYVGKVLL
SQNGTTPRAVGVEFGTHKGNTHNVYAKHEVLLAAGSAVSPTILEYSGIGMKSILEPLGIDTVVDLPVGLNL
QDQTTATVRSRITSAGAGQGQAAWFATFNETFGDYSEKAHELLNTKLEQWAEEAVARGGFHNTTALLIQ
YENYRDWIVNHNVAYSELFLDTAGVASFDVWDLLPFTRGYVHILDKDPYLHHFAYDPQYFLNELDLLGQA
AATQLARNISNSGAMQTYFAGETIPGDNLAYDADLSAWTEYIPYHFRPNYHGVGTCSMMPKEMGGVVD
NAARVYGVQGLRVIDGSIPPTQMSSHVMTVFYAMALKISDAILEDYASMQKGKGGSGGSSYAHHHHHHE
ETPVTPQPPDILLGPLFNDVQNAKLFPDQKTFADAVPNSDPLMILADYRMQQNQSGFDLRHFVNVNFTLP
KEGEKYVPGSYPYDVPDYA
SEQ ID NO: 80: HIS-TreA^{N}-CALM: N-terminally HIS-tagged N-terminal
fragment of TreA^{N} with C-terminal fusion of fungal calmodulin
MAHNHHHHEETPVTPQPPDILLGPLFNDVQNAKLFPDQKTFADAVPNSDPLMILADYRMQQNQSGFDLR
HFVNVNFTLPKEGEKYVPGSEEQVSEYKEAFSLFDKDGDGQITTKELGTVMRSLGQNPSESELQDMINE
VDADNNGTIDFPEFLTMMARKMKDTDSEEEIREAFKVFDRDNNGFISAAELRHVMTSIGEKLTDDEVDEMI
READQDGDGRIDYNEFVQLMMQK
SEQ ID NO: 81: M13-likepeptide
RRTLHKAIDTVRAINKLREG
SEQ ID NO: 82: M13-like-TreA^{C}-HIS: M13 like peptide N-terminally fused
to the C-terminally HIS-tagged C-terminal fragment of TreA
MARRTLHKAIDTVRAINKLREGVDNFTLPKEGEKYVPPEGQSLREHIDGLWPVLTRSTENTEKWDSLLPL
PEPYVVPGGRFREVYYWDSYFTMLGLAESGHWDKVADMVANFAHEIDTYGHIPNGNRSYYLSRSQPPF
FALMVELLAQHEGDAALKQYLPQMQKEYAYWMDGVENLQAGQQEKRVVKLQDGTLLNRYWDDRDTPR
PESWVEDIATAKSNPNRPATEIYRDLRSAAASGWDFSSRWMDNPQQLNTLRTTSIVPVDLNSLMFKMEKI
LARASKAAGDNAMANQYETLANARQKGIEKYLWNDQQGWYADYDLKSHKVRNQLTAAALFPLYVNAAA
KDRANKMATATKTHLLQPGGLNTTSVKSGQQWDAPNGWAPLQWVATEGLQNYGQKEVAMDISWHFLT
NVQHTYDREKKLVEKYDVSTTGTGGGGGEYPLQDGFGWTNGVTLKMLDLICPKEQPCDNVPATRPTVK
SATTQPSTKEAQPTPHHHHHH
SEQ ID NO: 83: HIS-TreA^{N}-bCalm: N-terminally HIS-tagged N-terminal
fragment of TreA^{N} with C-terminal fusion of bovine calmodulin
MAHHHHHHEETPVTPQPPDILLGPLFNDVQNAKLFPDQKTFADAVPNSDPLMILADYRMQQNQSGFDLR
HFVNVNFTLPKEGEKYVPGSADQLTEEQIAEFKEAFSLFDKDGDGTITTKELGTVMRSLGQNPTEAELQD
MINEVDADGNGTIDFPEFLTMMARKMKDTDSEEEIREAFRVFDKDGNGYISAAELRHVMTNLGEKLTDEE
VDEMIREADIDGDGQVNYEEFVQMMTAK
SEQ ID NO: 84: CaMKIIp peptide
RRKLKGAILTTMLATRNF
SEQ ID NO: 85: CaMKIIp-TreA^{C}-HIS: CaMKII peptide N-terminally fused
to the C-terminally HIS-tagged C-terminal fragment of TreA
MARRKLKGAILTTMLATRNFVDNFTLPKEGEKYVPPEGQSLREHIDGLWPVLTRSTENTEKWDSLLPLPE
PYVVPGGRFREVYYWDSYFTMLGLAESGHWDKVADMVANFAHEIDTYGHIPNGNRSYYLSRSQPPFFA
LMVELLAQHEGDAALKQYLPQMQKEYAYWMDGVENLQAGQQEKRVVKLQDGTLLNRYWDDRDTPRPE
SWVEDIATAKSNPNRPATEIYRDLRSAAASGWDFSSRWMDNPQQLNTLRTTSIVPVDLNSLMFKMEKILA
RASKAAGDNAMANQYETLANARQKGIEKYLWNDQQGWYADYDLKSHKVRNQLTAAALFPLYVNAAAKD
RANKMATATKTHLLQPGGLNTTSVKSGQQWDAPNGWAPLQWVATEGLQNYGQKEVAMDISWHFLTNV
QHTYDREKKLVEKYDVSTTGTGGGGGEYPLQDGFGWTNGVTLKMLDLICPKEQPCDNVPATRPTVKSA
TTQPSTKEAQPTPHHHHHH

Although the invention has been described in detail with particular reference to these embodiments, other embodiments can achieve the same results. Variations and modifications of the present invention will be obvious to those skilled in the art and it is intended to cover in the appended claims all such modifications and equivalents. The entire disclosures of all references, applications, patents, and publications cited above are hereby incorporated by reference.

What is claimed is:

1. A composition for use to detect a first analyte in a biological sample, said composition comprising:
a first component consisting of a TreA^{N} fragment of a trehalase enzyme split into two fragments, said TreA^{N} fragment consisting of an amino acid sequence set forth in anyone of SEQ ID NO: 80 or SEQ ID NO: 83, said amino acid sequence excluding the HIS-tag, said TreA^{N} fragment fused to a first complexing domain wherein the first complexing domain is a first conserved stable protein sequence selected for fusion with said TreA^{N} fragment; and
a second component consisting of a TreA^{C} fragment of the split trehalase enzyme, said TreA^{C} fragment consisting of an amino acid sequence set forth in SEQ ID NO: 82 or SEQ ID NO: 85, said amino acid sequence excluding the HIS-tag, said TreA^{C} fragment fused to a second complexing domain wherein the second complexing domain is a second conserved stable protein sequence selected for fusion with said TreA^{C};
wherein the first component and second component are mixed with the biological sample and a trehalose substrate;
whereby a presence of the analyte in the biological sample will fuse the first component and the second component to thereby provide a trehalase-catalyzed generation of glucose moieties from the trehalose substrate, said generated glucose moieties detectable and/or measurable by a glucose-detecting technology selected from an electrochemical assay, a colorimetric assay, a fluorometric assay, and a luminescent assay; and
whereby a lack of presence of the analyte in the biological sample will not generate said glucose moieties.

2. The composition according to claim 1, wherein the fusing of the first component and the second component by the first analyte, is inhibited or reduced in the presence of a second analyte in the biological sample.

3. The composition according to claim 1, wherein the first component or the second component additionally comprises a glucose oxidase enzyme or a glucose dehydrogenase enzyme fused to the first complexing domain and/or the second complexing domain.

4. The composition according to claim 1, additionally comprising:
a third component comprising one of a glucose oxidase enzyme preparation and a glucose dehydrogenase enzyme preparation;
wherein the third component is mixed with a mixture of the first component, the second component, and the biological sample whereby if the biological sample comprises one or more analytes capable of complexing the first component and the second component, trehalase-catalyzed generation of glucose moieties from the trehalose substrate will occur, said generated glucose moieties measurable by a glucose-detection assay selected from an electrochemical assay, a colorimetric assay, a fluorometric assay, and a luminescent assay.

5. The composition according to claim 1, additionally comprising:
a third component consisting of a glucose oxidase enzyme preparation or a glucose dehydrogenase enzyme preparation;
a fourth component consisting of peroxidase enzyme preparation; and
a fifth component consisting of a substrate for the peroxidase enzyme preparation,
wherein the third component, fourth component, and fifth component are mixed with a mixture of the first component, the second component, and the biological sample whereby if the biological sample comprises one or more analytes capable of complexing the first component and the second component, trehalase-catalyzed generation of glucose moieties from the trehalose substrate will occur, said generated glucose moieties measurable by a glucose-detection assay selected from an electrochemical assay, a colorimetric assay, a fluorometric assay, and a luminescent assay.

6. A biosensor for detecting a first analyte in a biological sample, the biosensor comprising:
a test strip or a test chip communicable with a test strip reader;
a first component engaged with a surface of the test strip or the test chip, wherein the first component consists of a TreA^{N} fragment of a trehalase enzyme, said TreA^{N} fragment consisting of an amino acid sequence set forth in SEQ ID NO: 80 or SEQ ID NO: 83, said amino acid sequence excluding the HIS-tag, said TreA^{N} fragment fused to a first complexing domain wherein the first complexing domain is a first conserved stable protein sequence selected for fusion with said TreA^{N} fragment; and
a second component engaged with the surface of the test strip or the test chip, wherein the second component consists of a TreA^{C} fragment of the trehalase enzyme, said TreA^{C} fragment consisting of an amino acid sequence set forth in SEQ ID NO: 82 or SEQ ID NO: 85, said amino acid sequence excluding the HIS-tag, said TreA^{C} fragment fused to a second complexing domain wherein the second complexing domain is a second conserved stable protein sequence selected for fusion with said TreA^{C} fragment;
wherein the test strip or the test chip is contactable with the biological sample and with a trehalose substrate whereby a presence of calcium ions in the biological sample will complex the first component and the second component to thereby produce a trehalase-catalyzed generation of glucose moieties from the trehalose substrate, said generated glucose moieties detectable and/or measurable by a glucose-detecting technology selected from one of an electrochemical assay, a colorimetric assay, a fluorometric assay, and a luminescent assay; and
whereby a lack of presence of the in the biological sample will not produce said glucose moieties.

7. The biosensor according to claim 6, wherein the fusing of the first component and the second component by the first analyte, is inhibited or reduced by the presence of a second analyte in the biological sample.

8. A kit for detecting an analyte in a biological sample, said kit comprising:
a biosensor according to claim 6; and
a trehalose substrate preparation,
wherein said trehalose substrate preparation is applicable to said test strip or test chip after a biological sample has been applied to the test strip or the test chip to thereby produce a signal measurable by one of an electrochemical assay, a colorimetric assay, a fluorometric assay, and a luminescent assay if the biological sample comprises the analyte acting as a linking element to fuse the first component and the second component.

9. The kit according to claim 8, additionally comprising a third component consisting of a glucose oxidase enzyme preparation or a glucose dehydrogenase enzyme preparation, said third component applicable to the test strip after application of the biological sample.

10. The kit according to claim 8, additionally comprising:
a third component consisting of a glucose oxidase enzyme preparation or a glucose dehydrogenase enzyme preparation;
a fourth component consisting of a peroxidase enzyme preparation; and
a fifth component consisting of a substrate for the peroxidase enzyme preparation,
wherein the third component, fourth, and fifth component are mixed together and applied to said test strip after the biological sample as has been applied to the test strip.

11. A kit for detecting a presence of lactoferrin in a milk sample, said kit comprising:
a first biosensor according to claim 6 having a first set of components consisting of a first TreA^{N} fragment consisting of the amino acid sequence set forth in SEQ ID NO: 80 and a TreA^{C} fragment consisting of the amino acid sequence set forth in SEQ ID NO: 85;
a second biosensor according to claim 6 having a second set of components consisting of a second TreA^{N} fragment consisting of the amino acid sequence set forth in SEQ ID NO: 83 and the TreA^{C} fragment consisting of the amino acid sequence set forth in SEQ ID NO: 85; and
a trehalose substrate preparation,
wherein said trehalose substrate preparation is applicable to said test strip or test chip after a biological sample has been applied to the test strip or the test chip to thereby produce a signal measurable by one of an electrochemical assay, a colorimetric assay, a fluorometric assay, and a luminescent assay if the biological sample comprises the analyte acting as a linking element to fuse the first component and the second component.

12. The kit according to claim 11, additionally comprising a third component consisting of a glucose oxidase enzyme preparation or a glucose dehydrogenase enzyme preparation, said third component applicable to the test strip after application of the biological sample.

13. The kit according to claim 11, additionally comprising:
a third component consisting of a glucose oxidase enzyme preparation or a glucose dehydrogenase enzyme preparation;
a fourth component consisting of a peroxidase enzyme preparation; and
a fifth component consisting of a substrate for the peroxidase enzyme preparation,
wherein the third component, fourth, and fifth component are mixed together and applied to said test strip after the biological sample has been applied to the test strip.


SEQUENCE LISTING (85 sequences)

SEQ ID NO: 1 - DNA, 20 bp, Artificial Sequence
  Forward primer for the Tn5 (aph) type II (kanamycin resistance) replacement for the entire TrA gene
tatggacagcaagcgaaccg

SEQ ID NO: 2 - DNA, 21 bp, Artificial Sequence
  Reverse primer for the Tn5 (aph) type II (kanamycin resistance) replacement for the entire TreA gene
tcagaagaactcgtcaagaag

SEQ ID NO: 3 - DNA, 53 bp, Artificial Sequence
  Forward primer HIS-TreAFatg-F
tataccatggcacaccatcaccatcaccatgaagaaacaccggtaacaccaca

SEQ ID NO: 4 - DNA, 32 bp, Artificial Sequence
  Reverse primer TreARtaa-R
tatacctaggttaaggtgtgggttgtgcctct

SEQ ID NO: 5 - DNA, 41 bp, Artificial Sequence
  Reverse primer TreA-BamHI-N-R
taattcctaggtcaggatcccggaacatatttctcgccttc

SEQ ID NO: 6 - DNA, 30 bp, Artificial Sequence
  Forward primer TreAFatg-F
tataccatggaagaaacaccggtaacacca

SEQ ID NO: 7 - DNA, 53 bp, Artificial Sequence
  Reverse primer TreA-TruncN-R
taattcctaggtcaatggtgatggtgatggtgcggaacatatttctcgccttc

SEQ ID NO: 8 - DNA, 50 bp, Artificial Sequence
  Forward primer TreA-TruncC-F
taattccatggcacaccatcaccatcaccataatttcaccctgccgaaag

SEQ ID NO: 9 - DNA, 38 bp, Artificial Sequence
  Forward primer TreA-SalI-C-F
taattccatggcagtcgacaatttcaccctgccgaaag

SEQ ID NO: 10 - DNA, 50 bp, Artificial Sequence
  Reverse primer TreA-HIS-R
tatacctaggttaatggtgatggtgatggtgaggtgtgggttgtgcctct

SEQ ID NO: 11 - DNA, 63 bp, Artificial Sequence
  Forward primer HA-TreA-N-F
tataccatggcatacccatacgatgttccagattacgctgtcgacgaagaaacaccggtaaca

SEQ ID NO: 12 - DNA, 64 bp, Artificial Sequence
  Reverse primer TreA-N-HA-R
tatacctaggtcaagcgtaatctggaacatcgtatgggtaggatcccggaacatatttctcgcc

SEQ ID NO: 13 - DNA, 63 bp, Artificial Sequence
  Forward primer HA-TreA-C-F
tataccatggcatacccatacgatgttccagattacgctgtcgacaatttcaccctgccgaaa

SEQ ID NO: 14 - protein, 55 aa, Artificial Sequence
  Protein G
TYKLILNGKTLKGETTTEAVDAATAEKVFKQYANDNGVDGEWTYDAATKTFTVTE

SEQ ID NO: 15 - protein, 60 aa, Artificial Sequence
  Protein A
TADNKFNKEQQNAFYEILHLPNLNEEQRNGFIQSLKDDPSQSANLLAEAKKLNDAQAPKA

SEQ ID NO: 16 - protein, 65 aa, Artificial Sequence
  Protein L
AMEEVTIKANLIFANGSTQTAEFKGTFEKATSEAYAYADTLKKDNGEWTVDVADKGYTLNIKFAG

SEQ ID NO: 17 - protein, 543 aa, Artificial Sequence
  N-terminally HIS-tagged TreA peptide
MAHHHHHHEETPVTPQPPDILLGPLFNDVQNAKLFPDQKTFADAVPNSDPLMILADYRMQQNQSGFDLRHFVNVNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTP

SEQ ID NO: 18 - protein, 542 aa, Artificial Sequence
  C-terminally HIS-tagged TreA peptide
MEETPVTPQPPDILLGPLFNDVQNAKLFPDQKTFADAVPNSDPLMILADYRMQQNQSGFDLRHFVNVNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTPHHHHHH

SEQ ID NO: 19 - protein, 549 aa, Artificial Sequence
  N- and C- terminally HIS-tagged TreA peptide
MAHHHHHHEETPVTPQPPDILLGPLFNDVQNAKLFPDQKTFADAVPNSDPLMILADYRMQQNQSGFDLRHFVNVNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTPHHHHHH

SEQ ID NO: 20 - protein, 89 aa, Artificial Sequence
  N-terminally HIS-tagged N-terminal fragment of TreA peptide
MAHHHHHHEETPVTPQPPDILLGPLFNDVQNAKLFPDQKTFADAVPNSDPLMILADYRMQQNQSGFDLRHFVNVNFTLPKEGEKYVPGS

SEQ ID NO: 21 - protein, 86 aa, Artificial Sequence
  C-terminally HIS-tagged N-terminal fragment of TreA peptide
MEETPVTPQPPDILLGPLFNDVQNAKLFPDQKTFADAVPNSDPLMILADYRMQQNQSGFDLRHFVNVNFTLPKEGEKYVPHHHHHH

SEQ ID NO: 22 - protein, 477 aa, Artificial Sequence
  N-terminally HIS-tagged C-terminal fragment of TreA peptide
MAHHHHHHNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTP

SEQ ID NO: 23 - protein, 478 aa, Artificial Sequence
  C-terminally HIS-tagged C-terminal fragment of TreA peptide
MAVNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTPHHHHHH

SEQ ID NO: 24 - protein, 98 aa, Artificial Sequence
  N-terminally HA-tagged, C-terminally HIS-tagged N-terminal fragment of TreA peptide
MAYPYDVPDYAVDEETPVTPQPPDILLGPLFNDVQNAKLFPDQKTFADAVPNSDPLMILADYRMQQNQSGFDLRHFVNVNFTLPKEGEKYVPHHHHHH

SEQ ID NO: 25 - protein, 98 aa, Artificial Sequence
  N-terminally HIS-tagged, C-terminally HA-tagged N-terminal fragment of TreA peptide
MAHHHHHHEETPVTPQPPDILLGPLFNDVQNAKLFPDQKTFADAVPNSDPLMILADYRMQQNQSGFDLRHFVNVNFTLPKEGEKYVPGSYPYDVPDYA

SEQ ID NO: 26 - protein, 488 aa, Artificial Sequence
  N-terminally HA-tagged, C-terminally HIS-tagged C-terminal fragment of TreA peptide
MAYPYDVPDYAVDNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTPHHHHHH

SEQ ID NO: 27 - protein, 488 aa, Artificial Sequence
  N-terminally HIS-tagged, C-terminally HA-tagged C-terminal fragment of TreA peptide
MAHHHHHHNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTPGSYPYDVPDYA

SEQ ID NO: 28 - protein, 148 aa, Artificial Sequence
  N-terminally HIS-tagged N-terminal fragment of TreA with C-terminal fusion with immunoglobulin binding Domain B1 of protein G of Streptococcus
MAHHHHHHEETPVTPQPPDILLGPLFNDVQNAKLFPDQKTFADAVPNSDPLMILADYRMQQNQSGFDLRHFVNVNFTLPKEGEKYVPGSVPGSTYKLILNGKTLKGETTTEAVDAATAEKVFKQYANDNGVDGEWTYDAATKTFTVTE

SEQ ID NO: 29 - protein, 267 aa, Artificial Sequence
  N-terminally HIS-tagged N-terminal fragment of TreA with C-terminal fusion with immunoglobulin binding Domain B1 of protein G of Streptococcus
MAHHHHHHNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAA

SEQ ID NO: 30 - protein, 320 aa, Artificial Sequence
  HIV capside protein P24 fused N-terminally to the N-terminal fragment of TreA peptide, C-terminal HIS-tag
MEPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNAMTETLLVQNANPDCKTILKALGPAATLEEMMTACQGVGGPGHKARVLVDEETPVTPQPPDILLGPLFNDVQNAKLFPDQKTFADAVPNSDPLMILADYRMQQNQSGFDLRHFVNVNFTLPKEGEKYVPHHHHHH

SEQ ID NO: 31 - protein, 710 aa, Artificial Sequence
  HIV capside protein P24 fused N-terminally to the C-terminal fragment of TreA peptide, with C-terminal HIS-tag
MEPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNAMTETLLVQNANPDCKTILKALGPAATLEEMMTACQGVGGPGHKARVLVDNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTPHHHHHH

SEQ ID NO: 32 - protein, 154 aa, Artificial Sequence
  N-terminally HIS-tagged N-terminus of TreA peptide, C-terminally fused with the immunoglobulin binding domain of protein A
MAHHHHHHEETPVTPQPPDILLGPLFNDVQNAKLFPDQKTFADAVPNSDPLMILADYRMQQNQSGFDLRHFVNVNFTLPKEGEKYVPGSAMEEVTIKANLIFANGSTQTAEFKGTFEKATSEAYAYADTLKKDNGEWTVDVADKGYTLNIKFAG

SEQ ID NO: 33 - protein, 539 aa, Artificial Sequence
  N-terminally HIS-tagged C-terminus of TreA peptide, C-terminally fused with the immunoglobulin binding domain of protein A
MAHHHHHHNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGVEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNGQHTYDREKKLVEKYDVSTTGTGGGGGEYPLQDGFGWTNGVTLKMLDLICPKEQPCDNVPATRPTVKSATTQPSTKEAQPTPGSTADNKFNKEQQNAFYEILHLPNLNEEQRNGFIQSLKDDPSQSANLLAEAKKLNDAQAPKA

SEQ ID NO: 34 - protein, 149 aa, Artificial Sequence
  N-terminally HIS-tagged N-terminus of TreA peptide, C-terminally fused with the immunoglobulin binding domain of protein L
MAHHHHHHEETPVTPQPPDILLGPLFNDVQNAKLFPDQKTFADAVPNSDPLMILADYRMQQNQSGFDLRHFVNVNFTLPKEGEKYVPGSTADNKFNKEQQNAFYEILHLPNLNEEQRNGFIQSLKDDPSQSANLLAEAKKLNDAQAPKA

SEQ ID NO: 35 - protein, 544 aa, Artificial Sequence
  N-terminally HIS-tagged C-terminus of TreA peptide, C-terminally fused with the immunoglobulin binding domain of protein L
MAHHHHHHNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGVEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNGQHTYDREKKLVEKYDVSTTGTGGGGGEYPLQDGFGWTNGVTLKMLDLICPKEQPCDNVPATRPTVKSATTQPSTKEAQPTPGSAMEEVTIKANLIFANGSTQTAEFKGTFEKATSEAYAYADTLKKDNGEWTVDVADKGYTLNIKFAG

SEQ ID NO: 36 - protein, 12 aa, Artificial Sequence
  Staphylococcus aureus binding peptide SA5-1
VPHNPGLISLQG

SEQ ID NO: 37 - protein, 12 aa, Artificial Sequence
  Mycobacterium avium subsp. paratuberculosis binding peptide Mp3
NYVIHDVPRHPA

SEQ ID NO: 38 - protein, 28 aa, Artificial Sequence
  complementary coiled-coil peptide with leucine zipper motif Ei
EIAALEKEIAALEKENAALEWEIAALEK

SEQ ID NO: 39 - protein, 28 aa, Artificial Sequence
  complementary coiled-coil peptide with leucine zipper motif Ki
KIAALKEKIAALKEKNAALKWKIAALKE

SEQ ID NO: 40 - protein, 101 aa, Artificial Sequence
  N-terminally HIS-tagged N-terminal fragment of TreA with C-terminal fusion with S. aureus binding peptide aptamer, SA5-1
MAHHHHHHEETPVTPQPPDILLGPLFNDVQNAKLFPDQKTFADAVPNSDPLMILADYRMQQNQSGFDLRHFVNVNFTLPKEGEKYVPGSVPHNPGLISLQG

SEQ ID NO: 41 - protein, 100 aa, Artificial Sequence
  N-terminally HIS-tagged N-terminal fragment of TreA with C-terminal fusion with S. aureus binding peptide aptamer, SA5-1
MVPHNPGLISLQGVDEETPVTPQPPDILLGPLFNDVQNAKLFPDQKTFADAVPNSDPLMILADYRMQQNQSGFDLRHFVNVNFTLPKEGEKYVPHHHHHH

SEQ ID NO: 42 - protein, 491 aa, Artificial Sequence
  N-terminally HIS-tagged C-terminal fragment of TreA with C-terminal fusion with S. aureus binding peptide aptamer, SA5-1
MAHHHHHHNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTPGSVPHNPGLISLQG

SEQ ID NO: 43 - protein, 490 aa, Artificial Sequence
  C-terminally HIS-tagged C-terminal fragment of TreA with N-terminal fusion with S. aureus binding peptide aptamer, SA5-1
MVPHNPGLISLQGVDNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTPHHHHHH

SEQ ID NO: 44 - protein, 101 aa, Artificial Sequence
  N-terminally HIS-tagged N-terminal fragment of TreA with C-terminal fusion with M. avium subsp. paratuberculosis binding peptide aptamer, Mp3
MAHHHHHHEETPVTPQPPDILLGPLFNDVQNAKLFPDQKTFADAVPNSDPLMILADYRMQQNQSGFDLRHFVNVNFTLPKEGEKYVPGSNYVIHDVPRHPA

SEQ ID NO: 45 - protein, 101 aa, Artificial Sequence
  C-terminally HIS-tagged N-terminal fragment of TreA with N-terminal fusion with M. avium subsp. paratuberculosis binding peptide aptamer, Mp3
MGNYVIHDVPRHPAVDEETPVTPQPPDILLGPLFNDVQNAKLFPDQKTFADAVPNSDPLMILADYRMQQNQSGFDLRHFVNVNFTLPKEGEKYVPHHHHHH

SEQ ID NO: 46 - protein, 491 aa, Artificial Sequence
  N-terminally HIS-tagged C-terminal fragment of TreA with C-terminal fusion with M. avium subsp. paratuberculosis binding peptide aptamer, Mp3
MAHHHHHHNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTPGSNYVIHDVPRHPA

SEQ ID NO: 47 - protein, 491 aa, Artificial Sequence
  C-terminally HIS-tagged C-terminal fragment of TreA with N-terminal fusion with M. avium subsp. paratuberculosis binding peptide aptamer, Mp3
MGNYVIHDVPRHPAVDNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTPHHHHHH

SEQ ID NO: 48 - protein, 817 aa, Artificial Sequence
  N-terminally HIS-tagged TreA C-terminally fused with Listeria monocytogenes ActA protein specific single chain fragment variable
MGKYLLPTAAAGLLLLAAQPAMAEVQLVESGGGVVQPGRSLRLSCAASGFAFSSYGMHWVRQAPGKGLEWVAAISYDGSNKYYADSVKGRFTISRDNSKNTLYLQMNSLRAEDTAVYYCARQADTKYFWGQGTLVTVSSGGGGSGGGGSGGSALSSELTQDPAVSVALGQTVRITCQGDSLRSYYASWYQQKPGQAPVLVIYGKNNRPSGIPDRFSGSSSGNTASLTITGAQAEDEADYYCNSRDSSGNVFGGGTKLTVLGAAAVDGGGSGGGSGGGETPVTPQPPDILLGPLFNDVQNAKLFPDQKTFADAVPNSDPLMILADYRMQQNQSGFDLRHFVNVNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTPHHHHHH

SEQ ID NO: 49 - protein, 351 aa, Artificial Sequence
  terminally HIS-tagged N-terminal fragment of TreA C-terminally fused with Listeria monocytogenes ActA protein specific single chain fragment variable
MGKYLLPTAAAGLLLLAAQPAMAEVQLVESGGGVVQPGRSLRLSCAASGFAFSSYGMHWVRQAPGKGLEWVAAISYDGSNKYYADSVKGRFTISRDNSKNTLYLQMNSLRAEDTAVYYCARQADTKYFWGQGTLVTVSSGGGGSGGGGSGGSALSSELTQDPAVSVALGQTVRITCQGDSLRSYYASWYQQKPGQAPVLVIYGKNNRPSGIPDRFSGSSSGNTASLTITGAQAEDEADYYCNSRDSSGNVFGGGTKLTVLGAAAVDEETPVTPQPPDILLGPLFNDVQNAKLFPDQKTFADAVPNSDPLMILADYRMQQNQSGFDLRHFVNVNFTLPKEGEKYVPHHHHHH

SEQ ID NO: 50 - protein, 741 aa, Artificial Sequence
  N-terminally HIS-tagged C-terminal fragment of TreA C-terminally fused with Listeria monocytogenes ActA protein specific single chain fragment variable
MGKYLLPTAAAGLLLLAAQPAMAEVQLVESGGGVVQPGRSLRLSCAASGFAFSSYGMHWVRQAPGKGLEWVAAISYDGSNKYYADSVKGRFTISRDNSKNTLYLQMNSLRAEDTAVYYCARQADTKYFWGQGTLVTVSSGGGGSGGGGSGGSALSSELTQDPAVSVALGQTVRITCQGDSLRSYYASWYQQKPGQAPVLVIYGKNNRPSGIPDRFSGSSSGNTASLTITGAQAEDEADYYCNSRDSSGNVFGGGTKLTVLGAAAVDNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTPHHHHHH

SEQ ID NO: 51 - protein, 220 aa, Artificial Sequence
  N-terminally HIS-tagged N-terminal fragment of TreA C-terminally fused with the ATP synthase epsilon subunit of Geobacillus
MAHHHHHHEETPVTPQPPDILLGPLFNDVQNAKLFPDQKTFADAVPNSDPLMILADYRMQQNQSGFDLRHFVNVNFTLPKEGEKYVPGSKTIHVSVVTPDGPVYEDDVEMVSVKAKSGELGILPGHIPLVAPLEISAARLKKGGKTQYIAVSGGFLEVRPDNVTILAQAAERAEDIDVLRAKARKSGRTPLQSQQDDIDFKRAELALKRAMNRLSVAEMK

SEQ ID NO: 52 - protein, 610 aa, Artificial Sequence
  N-terminally HIS-tagged C-terminal fragment of TreA C-terminally fused with the ATP synthase epsilon subunit of Geobacillus
MAHHHHHHNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTPGSKTIHVSVVTPDGPVYEDDVEMVSVKAKSGELGILPGHIPLVAPLEISAARLKKGGKTQYIAVSGGFLEVRPDNVTILAQAAERAEDIDVLRAKARKSGRTPLQSQQDDIDFKRAELALKRAMNRLSVAEMK

SEQ ID NO: 53 - protein, 121 aa, Artificial Sequence
  N-terminally HIS-tagged N-terminal fragment of TreA with C-terminal fusion with leucine zipper 1
MAHHHHHHEETPVTPQPPDILLGPLFNDVQNAKLFPDQKTFADAVPNSDPLMILADYRMQQNQSGFDLRHFVNVNFTLPKEGEKYVPGSVPGSKIAALKEKIAALKEKNAALKWKIAALKE

SEQ ID NO: 54 - protein, 507 aa, Artificial Sequence
  N-terminally HIS-tagged N-terminal fragment of TreA with C-terminal fusion leucine zipper 2
MAHHHHHHNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTPGSEIAALEKEIAALEKENAALEWEIAALEK

SEQ ID NO: 55 - protein, 121 aa, Artificial Sequence
  N-terminally HIS-tagged N-terminal fragment of TreA with C-terminal fusion with leucine zipper 1
MAHHHHHHEETPVTPQPPDILLGPLFNDVQNAKLFPDQKTFADAVPNSDPLMILADYRMQQNQSGFDLRHFVNVNFTLPKEGEKYVPGSVPGSEIAALEKEIAALEKENAALEWEIAALEK

SEQ ID NO: 56 - protein, 507 aa, Artificial Sequence
  N-terminally HIS-tagged N-terminal fragment of TreA with C-terminal fusion leucine zipper 2
MAHHHHHHNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTPGSKIAALKEKIAALKEKNAALKWKIAALKE

SEQ ID NO: 57 - protein, 763 aa, Artificial Sequence
  C-terminally HIS-tagged TreA N-terminally fused with bank vole prion protein
MGKKRPKPGGWNTGGSRYPGQGSPGGNRYPPQGGGTWGQPHGGGWGQPHGGGWGQPHGGGWGQPHGGGWGQGGGTHNQWNKPSKPKTNMKHVAGAAAAGAVVGGLGGYMLGSAMSRPMIHFGNDWEDRYYRENMNRYPNQVYYRPVDQYNNQNNFVHDCVNITIKQHTVTTTTKGENFTETDVKMMERVVEQMCVTQYQKESQAYYEGRSVDGGGSGGGSGGGETPVTPQPPDILLGPLFNDVQNAKLFPDQKTFADAVPNSDPLMILADYRMQQNQSGFDLRHFVNVNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTPHHHHHH

SEQ ID NO: 58 - protein, 308 aa, Artificial Sequence
  C-terminally HIS-tagged N-terminal fragment of TreA N-terminally fused with bank vole prion protein
MGKKRPKPGGWNTGGSRYPGQGSPGGNRYPPQGGGTWGQPHGGGWGQPHGGGWGQPHGGGWGQPHGGGWGQGGGTHNQWNKPSKPKTNMKHVAGAAAAGAVVGGLGGYMLGSAMSRPMIHFGNDWEDRYYRENMNRYPNQVYYRPVDQYNNQNNFVHDCVNITIKQHTVTTTTKGENFTETDVKMMERVVEQMCVTQYQKESQAYYEGRSVDGGGSGGGSGGGEETPVTPQPPDILLGPLFNDVQNAKLFPDQKTFADAVPNSDPLMILADYRMQQNQSGFDLRHFVNVNFTLPKEGEKYVPHHHHHH

SEQ ID NO: 59 - protein, 687 aa, Artificial Sequence
  C-terminally HIS-tagged C-terminal fragment of TreA N-terminally fused with bank vole prion protein
MGKKRPKPGGWNTGGSRYPGQGSPGGNRYPPQGGGTWGQPHGGGWGQPHGGGWGQPHGGGWGQPHGGGWGQGGGTHNQWNKPSKPKTNMKHVAGAAAAGAVVGGLGGYMLGSAMSRPMIHFGNDWEDRYYRENMNRYPNQVYYRPVDQYNNQNNFVHDCVNITIKQHTVTTTTKGENFTETDVKMMERVVEQMCVTQYQKESQAYYEGRSVDNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTPHHHHHH

SEQ ID NO: 60 - protein, 544 aa, Artificial Sequence
  N-terminally HIS-tagged TreA with CWE scar in linker region
MAHHHHHHEETPVTPQPPDILLGPLFNDVQNAKLFPDQKTFADAVPNSDPLMILADYRMQQNQSGFDLRHFVNVNFTLPKECWEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTP

SEQ ID NO: 61 - protein, 189 aa, Artificial Sequence
  N-terminally HIS-tagged N-terminal fragment of TreA N-terminally fused N-terminal fragment of the split intein DnaE of Nostoc punctiforme
MAHHHHHHEETPVTPQPPDILLGPLFNDVQNAKLFPDQKTFADAVPNSDPLMILADYRMQQNQSGFDLRHFVNVNFTLPKECLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNGSGGKL

SEQ ID NO: 62 - protein, 512 aa, Artificial Sequence
  C-terminally HIS-tagged C-terminal fragment of TreA N-terminally fused C-terminal fragment of the split intein DnaE of Nostoc punctiforme
MAASGGTSIKIATRKYLGKQNVYDIGVERDHNFALKNGFIASNCWEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTPHHHHHH

SEQ ID NO: 63 - protein, 327 aa, Artificial Sequence
  C-terminally HIS-tagged N-terminal fragment of TreA N-terminally fused with human estradiol receptor fragment
MGKRSKKNSLALSLTADQMVSALLDAEPPILYSEYDPTRPFSEASMMGLLTNLADRELVHMINWAKRVPGFVDLTLHDQVHLLESAWLEILMIGLVWRSMEHPGKLLFAPNLLLDRNQGKSVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKSKNGGSGVDEETPVTPQPPDILLGPLFNDVQNAKLFPDQKTFADAVPNSDPLMILADYRMQQNQSGFDLRHFVNVNFTLPKEGEKYVPHHHHHH

SEQ ID NO: 64 - protein, 717 aa, Artificial Sequence
  C-terminally HIS-tagged C-terminal fragment of TreA N-terminally fused with human estradiol receptor fragment
MGKRSKKNSLALSLTADQMVSALLDAEPPILYSEYDPTRPFSEASMMGLLTNLADRELVHMINWAKRVPGFVDLTLHDQVHLLESAWLEILMIGLVWRSMEHPGKLLFAPNLLLDRNQGKSVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKSKNGGSGVDNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTPHHHHHH

SEQ ID NO: 65 - protein, 304 aa, Artificial Sequence
  C-terminally HIS-tagged N-terminal fragment of TreA N-terminally fused with bovine interferon-gamma receptor A fragment
MASAIPGLSSVPPPTNVTIQAYNLNTVIFWDYPVILQSPMFTVQVMNYEDGKWIDACNTSDHSCNIFSVINDPSSSVWGRVKVRVGQEESVYAQSKEFILCKEGKVGPPKLGIRKKENQIIVDIFHPLITVNGKEPEAMYDDENTCYTFTYSVFVSINRSETTDKMYTKEEDCNETQCFLNIPVSSLNSQYCVSAEGVSELWAVTTEKSDELCITFSVDEETPVTPQPPDILLGPLFNDVQNAKLFPDQKTFADAVPNSDPLMILADYRMQQNQSGFDLRHFVNVNFTLPKEGEKYVPHHHHHH

SEQ ID NO: 66 - protein, 694 aa, Artificial Sequence
  C-terminally HIS-tagged C-terminal fragment of TreA N-terminally fused with bovine interferon-gamma receptor A fragment
MASAIPGLSSVPPPTNVTIQAYNLNTVIFWDYPVILQSPMFTVQVMNYEDGKWIDACNTSDHSCNIFSVINDPSSSVWGRVKVRVGQEESVYAQSKEFILCKEGKVGPPKLGIRKKENQIIVDIFHPLITVNGKEPEAMYDDENTCYTFTYSVFVSINRSETTDKMYTKEEDCNETQCFLNIPVSSLNSQYCVSAEGVSELWAVTTEKSDELCITFSVDNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTPHHHHHH

SEQ ID NO: 67 - protein, 596 aa, Artificial Sequence
  C-terminally HIS-tagged TreA with N-terminal fusion with Amyloid beta p42 peptide
MDAEFRHDSGYEVHHQKLVFFAEDVGSNKGAIIGLMVGGVVIAVDGGGSGGGSGGGETPVTPQPPDILLGPLFNDVQNAKLFPDQKTFADAVPNSDPLMILADYRMQQNQSGFDLRHFVNVNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTPHHHHHH

SEQ ID NO: 68 - protein, 130 aa, Artificial Sequence
  C-terminally HIS-tagged N-terminal fragment of TreA with N-terminal fusion with Amyloid beta p42 peptide
MDAEFRHDSGYEVHHQKLVFFAEDVGSNKGAIIGLMVGGVVIAVDEETPVTPQPPDILLGPLFNDVQNAKLFPDQKTFADAVPNSDPLMILADYRMQQNQSGFDLRHFVNVNFTLPKEGEKYVPHHHHHH

SEQ ID NO: 69 - protein, 520 aa, Artificial Sequence
  C-terminally HIS-tagged C-terminal fragment of TreA with N-terminal fusion with Amyloid beta p42 peptide
MDAEFRHDSGYEVHHQKLVFFAEDVGSNKGAIIGLMVGGVVIAVDNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTPHHHHHH

SEQ ID NO: 70 - protein, 1032 aa, Artificial Sequence
  Glucose dehydrogenase N-terminally fused to the TreA with flexible linker and HIS-tag in between GDH and TreA
MAGKYLLPTAAAGLLLLAAQPAMADVPLTPSQFAKAKSENFDKKVILSNLNKPHALLWGPDNQIWLTERATGKILRVNPESGSVKTVFQVPEIVNDADGQNGLLGFAFHPRFKNNPYIYISGTFKNPKSTDKELPNQTIIRRYTYKKKTDTLEKPVDLLAGLPSSKDHQGGRLVIGPDQKIYYTIGDQGRNQFAGLFLPNQAQHTPTQQELNGKDYHTYMGKVLRLNLDGSIPKDNPSFNGVVSHIYTLGHRNPQGLAFTPNGKLLQSDHGPNSDDEINLIVKGGNYGWPNVAGYKDDSGYAYANYSAAANKSIKDLAQNGLKVAAGVPVTKESEWTGKNFVPPLKTLYTVQDTYNYNDPTCGEVTYICWPSVAPSSAYVYKGGKKAITGWENTLLVPSLKRGVIFRIKFDPTYSTTYDDAVPMFKSNNRYRDVIASPDGNVLYVLTDTAGNPVQKDDGSPTNTLENPGSLIKFTYKAKGGKGGSGGSSYAHHHHHHEETPVTPQPPDILLGPLFNDVQNAKLFPDQKTFADAVPNSDPLMILADYRMQQNQSGFDLRHFVNVNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTP

SEQ ID NO: 71 - protein, 606 aa, Artificial Sequence
  Glucose dehydrogenase N-terminally, and leucine zipper peptide EI C-terminally fused to the N-terminal fragment of TreAN, which with flexible linker and HIS-tag in between GDH and TreAN
MAGKYLLPTAAAGLLLLAAQPAMADVPLTPSQFAKAKSENFDKKVILSNLNKPHALLWGPDNQIWLTERATGKILRVNPESGSVKTVFQVPEIVNDADGQNGLLGFAFHPRFKNNPYIYISGTFKNPKSTDKELPNQTIIRRYTYKKKTDTLEKPVDLLAGLPSSKDHQGGRLVIGPDQKIYYTIGDQGRNQFAGLFLPNQAQHTPTQQELNGKDYHTYMGKVLRLNLDGSIPKDNPSFNGVVSHIYTLGHRNPQGLAFTPNGKLLQSDHGPNSDDEINLIVKGGNYGWPNVAGYKDDSGYAYANYSAAANKSIKDLAQNGLKVAAGVPVTKESEWTGKNFVPPLKTLYTVQDTYNYNDPTCGEVTYICWPSVAPSSAYVYKGGKKAITGWENTLLVPSLKRGVIFRIKFDPTYSTTYDDAVPMFKSNNRYRDVIASPDGNVLYVLTDTAGNPVQKDDGSPTNTLENPGSLIKFTYKAKGGKGGSGGSSYAHHHHHHEETPVTPQPPDILLGPLFNDVQNAKLFPDQKTFADAVPNSDPLMILADYRMQQNQSGFDLRHFVNVNFTLPKEGEKYVPGSEIAALEKEIAALEKENAALEWEIAALEK

SEQ ID NO: 72 - protein, 606 aa, Artificial Sequence
  Glucose dehydrogenase N-terminally, and leucine zipper peptide KI C-terminally fused to the N-terminal fragment of TreAN, which with flexible linker and HIS-tag in between GDH and TreAN
MAGKYLLPTAAAGLLLLAAQPAMADVPLTPSQFAKAKSENFDKKVILSNLNKPHALLWGPDNQIWLTERATGKILRVNPESGSVKTVFQVPEIVNDADGQNGLLGFAFHPRFKNNPYIYISGTFKNPKSTDKELPNQTIIRRYTYKKKTDTLEKPVDLLAGLPSSKDHQGGRLVIGPDQKIYYTIGDQGRNQFAGLFLPNQAQHTPTQQELNGKDYHTYMGKVLRLNLDGSIPKDNPSFNGVVSHIYTLGHRNPQGLAFTPNGKLLQSDHGPNSDDEINLIVKGGNYGWPNVAGYKDDSGYAYANYSAAANKSIKDLAQNGLKVAAGVPVTKESEWTGKNFVPPLKTLYTVQDTYNYNDPTCGEVTYICWPSVAPSSAYVYKGGKKAITGWENTLLVPSLKRGVIFRIKFDPTYSTTYDDAVPMFKSNNRYRDVIASPDGNVLYVLTDTAGNPVQKDDGSPTNTLENPGSLIKFTYKAKGGKGGSGGSSYAHHHHHHEETPVTPQPPDILLGPLFNDVQNAKLFPDQKTFADAVPNSDPLMILADYRMQQNQSGFDLRHFVNVNFTLPKEGEKYVPGSKIAALKEKIAALKEKNAALKWKIAALKE

SEQ ID NO: 73 - protein, 633 aa, Artificial Sequence
  Glucose dehydrogenase N-terminally, and the immunoglobulin binding domain Domain B1 of protein G C-terminally fused to the N-terminal fragment of TreAN, which with flexible linker and HIS-tag in between GDH and TreAN
MAGKYLLPTAAAGLLLLAAQPAMADVPLTPSQFAKAKSENFDKKVILSNLNKPHALLWGPDNQIWLTERATGKILRVNPESGSVKTVFQVPEIVNDADGQNGLLGFAFHPRFKNNPYIYISGTFKNPKSTDKELPNQTIIRRYTYKKKTDTLEKPVDLLAGLPSSKDHQGGRLVIGPDQKIYYTIGDQGRNQFAGLFLPNQAQHTPTQQELNGKDYHTYMGKVLRLNLDGSIPKDNPSFNGVVSHIYTLGHRNPQGLAFTPNGKLLQSDHGPNSDDEINLIVKGGNYGWPNVAGYKDDSGYAYANYSAAANKSIKDLAQNGLKVAAGVPVTKESEWTGKNFVPPLKTLYTVQDTYNYNDPTCGEVTYICWPSVAPSSAYVYKGGKKAITGWENTLLVPSLKRGVIFRIKFDPTYSTTYDDAVPMFKSNNRYRDVIASPDGNVLYVLTDTAGNPVQKDDGSPTNTLENPGSLIKFTYKAKGGKGGSGGSSYAHHHHHHEETPVTPQPPDILLGPLFNDVQNAKLFPDQKTFADAVPNSDPLMILADYRMQQNQSGFDLRHFVNVNFTLPKEGEKYVPGSTYKLILNGKTLKGETTTEAVDAATAEKVFKQYANDNGVDGEWTYDAATKTFTVTE

SEQ ID NO: 74 - protein, 616 aa, Artificial Sequence
  lucose dehydrogenase N-terminally, and the immunoglobulin binding domain Domain B1 of protein G C-terminally fused to the N-terminal fragment of TreAN, which with flexible linker and HIS-tag in between GDH and TreAN
MADVPLTPSQFAKAKSENFDKKVILSNLNKPHALLWGPDNQIWLTERATGKILRVNPESGSVKTVFQVPEIVNDADGQNGLLGFAFHPRFKNNPYIYISGTFKNPKSTDKELPNQTIIRRYTYKKKTDTLEKPVDLLAGLPSSKDHQGGRLVIGPDQKIYYTIGDQGRNQFAGLFLPNQAQHTPTQQELNGKDYHTYMGKVLRLNLDGSIPKDNPSFNGVVSHIYTLGHRNPQGLAFTPNGKLLQSDHGPNSDDEINLIVKGGNYGWPNVAGYKDDSGYAYANYSAAANKSIKDLAQNGLKVAAGVPVTKESEWTGKNFVPPLKTLYTVQDTYNYNDPTCGEVTYICWPSVAPSSAYVYKGGKKAITGWENTLLVPSLKRGVIFRIKFDPTYSTTYDDAVPMFKSNNRYRDVIASPDGNVLYVLTDTAGNPVQKDDGSPTNTLENPGSLIKFTYKAKGGKGGSGGSSYAHHHHHHEETPVTPQPPDILLGPLFNDVQNAKLFPDQKTFADAVPNSDPLMILADYRMQQNQSGFDLRHFVNVNFTLPKEGEKYVPGSTADNKFNKEQQNAFYEILHLPNLNEEQRNGFIQSLKDDPSQSANLLAEAKKLNDAQAPKA

SEQ ID NO: 75 - protein, 643 aa, Artificial Sequence
  Glucose dehydrogenase N-terminally, and the immunoglobulin binding domain B1 of protein G C-terminally fused to the N-terminal fragment of TreAN, which with flexible linker and HIS-tag in between GDH and TreAN
MAGKYLLPTAAAGLLLLAAQPAMADVPLTPSQFAKAKSENFDKKVILSNLNKPHALLWGPDNQIWLTERATGKILRVNPESGSVKTVFQVPEIVNDADGQNGLLGFAFHPRFKNNPYIYISGTFKNPKSTDKELPNQTIIRRYTYKKKTDTLEKPVDLLAGLPSSKDHQGGRLVIGPDQKIYYTIGDQGRNQFAGLFLPNQAQHTPTQQELNGKDYHTYMGKVLRLNLDGSIPKDNPSFNGVVSHIYTLGHRNPQGLAFTPNGKLLQSDHGPNSDDEINLIVKGGNYGWPNVAGYKDDSGYAYANYSAAANKSIKDLAQNGLKVAAGVPVTKESEWTGKNFVPPLKTLYTVQDTYNYNDPTCGEVTYICWPSVAPSSAYVYKGGKKAITGWENTLLVPSLKRGVIFRIKFDPTYSTTYDDAVPMFKSNNRYRDVIASPDGNVLYVLTDTAGNPVQKDDGSPTNTLENPGSLIKFTYKAKGGKGGSGGSSYAHHHHHHEETPVTPQPPDILLGPLFNDVQNAKLFPDQKTFADAVPNSDPLMILADYRMQQNQSGFDLRHFVNVNFTLPKEGEKYVPGSAMEEVTIKANLIFANGSTQTAEFKGTFEKATSEAYAYADTLKKDNGEWTVDVADKGYTLNIKFAG

SEQ ID NO: 76 - protein, 472 aa, Artificial Sequence
  N-terminally HIS-tagged, C-terminally HA-tagged fusion of the N-terminal and C-terminal fragments of TreA in alternative frame folded format
MAHHHHHHNFTLPKEGEKYVPPEGQSLREHIDGSYFTMLGLAESGHWDKVADMVANFAHEIDTYGHIPNGNRSYYLSRSQPPFFALMVELLAQHEGDAALKQYLPQMQKEYAYWMDGVRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPEFEETPVTPQPPDILLGPLFNDVQNAKLFPDQKTFADAVPNSDPLMILADYRMQQNQSGFDLRHFVNVNFTLPKEGEKYVPGSYPYDVPDYA

SEQ ID NO: 77 - protein, 542 aa, Artificial Sequence
  N-terminally HA-tagged, C-terminally HIS-tagged fusion of the N-terminal and C-terminal fragments of TreA in alternative frame folded format
MAYPYDVPDYAVDNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPEFEETPVTPQPPDILLGPLFNDVQNAKLFPDQKTFADAVPNSDPLMILADYRMQQNQSGFDLRHFVNVNFTLPKEGEKYVPHHHHHH

SEQ ID NO: 78 - protein, 561 aa, Artificial Sequence
  N-terminal and C-terminal fragments of TreA in alternative frame folded format with N-terminal and C-terminal fusion with S. aureus binding peptide aptamer and a HIS-tag between both fragments
MVPHNPGLISLQGVDNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPHHHHHHEFEETPVTPEETPVTPQPPDILLGPLFNDVQNAKLFPDQKTFADAVPNSDPLMILADYRMQQNQSGFDLRHFVNVNFTLPKEGEKYVPGSVPHNPGLISLQG

SEQ ID NO: 79 - protein, 712 aa, Artificial Sequence
  Signal Peptide of Aspergillus oryzae fused to glucose oxidase of Aspergillus niger with linker containing HIS-tag to fused to N-terminal fragment of TreA fused to the HA tag
MMVAWWSLFLYGLQVAAPALASNGIEASLLTDPRDVSGRTVDYIIAGGGLTGLTTAARLTENPNISVLVIESGSYESDRGPIIEDLNAYGDIFGSSVDHAYETVELATNNQTALIRSGNGLGGSTLVNGGTWTRPHKAQVDSWETVFGNEGWNWDNVAAYSLQAERARAPNAKQIAAGHYFNASCHGVNGTVHAGPRDTGDDYSPIVKALMSAVEDRGVPTKKDFGCGDPHGVSMFPNTLHEDQVRSDAAREWLLPNYQRPNLQVLTGQYVGKVLLSQNGTTPRAVGVEFGTHKGNTHNVYAKHEVLLAAGSAVSPTILEYSGIGMKSILEPLGIDTVVDLPVGLNLQDQTTATVRSRITSAGAGQGQAAWFATFNETFGDYSEKAHELLNTKLEQWAEEAVARGGFHNTTALLIQYENYRDWIVNHNVAYSELFLDTAGVASFDVWDLLPFTRGYVHILDKDPYLHHFAYDPQYFLNELDLLGQAAATQLARNISNSGAMQTYFAGETIPGDNLAYDADLSAWTEYIPYHFRPNYHGVGTCSMMPKEMGGVVDNAARVYGVQGLRVIDGSIPPTQMSSHVMTVFYAMALKISDAILEDYASMQKGKGGSGGSSYAHHHHHHEETPVTPQPPDILLGPLFNDVQNAKLFPDQKTFADAVPNSDPLMILADYRMQQNQSGFDLRHFVNVNFTLPKEGEKYVPGSYPYDVPDYA

SEQ ID NO: 80 - protein, 232 aa, Artificial Sequence
  HIS-TreAN-CALM N-terminally HIS-tagged N-terminal fragment of TreAN with C-terminal fusion of fungal calmodulin
MAHHHHHHEETPVTPQPPDILLGPLFNDVQNAKLFPDQKTFADAVPNSDPLMILADYRMQQNQSGFDLRHFVNVNFTLPKEGEKYVPGSEEQVSEYKEAFSLFDKDGDGQITTKELGTVMRSLGQNPSESELQDMINEVDADNNGTIDFPEFLTMMARKMKDTDSEEEIREAFKVFDRDNNGFISAAELRHVMTSIGEKLTDDEVDEMIREADQDGDGRIDYNEFVQLMMQK

SEQ ID NO: 81 - protein, 20 aa, Artificial Sequence
  M13-like peptide
RRTLHKAIDTVRAINKLREG

SEQ ID NO: 82 - protein, 499 aa, Artificial Sequence
  M13-like-TreAC-HIS M13 like peptide N-terminally fused to the C-terminally HIS- tagged C-terminal fragment of TreA
MARRTLHKAIDTVRAINKLREGVDNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTPHHHHHH

SEQ ID NO: 83 - protein, 237 aa, Artificial Sequence
  HIS-TreAN-bCalm N-terminally HIS-tagged N-terminal fragment of TreAN with C-terminal fusion of bovine calmodulin
MAHHHHHHEETPVTPQPPDILLGPLFNDVQNAKLFPDQKTFADAVPNSDPLMILADYRMQQNQSGFDLRHFVNVNFTLPKEGEKYVPGSADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAK

SEQ ID NO: 84 - protein, 18 aa, Artificial Sequence
  CaMKIIp peptide
RRKLKGAILTTMLATRNF

SEQ ID NO: 85 - protein, 497 aa, Artificial Sequence
  CaMKIIp-TreAC-HIS CaMKII peptide N-terminally fused to the C-terminally HIS- tagged C-terminal fragment of TreA
MARRKLKGAILTTMLATRNFVDNFTLPKEGEKYVPPEGQSLREHIDGLWPVLTRSTENTEKWDSLLPLPEPYVVPGGRFREVYYWDSYFTMLGLAESGHWDKVADMVANFAHEIDTYGHIPNGNRSYYLSRSQPPFFALMVELLAQHEGDAALKQYLPQMQKEYAYWMDGVENLQAGQQEKRVVKLQDGTLLNRYWDDRDTPRPESWVEDIATAKSNPNRPATEIYRDLRSAAASGWDFSSRWMDNPQQLNTLRTTSIVPVDLNSLMFKMEKILARASKAAGDNAMANQYETLANARQKGIEKYLWNDQQGWYADYDLKSHKVRNQLTAAALFPLYVNAAAKDRANKMATATKTHLLQPGGLNTTSVKSGQQWDAPNGWAPLQWVATEGLQNYGQKEVAMDISWHFLTNVQHTYDREKKLVEKYDVSTTGTGGGGGEYPLQDGFGWTNGVTLKMLDLICPKEQPCDNVPATRPTVKSATTQPSTKEAQPTPHHHHHH